(19) United States
(12) Patent Application Publication
(10) Pub. No.: US 2024/0353409 A1
(43) Pub. Date: Oct. 24, 2024

(54) NOVEL SEROLOGY ASSAY FOR THE DETECTION OF PORCINE VIRUSES

(71) Applicant: Revivicor, Inc., Blacksburg, VA (US)

(72) Inventor: Kasinath Venkata Kuravi, Blacksburg, VA (US)

(73) Assignee: Revivicor, Inc., Blacksburg, VA (US)

(21) Appl. No.: 18/634,524

(22) Filed: Apr. 12, 2024

Related U.S. Application Data
(60) Provisional application No. 63/459,490, filed on Apr. 14, 2023.

(51) Int. Cl. G01N 33/569

(52) U.S. Cl. CPC G01N 33/56994; G01N 2333/03; G01N 2469/10; G01N 2469/20; G01N 2474/10

(57) ABSTRACT

The present disclosure provides novel serology methods for detecting viremic or latent zoonotic viral infections, such as porcine cytomegalovirus (PCMV) or Porcine lymphotropic herpesvirus (PLHV) infections, in a subject at any stage of development. More specifically, the present disclosure provides an automated multiplexed immunoassay for the rapid and simultaneous detection of one or more anti-PCMV antibodies or anti-PLHV antibodies in biological fluids of a subject suspected of being infected with PCMV or PLHV.

### CROSS-REFERENCE TO RELATED APPLICATIONS

This application claims the benefit of priority to U.S. Provisional Patent Application No. 63/459,490, filed Apr. 14, 2023, which is hereby incorporated by reference, in its entirety for any and all purposes.

### SEQUENCE LISTING

The instant application contains a Sequence Listing, which has been submitted electronically in XML format and is hereby incorporated by reference in its entirety. Said XML file was created on Jun. 4, 2024, is named 080618-4508_SL.xml, and is 71,987 bytes in size.

### TECHNICAL FIELD

The present disclosure relates generally to novel serology assays for detecting viremic or latent zoonotic viral infections, such as porcine cytomegalovirus (PCMV) or Porcine lymphotropic herpesvirus (PLHV) infections, in a subject at any stage of development.

### BACKGROUND

Porcine cytomegalovirus (PCMV) is an enveloped DNA virus belonging to the family Herpesviridae and the subfamily Beta herpesviridae. Infection with PCMV has previously been known as “inclusion body rhinitis” based on the histopathological characteristics of the disease. PCMV is endemic in nearly all swine populations worldwide, including North America, with seroprevalence approaching 100% in most areas. However, PCMV is not considered to be a pathogen that poses a production risk in an agricultural setting. Infection with PCMV has also not been documented in humans. Yet concerns about transmission through xenotransplantation exist. For example, PCMV infection has been suggested to impact the outcome of xenotransplantation in non-human primate studies.

Polymerase-chain-reaction (PCR)-based methods have been developed by academic and diagnostic laboratories and are primarily being used to detect PCMV infection in various animals. These methods can only detect PCMV infection when the virus is in an active viremic stage. After the virus goes into latency, PCR-based assays are prone to give false negative results. In addition, PCR-based assays are laborious. To this date, none of the diagnostic laboratories offers PCMV serology assays.

Accordingly, there is a need for PCMV serology assays that can be run at any Veterinary Diagnostic laboratory (VDL) under Good Laboratory Practice (GLP) conditions to reliably detect PCMV infection at any time during infection (e.g., latent versus viremic) and in any subject. The present disclosure addresses this need.

### SUMMARY OF THE INVENTION

One aspect of the present disclosure provides a method for detecting a porcine cytomegalovirus (PCMV) in a biological sample from a subject, the method comprising, consisting of, or consisting essentially of, obtaining a serum sample from the subject, diluting the serum sample with a diluent, contacting the diluted serum sample with a PCMV antigen for a time period sufficient to allow binding between the PCMV antigen and a corresponding antibody in the diluted serum sample, and detecting the presence or absence of an antibody from the diluted serum sample that reacts with the PCMV antigen using western blot analysis (e.g., a size based automated serology western blot like WES™, Jess™, Abby™, Peggy Sue™, Sally Sue™ (Bio-techne.com) or similar).

Another aspect of the present disclosure provides a method for detecting an anti-porcine cytomegalovirus (PCMV) antibody in a biological sample from a subject, the method comprising, consisting of, or consisting essentially of: (a) introducing into one or more capillaries of a microfluidic device one or more engineered PCMV antigens; a biological sample from the subject, one or more anti-immunoglobulin antibodies, and one or more chemiluminescent molecules; (b) separating the one or more engineered PCMV antigens electrophoretically; (c) immobilizing the one or more electrophoretically separated engineered PCMV antigens on a capillary wall; (d) contacting the one or more immobilized engineered PCMV antigens with the biological sample from the subject; (e) incubating the one or more immobilized engineered PCMV antigens with the biological sample for about 10 minutes to about 120 minutes; and (f) detecting the binding of the one or more engineered PCMV antigens to the biological sample by immunodetection and/or chemiluminescent detection. In some embodiments, the presence of an immunodetection signal and/or a chemiluminescent signal indicates the presence of one or more anti-PCMV antibodies in the biological sample.

Another aspect of the present disclosure provides an automated serology method (e.g., size based automated serology western blot) for detecting an anti-porcine cytomegalovirus (PCMV) antibody in a porcine animal, the method comprising, consisting of, or consisting essentially of: (a) contacting one or more engineered PCMV antigens with a biological sample from the porcine animal; (b) incubating the one or more engineered PCMV antigens with the biological sample for about 10 minutes to about 120 minutes; and (c) detecting the binding of the one or more engineered PCMV antigens to the biological sample by immunodetection and/or chemiluminescent detection (e.g., an immunodetection signal and/or a chemiluminescent signal).

In some embodiments of the automated serology method (e.g., size based automated serology western blot) described herein, prior to step (a), the one or more engineered PCMV antigens are electrophoretically separated by weight and immobilized on a solid support. In some embodiments, the solid support is a capillary wall of a microfluidic device. In some embodiments, the method is performed in a closed-loop automated capillary-based immunoassay system.

In some embodiments of any of the methods described herein, the one or more engineered PCMV antigens with the biological sample are incubated for about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 100 minutes, about 105 minutes, about 110 minutes, about 115 minutes, or about 120 minutes.

In some embodiments of any of the methods described herein, the anti-PCMV antibody is an IgM or an IgG antibody. In some embodiments, the one or more engineered PCMV antigens are selected from the group consisting of envelope glycoprotein B (gB), envelope glycoprotein H (gH), envelope glycoprotein L (gL), envelope glycoprotein M (gM), envelope glycoprotein N (gN), major tegument phosphoprotein1 (U54A), major tegument phosphoprotein2 (U54B), and U100p. In some embodiments, the one or more engineered PCMV antigens comprise glycoprotein B (gB).

In some embodiments of any of the methods described herein, the one or more engineered PCMV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 25, 26, 27, 28, 29, 30, 31, or 32.

In some embodiments, the one or more engineered PCMV antigens comprise the amino acid sequence of SEQ ID NO: 2, 3, 8, 9, 25, 26, 27, 28, 29, 30, 31, 32, or a combination thereof. In some embodiments, the one or more engineered PCMV antigens comprise the amino acid sequence of SEQ ID NO: 3 or 32. In some embodiments, the one or more engineered PCMV antigens comprise the amino acid sequence of SEQ ID NO: 2, 3, 8, 9, 25, 26, 27, 28, 29, 30, 31 and 32.

In some embodiments of any of the methods described herein, the one or more engineered PCMV antigens are encoded by a polynucleotide sequence selected from SEQ ID NO: 1, 5, 6, 7, 10, 11, or a combination thereof.

In some embodiments of any of the methods described herein, the one or more engineered PCMV antigens comprise glycoprotein gH and/or glycoprotein gL. In some embodiments, the one or more engineered PCMV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 18 or 22. In some embodiments, the one or more engineered PCMV antigens comprise the amino acid sequence of SEQ ID NO: 18 and/or 22.

In some embodiments, the one or more engineered PCMV antigens are encoded by a polynucleotide sequence selected from SEQ ID NO: 17, 21, or a combination thereof.

In some embodiments of any of the methods described herein, the one or more engineered PCMV antigens comprise U100p (gQ).

In some embodiments of any of the methods described herein, the subject is a mammal selected from a primate, a non-human primate, a human, or a porcine animal.

Another aspect of the present disclosure provides a method for detecting a porcine lymphotropic herpesvirus (PLHV) in a biological sample from a subject, comprising, consisting essentially of, or consisting of, obtaining a serum sample from the subject, diluting the serum sample with a diluent, contacting the diluted serum sample with a PLHV antigen for a time period sufficient to allow binding between the PLHV antigen and a corresponding antibody in the diluted serum sample, detecting the presence or absence of an antibody from the diluted serum sample that reacts with the PLHV antigen using western blot analysis.

Another aspect of the present disclosure provides a method for detecting an anti-porcine lymphotropic herpesvirus antibody in a biological sample from a subject, the method comprising, consisting of, or consisting essentially of: (a) introducing into one or more capillaries of a microfluidic device one or more engineered PLHV antigens; a biological sample from the subject, one or more anti-immunoglobulin antibodies, and one or more chemiluminescent molecules; (b) separating the one or more engineered PLHV antigens electrophoretically; (c) immobilizing the one or more electrophoretically separated engineered PLHV antigens on a capillary wall; (d) contacting the one or more immobilized engineered PLHV antigens with the biological sample from the subject; (e) incubating the one or more immobilized engineered PLHV antigens with the biological sample for about 10 minutes to about 120 minutes; and (f) detecting the binding of the one or more engineered PLHV antigens to the biological sample by immunodetection and/or chemiluminescent detection (e.g., an immunodetection signal and/or a chemiluminescent signal).

In some embodiments, the presence of an immunodetection signal and/or a chemiluminescent signal indicates the presence of one or more anti-PLHV antibodies (e.g., anti-PLHV-1 antibodies) in the biological sample.

Another aspect of the present disclosure provides an automated serology method (e.g., size based automated serology western blot) for detecting an anti-porcine lymphotropic herpesvirus antibody in a porcine animal, the method comprising, consisting of, or consisting essentially of: (a) contacting one or more engineered PLHV antigens with a biological sample from the porcine animal; (b) incubating the one or more engineered PLHV antigens with the biological sample for about 10 minutes to about 120 minutes; and (c) detecting the binding of the one or more engineered PLHV antigens to the biological sample by immunodetection and/or chemiluminescent detection.

In some embodiments of the automated serology method described herein, prior to step (a), the one or more engineered PLHV antigens are electrophoretically separated by weight and immobilized on a solid support. In some embodiments, the solid support is a capillary wall of a microfluidic device.

In some embodiments of the methods described herein, the porcine lymphotropic herpesvirus (PLHV) is selected from PLHV-1, PLHV-2, PLHV-3, or a combination thereof.

In some embodiments, the method is performed in a closed-loop automated capillary-based immunoassay system. In some embodiments, the one or more engineered PLHV antigens with the biological sample are incubated for about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 100 minutes, about 105 minutes, about 110 minutes, about 115 minutes, or about 120 minutes. In some embodiments, the anti-PLHV antibody is an IgM or an IgG antibody.

In some embodiments of the methods described herein, the one or more engineered PLHV antigens are selected from the group consisting of envelope glycoprotein B (gB), envelope glycoprotein H (gH), envelope glycoprotein L (gL), envelope glycoprotein M (gM), envelope glycoprotein N (gN), major tegument phosphoprotein1 (U54A), major tegument phosphoprotein2 (U54B), and U100p.

In some embodiments of the methods described herein, the one or more engineered PLHV antigens comprise glycoprotein B (gB).

In some embodiments of the methods described herein, the one or more engineered PLHV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 34, 35, 36, 37, 38, 39, 40, 41, 42, or a combination thereof.

In some embodiments, the one or more engineered PLHV antigens comprise the amino acid sequence of SEQ ID NO: 34, 35, 36, 37, 38, 39, 40, 41, 42, or a combination thereof.

In some embodiments, the one or more engineered PLHV antigens is one or more (a) PLHV-1 antigens comprising the amino acid sequence of SEQ ID NO: 34, 35, 36, or a combination thereof; (b) PLHV-2 antigens comprising the amino acid sequence of SEQ ID NO: 37, 38, 39, or a combination thereof; and/or (c) PLHV-3 antigens comprising the amino acid sequence of SEQ ID NO: 40, 41, 42, or a combination thereof.

In some embodiments of the methods described herein, the one or more engineered PLHV antigens comprise the amino acid sequence of SEQ ID NO: 34, 35, 36, 37, 38, 39, 40, 41, and 42.

### BRIEF DESCRIPTION OF THE DRAWINGS

FIG. 1 shows a schematic illustrating the steps of a porcine cytomegalovirus (PCMV) and porcine lymphotropic herpesvirus (PLHV) serology western blot assay disclosed herein. The serology western blot assay can comprise about six steps. In step 1, about 1-2 ml of whole blood (e.g., biological sample) is collected from a subject (e.g., a porcine animal); in step 2, serum is separated from whole blood by centrifugation; in step 3, separated serum from the subject is aliquoted into small Eppendorf tubes; in step 4, the sample is prepared for an automated capillary electrophoresis (CE) western (e.g., Simple Western™, ProteinSimple's Jess™, or Wes™) and loaded into a CE western (e.g., Wes™) plate; in step 5, the CE western (e.g., Wes™) plate is loaded into a CE western instrument (e.g., Simple Western™ instrument); and in step 6, the results generated by the Simple Western™ instrument are analyzed using a relevant software (e.g., Compass software) to identify, characterize, and quantify an anti-PCMV antibody or an anti-PLHV antibody in the sample collected from the subject.

FIG. 2 shows western blot results generated by Compass software illustrating the effectiveness of a porcine cytomegalovirus (PCMV) serology western blot assay disclosed herein, and demonstrating the detection of anti-PCMV antibodies in porcine animals that were raised outside a biosecure barrier facility (sample #4, #5, and #6; infected) when compared to their littermates (samples #1, #2, and #3) that were raised inside the barrier facility (non-infected). The tested engineered PCMV antigen had two isoforms: a first isoform with a molecular weight (MW) of about 17 kDa and a second isoform with MW of about 40 kDa. The two isoforms were detected using a rabbit anti-histidine (His) primary antibody and anti-pig IgG HRP conjugate. The positive control contained the rabbit anti-His primary antibody and was used to determine the expected molecular weight of the PCMV antigen. The negative control contained no serum.

FIG. 3 shows western blot results of a porcine cytomegalovirus (PCMV) serology western blot assay disclosed herein demonstrating the detection of anti-PCMV antibodies in porcine animals (samples #4, #5, and #6) that were raised outside a biosecure barrier facility (infected) when compared to their littermates (samples #1, #2, and #3) that were raised inside the biosecure barrier facility (non-infected).

FIG. 4 shows western blot results of a porcine cytomegalovirus (PCMV) serology western blot assay disclosed herein demonstrating the detection of anti-PCMV antibodies in porcine animals (samples #4, #5, and #6) that were raised outside a biosecure barrier facility (infected) when compared to their littermates (samples #1, #2, and #3) that were raised inside the biosecure barrier facility (non-infected).

FIG. 5 shows western blot results generated by Compass software illustrating the effectiveness and sensitivity of a porcine cytomegalovirus (PCMV) serology western blot assay disclosed herein and demonstrating the detection of anti-PCMV antibodies at different stages (e.g., 12-103 days) of infection in a pig that was raised outside a biosecure barrier facility. The positive control contained serum from a pig known to be PCMV positive by PCR analysis from an independent veterinary diagnostic laboratory. The positive control was used to determine the expected molecular weight of the PCMV antigens. The negative control contained no serum. At day 12, the PCMV serology western blot assay showed a weak positive signal. A stronger positive signal was detected at day 83, day 88, and day 103 (see also, Table 1).

FIG. 6 shows western blot results generated by Compass software illustrating the effectiveness and sensitivity of a porcine cytomegalovirus (PCMV) serology western blot assay disclosed herein and demonstrating the detection of anti-PCMV antibodies in latent phase of PCMV infection in two out of twenty-one porcine animals that were raised in a biosecure barrier facility (sample #12 and #19). All twenty-one animals received a negative PCMV PCR result from an independent veterinary diagnostic laboratory. The positive control contained serum from a pig known to be PCMV positive by PCR analysis from independent veterinary diagnostic lab and was used to determine the expected molecular weight of the PCMV antigens. Two negative controls were used: the first contained serum from a gnotobiotic pig and the second contained no serum.

FIG. 7: shows western blot results generated by Compass software illustrating the effectiveness and sensitivity of a porcine lymphotropic virus (PLHV)-1 serology western blot assay disclosed herein and demonstrating the detection of anti-PLHV-1 antibodies in a porcine animal raised outside a biosecure barrier facility (sample #3; infected) or porcine animals raised inside a biosecure barrier facility (sample #1 and #2). Samples #1 and #2 had a negative PLHV PCR result from an independent veterinary diagnostic laboratory, while sample #3 had a positive PLHV PCR result. The tested PLHV-1 antigen had a molecular weight of about 31 kDa. The engineered PLHV-1 antigen was detected using a rabbit anti-histidine (His) primary antibody (anti-6×His (“6×His” disclosed as SEQ ID NO: 43)) and anti-pig IgG HRP conjugate. The positive control contained the rabbit anti-His primary antibody and was used to determine the expected molecular weight of the PLHV-1 antigen. The negative control contained no serum (e.g., buffer only).

FIG. 8 shows western blot results generated by Compass software illustrating the effectiveness and sensitivity of a porcine lymphotropic virus (PLHV)-2 serology western blot assay disclosed herein and demonstrating the detection of anti-PLHV-2 antibodies in a porcine animal raised outside a biosecure barrier facility (sample #3; infected) or porcine animals raised inside a biosecure barrier facility (sample #1 and #2). Samples #1 and #2 had a negative PLHV PCR result from an independent veterinary diagnostic laboratory, while sample #3 had a positive PLHV PCR result. The tested PLHV-2 antigen had a molecular weight of about 41 kDa. The engineered PLHV-2 antigen was detected using a rabbit anti-histidine (His) primary antibody and anti-pig IgG HRP conjugate. The positive control contained the rabbit anti-His primary antibody and was used to determine the expected molecular weight of the PLHV-2 antigen. The negative control contained no serum.

FIG. 9 shows western blot results generated by Compass software illustrating the effectiveness and sensitivity of a porcine lymphotropic virus (PLHV)-3 serology western blot assay disclosed herein and demonstrating the detection of anti-PLHV-3 antibodies in a porcine animal raised outside a biosecure barrier facility (sample #3; infected) or porcine animals raised inside a biosecure barrier facility (sample #1 and #2). Samples #1 and #2 had a negative PLHV PCR result from an independent veterinary diagnostic laboratory, while sample #3 had a positive PLHV PCR result. The tested PLHV-3 antigen had a molecular weight of about 40 kDa. The engineered PLHV-3 antigen was detected using a rabbit anti-histidine (His) primary antibody and anti-pig IgG HRP conjugate. The positive control contained the rabbit anti-His primary antibody and was used to determine the expected molecular weight of the PLHV-3 antigen. The negative control contained no serum.

### DETAILED DESCRIPTION

### I. Overview

### A. A Novel PCMV Serology Assay

In the xenotransplantation of pig organs or tissues, the donor pig must be screened for a detailed panel of potential zoonotic microbes prior to the transplantation of their organs in a non-human primate (NHP) or a human. In particular, one of the viruses, PCMV, is quite challenging since it can cross the placenta in pregnant sows. PCMV can also remain in a latent stage of infection for a long time thereby eluding detection by standard screening methods (e.g., polymerase-chain-reaction (PCR) assays). For example, in the case of a 57-year-old patient with terminal heart disease who received the first genetically modified pig heart, the donor pig was screened at a validated diagnostic laboratory by PCR and found to be negative for PCMV in four consecutive tests. Griffith et al., N. Engl. J. Med. 387 (1): 35-44 (2022). The donor animal was derived from a porcine endogenous retrovirus (PERV)-C-negative line. This animal was tested every 3 months using PCR for pathogens that affect porcine or human health, including PERV-A, PERV-B, PERV-C, porcine cytomegalovirus (PCMV), and porcine lymphotropic herpesvirus (PLHV). However, the pig was ultimately later found to have been latently infected with PCMV. PCMV microbial cell-free DNA was ultimately found in the donor heart twenty days after xenotransplantation. There is a need for PCMV serology assays that can be run at any Veterinary Diagnostic laboratory (VDL) under Good Laboratory Practice (GLP) conditions to reliably detect PCMV infection at any time during infection (e.g., latent versus viremic) and in any subject prior to xenotransplantation.

Disclosed herein are novel serology assays. These assays are sensitive and consistently resulted in the detection of both active and latent PCMV infections.

### B. A Novel PLHV Serology Assay

Porcine lymphotropic herpesvirus (PLHV) is gamma herpesvirus that occurs in pig population. Three different species are recognized: PLHV-1, PLHV-2, and PLHV-3. However, these herpesviruses do not present any threat to pig's health because the pig appears to be their natural host. Some data indicate that early weaning piglets to a high health herd environment (e.g., biosecure barrier) can potentially prevent PLHV infection. While PLHVs are absent in humans, the detection and analysis of PLHVs is an important public health issue because PLHVs are related to the Epstein-Barr virus (EBV), which causes health complications in organ transplants. Experimental preclinical data indicated that pig organs that were infected with PLHV-1 caused post-transplantation lymphoproliferative disorder (PTLD) when they were transplanted into PLHV-negative animals (e.g., minipigs). Although, no data currently exist showing PLHV-1, PLHV-2, and PLHV-3 transmission from pigs to humans (or non-human primates, NHP), monitoring donor pigs for PLHV infection may result in better xenotransplant outcomes. To date, no reliable PLHV serology assay exists. Hence, the present disclosure provides a rapid high-throughput PLHV serology western blot technique using the Simple Western™ Automated Western Blot Systems (Simple Wes™; Bio-techne) to detect PLHV-1, PLHV-2, and PLHV-3 anti-IgG antibodies in a biological sample from a subject.

### C. Novel Serology Assays for Reliable Detection of PCMV or PLHV Antibodies in a Sample.

Immunologic methods like enzyme-linked immunosorbent assays (ELISA) and western blots can serve as reliable alternatives to PCR based methods. Western blots can detect infection in the viremic as well as in latent stage. Yet none of the diagnostic laboratories offers PCMV serology assays. Moreover, all published PCMV serology assays are cumbersome, less sensitive, and unreliable.

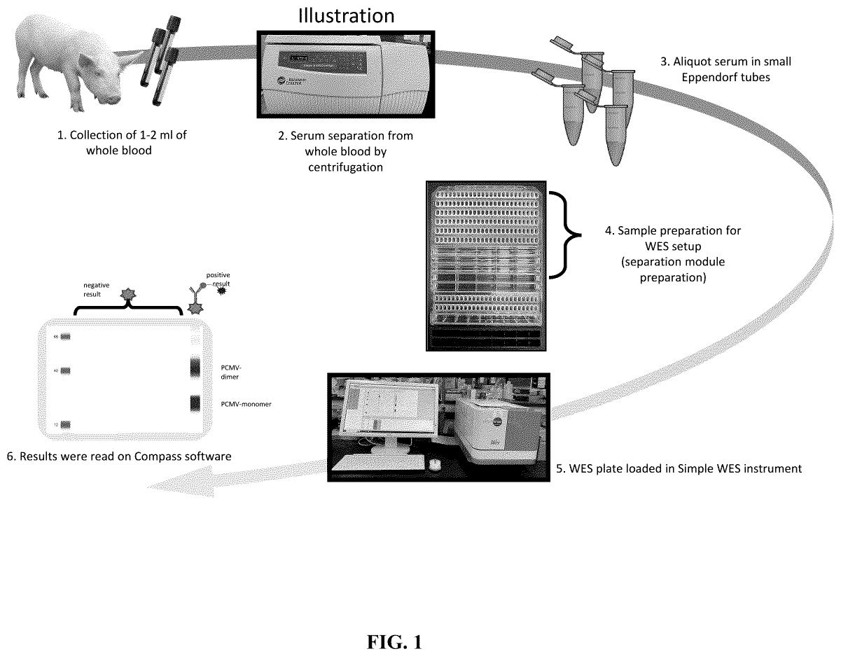

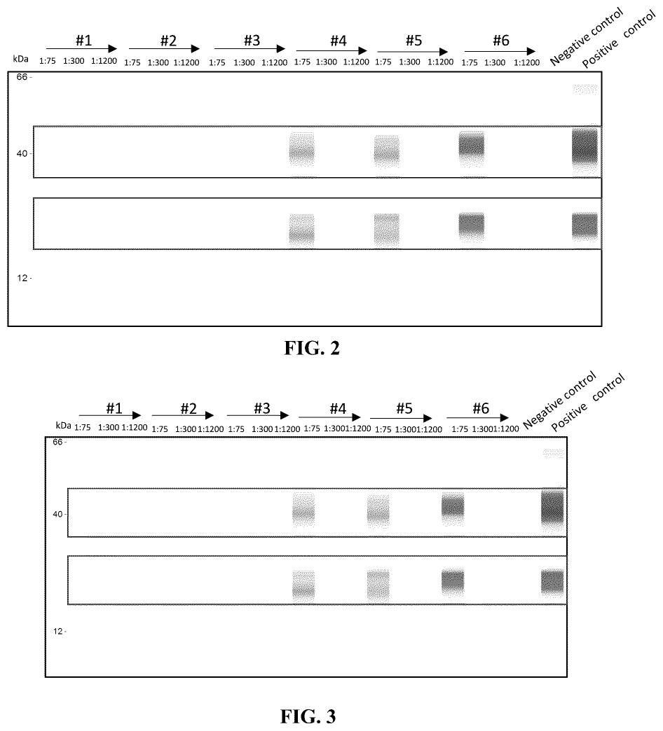

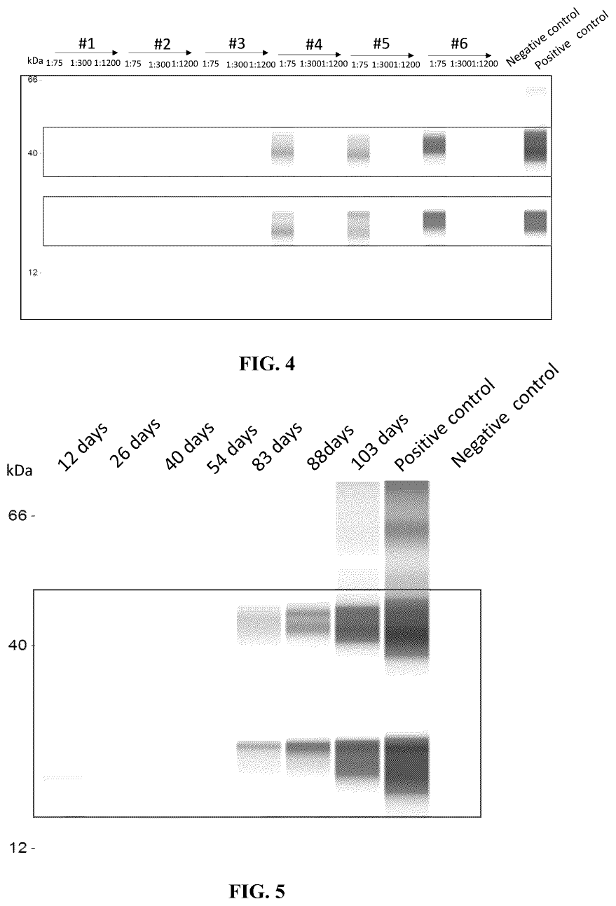

The methods described herein provide a rapid high-throughput PCMV, or PLHV serology western blot technique that uses the Simple Wes™ instrument to detect PCMV or PLHV anti-IgG antibodies in a sample from a subject. FIG. 1 illustrates a process used in the PCMV and PLHV serology western blot assay disclosed herein. FIGS. 2-4 further show western blot results generated using a PCMV serology western blot assay disclosed herein. In particular, FIGS. 2-4 demonstrate the detection of anti-PCMV antibodies in porcine animals (samples #4, #5, and #6) that were raised outside a biosecure barrier facility (e.g., infected) when compared to their littermates (samples #1, #2, and #3) that were raised inside the biosecure barrier facility (e.g., non-infected). As used herein, a “barrier” or a “biosecure barrier” is a Source Animal Facility (SAF) for generating designated pathogen-free (DPF) pigs to serve as donors of viable organs, tissues, or cells for xenotransplantation into clinical subjects.

The sensitivity of the porcine cytomegalovirus (PCMV) serology western blot assay disclosed herein is further shown in FIG. 5 and Table 1. In particular, the PCMV serology western blot assay was as sensitive as a PCR-based assay. In some embodiments, the PCMV serology western blot assay was more sensitive than a PCR-based assay. For example, the PCMV serology western blot assay detected maternal antibodies in 12-day old animals, animals with a latent infection, and animals in early stage viremia. Table 1 shows a comparison between the sensitivity of a well-established PCR-based assay and the novel PCMV serology western blot assay described herein over time.

Table 1 shows the improvement and the surprising sensitivity of the PCMV serology western blot assay for detecting maternal antibodies and antibodies produced during early-stage viremia.

TABLE 1
Timeline of results obtained by independent veterinary
diagnostic lab and PCMV serological western blot assay

| Age (days) | PCR analysis* | PCMV serology assay |
|---|---|---|
| 12 | Negative | weak positive (maternal antibodies) |
| 19 | Negative | — |
| 26 | Suspect | Negative |
| 32 | Negative | — |
| 40 | Negative | Negative |
| 54 | Negative | Negative |
| 71 | Negative | — |
| 83 | POSITIVE | POSITIVE |
| 88 | POSITIVE | POSITIVE |
| 103 | — | POSITIVE |

*Independent Veterinary diagnostic lab

Serum samples were collected in animals between 12 and 103 days of age and tested for PCMV at an independent veterinary diagnostic laboratory via PCR-based assay and/or PCMV serology western blot assay. As shown in Table 1, at day 12, PCR results from the independent veterinary diagnostic laboratory were negative, but the PCMV serology western blot assay showed a weak positive signal (FIG. 5). Thus, the PCMV serology western blot assay can detect passively transmitted maternal antibodies in a biological sample. At day 83 and day 88, PCR results from the independent veterinary diagnostic laboratory and the PCMV serology western blot assay both showed positive signals. See also FIG. 5. At day 103, the PCMV serology western blot assay continued to show positive results. These results demonstrate the PCMV serology western blot assay's ability to detect antibodies during PCMV early stage viremia.

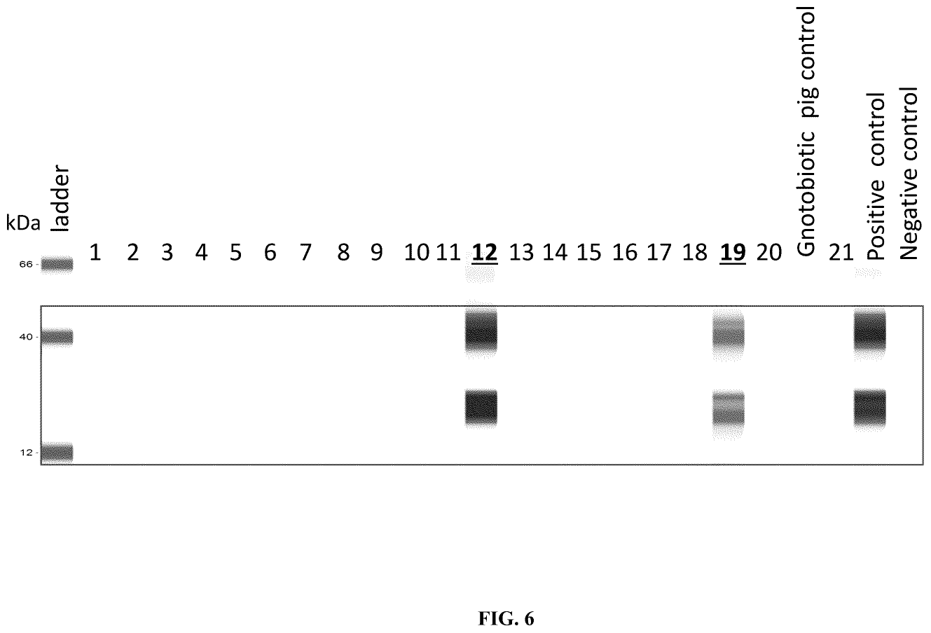

FIG. 6 shows that the PCMV serology western blot assay described herein can also detect anti-PCMV antibodies in a latent phase of PCMV infection. Surprisingly, the PCMV serology western blot assay detected anti-PCMV antibodies in animals that received a negative PCMV PCR result from an independent veterinary diagnostic laboratory. Each of the twenty-one animals tested in FIG. 6 had received a negative PCMV PCR result from an independent veterinary diagnostic laboratory. Yet, FIG. 6 shows that two out of twenty-one porcine animals were positive for anti-PCMV antibodies using the PCMV serology western blot assay described herein. The two animals were in different age groups. Sample #12 was about >3 months of age and sample #19 was about 14 months of age. Thus, the PCMV serology western blot assay described herein robustly detected PCMV infection in latent stage across the lifespan of the porcine animals, despite a prior negative PCR results.

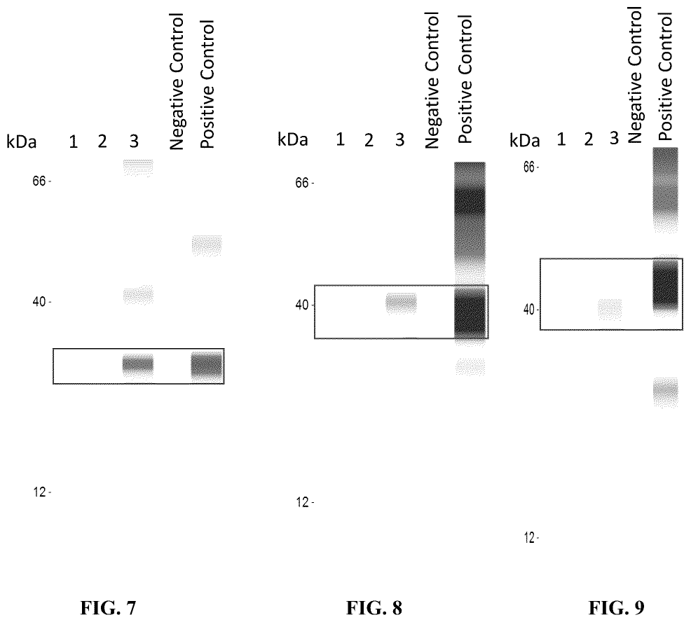

The serology western blot assay described herein also robustly detected PLHV-1, PLHV-2, and PLHV-3 antibodies in samples. FIG. 7 shows the detection of anti-PLHV-1 antibodies in a porcine animal raised outside a biosecure barrier facility (sample #3; infected) but not in porcine animals raised inside a biosecure barrier facility (sample #1 and #2). Samples #1 and #2 had a negative PLHV PCR result from an independent veterinary diagnostic laboratory, while sample #3 had a positive PLHV PCR result. FIG. 8 shows the detection of anti-PLHV-2 antibodies in a porcine animal raised outside a biosecure barrier facility (sample #3; infected) but not in porcine animals raised inside a biosecure barrier facility (sample #1 and #2). Samples #1 and #2 had a negative PLHV PCR result from an independent veterinary diagnostic lab, while sample #3 had a positive PLHV PCR result. FIG. 9 shows the detection of anti-PLHV-3 antibodies in a porcine animal raised outside a biosecure barrier facility (sample #3; infected) but not in porcine animals raised inside a biosecure barrier facility (sample #1 and #2). Samples #1 and #2 had a negative PLHV PCR result from an independent veterinary diagnostic lab, while sample #3 had a positive PLHV PCR result.

The serology western blot assay described herein distinguished between PLHV-1, PLHV-2, and PLHV-3 (See FIGS. 7, 8, and 9). For example, the tested PLHV-1 antigen had a molecular weight of about 31 kDa (FIG. 7). The tested PLHV-2 antigen had a molecular weight of about 41 kDa (FIG. 8). The tested PLHV-3 antigen had a molecular weight of about 40 kDa (FIG. 9). In contrast, the PCR-based assay performed by an independent veterinary diagnostic laboratory did not distinguish between PLHV-1, -2, and -3. These results further demonstrate the sensitivity of the novel serology western blot assay over the methods used in the art for the detection of PLHV-1, PLHV-2, and PLHV-3.

In some embodiments, the described PCMV- and PLHV-specific automated serology assays are based on an optimized Wes™ protocol. The optimized Wes™ protocol comprises processes (e.g., incubation timing), reagents, dilutions, and antigen concentrations that are specific to porcine serum. The same protocol can be used on other size based automated western blot systems like Abby™ and Jess™. The PCMV- and PLHV-specific automated serology assays disclosed herein allowed for reliable detection of anti-PCMV and anti-PLHV antibodies (e.g., anti-IgG antibodies) in any subject.

In some embodiments, the assays described herein can process at least about 24 samples with a turnaround time of 4-5 hours to produce final results when compared to at least 8-24 hours for existing methods. In some embodiments, the turnaround time can be longer than a traditional Wes™ protocol. The novel assays disclosed herein can be used to detect anti-PCMV and anti-PLHV antibodies (e.g., anti-IgG antibodies) at various life stages of any subject (e.g., pigs). For example, the methods disclosed herein can detect maternal anti-PCMV and/or anti-PLHV antibodies. In some embodiments, the methods disclosed herein can detect anti-PCMV and/or anti-PLHV antibodies present in newborn piglets. In some embodiments, the methods disclosed herein can detect anti-PCMV and/or anti-PLHV antibodies in potentially infected young piglets (e.g., about 2-3 months old). In some embodiments, the methods disclosed herein can detect anti-PCMV and/or anti-PLHV antibodies in potentially infected donor pigs destined for xenotransplantation (e.g., >4 months old). The methods disclosed herein can also detect anti-PCMV and/or anti-PLHV antibodies in potentially infected young sows (e.g., >7 months old). Alternatively, the methods disclosed herein can detect anti-PCMV and/or anti-PLHV antibodies in potentially infected boars (e.g., >12 months old) and/or mature breeding sows (e.g., >12 months old).

In some embodiments, the novel assays described herein use porcine serum, the application of specific serum dilutions, antigen concentrations, and unique optimization to the Wes™ protocols to allow for consistent and reliable detection of PCMV or PLHV antibodies (e.g., IgG antibodies). The combination of the Simple WES™ or similar like Abby™, Jess™ and the novel antigen designs, and the process (e.g., increased incubation time) provide a novel serology system that can be run at any Veterinary Diagnostic laboratory (VDL) under Good Laboratory Practice (GLP) conditions.

Accordingly, one aspect of the present disclosure provides a method for detecting a porcine cytomegalovirus (PCMV) or a porcine lymphotropic herpesvirus (PLHV) in a biological sample from a subject, the method comprising obtaining a serum sample from the subject, diluting the serum sample with a diluent, contacting the diluted serum sample with a PCMV antigen or a PLHV antigen for a time period sufficient to allow binding between the PCMV antigen or PLHV antigen and a corresponding antibody in the diluted serum sample, detecting the presence or absence of an antibody from the diluted serum sample that reacts with the PCMV antigen or PLHV antigen using western blot analysis (e.g., an automated western blot system; or an automated capillary electrophoresis (CE) western, such as e.g., Simple Western™, or ProteinSimple's Jess™ or Wes™).

Another aspect of the present disclosure provides a method for detecting an anti-porcine cytomegalovirus (PCMV) antibody or an anti-porcine lymphotropic herpesvirus (PLHV) antibody in a biological sample from a subject. The method can comprise introducing into one or more capillaries of a microfluidic device one or more engineered PCMV antigens or one or more engineered PLHV antigens; a biological sample from the subject, one or more anti-immunoglobulin antibodies, and one or more chemiluminescent molecules; separating the one or more engineered PCMV antigens or the one or more engineered PLHV antigens electrophoretically; immobilizing the one or more electrophoretically separated engineered PCMV antigens or PLHV antigens on a capillary wall; contacting the one or more immobilized engineered PCMV antigens or PLHV antigens with the biological sample from the subject; incubating the one or more immobilized engineered PCMV antigens or PLHV antigens with the biological sample for about 10 minutes to about 120 minutes; and detecting the binding of the one or more engineered PCMV antigens or PLHV antigens to the biological sample by immunodetection and/or chemiluminescent detection. The presence of an immunodetection signal and/or a chemiluminescent signal can indicate the presence of one or more anti-PCMV antibodies or anti-PLHV antibodies in the biological sample.

In another aspect, the present disclosure provides an automated serology method for detecting an anti-porcine cytomegalovirus (PCMV) antibody or an anti-porcine lymphotropic herpesvirus (PLHV) in a porcine animal, the method comprising contacting one or more engineered PCMV antigens or PLHV antigens with a biological sample from the porcine animal; incubating the one or more engineered PCMV antigens or PLHV antigens with the biological sample for about 10 minutes to about 120 minutes; and detecting the binding of the one or more engineered PCMV antigens or PLHV antigens to the biological sample by immunodetection and/or chemiluminescent detection.

In any embodiments disclosed herein, the subject can be a porcine animal, a primate, a non-human primate, or a human. The porcine animal can be a wild-type porcine animal, or a transgenic porcine animal. In some embodiments, the human was transplanted with a cell, a tissue, or an organ derived from a porcine animal (e.g., a wild-type or transgenic). In some embodiments, the porcine lymphotropic herpesvirus is a porcine lymphotropic herpesvirus-1 (PLHV-1), porcine lymphotropic herpesvirus-2 (PLHV-2), and/or a porcine lymphotropic herpesvirus-3 (PLHV-3).

One of the advantages of an automated serology western blot assay described herein in relation to a xenotransplant is that it is serum/blood based. Thus, one can use the assay described herein on a sample (e.g., blood/serum) that is readily available/procured from a donor animal pre-transplant. This is especially important for detection of a latent virus that may be hiding in a lymph node or other hard to biopsy tissue like a spleen that cannot be readily obtained prior to organ procurement and subsequent transplant.

Accordingly, the novel serology assay for detection of porcine viruses disclosed herein can be applied to the detection of any virus. In some embodiments, the methods disclosed herein can be used to detect antibodies to any porcine virus, any zoonotic porcine virus, or any latent porcine virus. In that embodiment, the porcine virus can be of concern for xenotransplantation. The methods disclosed herein are particularly important for this type of assay as a pre-transplant screen due to the type of sample (serum/blood) used for detection. The methods described herein are more sensitive than PCR screening assays that are currently used in the art. Thus, the methods disclosed herein can be used as diagnostic and prognostic methods, thereby allowing a person of skill in the art to exclude a latently-infected donor animal prior to or in advance of a future transplant rather than peri-transplant.

### II. Definitions

Unless defined otherwise, all technical and scientific terms used herein generally have the same meaning as commonly understood by one of ordinary skill in the art to which this technology belongs.

As used herein, the singular forms “a,” “an” and “the” include plural referents unless the context clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “About.” The term “About” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “About” means either within plus or minus 10% of the provided value, or rounded to the nearest significant figure, in all cases inclusive of the provided value. In some embodiments, the term “about” indicates the designated value ±up to 10%, up to ±5%, or up to ±1%.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Headings, for example, (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, the term “Analyte” means a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

As used herein, the term “Animal” refers to a mammal. The animals of the present disclosure can be “genetically modified” or “transgenic.” A transgenic animal can be a genetically modified animal comprising one or more exogenous transgenes, or other foreign DNA, added or incorporated, or one or more endogenous genes modified, including, targeted, recombined, interrupted, deleted, disrupted, replaced, suppressed, enhanced, or otherwise altered, to mediate a genotypic or phenotypic effect in at least one cell of the animal and typically into at least one germ line cell of the animal. The animal may have the transgene integrated on one allele of its genome (heterozygous transgenic). Alternatively, the animal may have the transgene on two alleles (homozygous transgenic).

As used herein, the term “Electrophoresis” refers to the movement of suspended or dissolved molecules through a fluid or gel under the action of an electromotive force applied to electrodes in contact with a fluid.

As used herein, the term “Immobilizing” refers to substantially reducing or eliminating the motion of molecules in a fluid path. The immobilization can be via covalent bonds or non-covalent means, such as by hydrophobic or ionic interaction. In some embodiments, the resolved analytes (e.g., anti-PCMV or anti-PLHV antibodies) present in the sample are immobilized in the fluid path by isoelectric focusing (e.g., according to the Wes™ system specification).

As used herein, the term “Sample” means a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

A sample can be any substance containing or presumed to contain one or more analytes of interest (e.g., antibodies, in particular those to a PCMV or PLHV). The sample can be of natural or synthetic origin and can be obtained by any means known to those of skill in the art. Samples can also be synthetic and include, but are not limited to, in vitro cell culture constituents including, but not limited to, conditioned medium, recombinant cells, and cell components. The sample can be a tissue, a cell, an organ, or a fluid isolated from a subject. The fluid may include, but not be limited to, plasma, serum, whole blood, cerebrospinal fluid (CSF), semen, amniotic fluid, lymph fluid, synovial fluid, urine, or tears.

In some embodiments of the methods disclosed herein, the sample can be a blood product used to transfuse or treat. The sample can be any fluid found within the body of an organism that is capable of containing components of PCMV or PLHV, or an immune system component that may react to one or more PCMV or PLHV antigens. For example, the immune system component may be an antibody that specifically binds to a PCMV or PLHV antigen.

In some embodiments of the methods disclosed herein, the sample is a body fluid, which may include, for example, blood, plasma, serum, urine, saliva, tears, synovial fluid, and/or lymphatic fluid. In some embodiments, the sample is blood or whole blood. In some embodiments, the sample comprises a plasma or a serum. The sample can be a serum.

Accordingly, in some embodiments of the methods disclosed herein, the biological sample is selected from the group consisting of whole blood, serum, plasma, urine, seminal fluid, cerebrospinal fluid, and saliva. Alternatively, the biological sample is a whole blood, or a serum.

As used herein, the term “Subject” means any organism including, without limitation, a mammal. For example, the mammal can be a farm animal, a livestock animal, a pet, a primate, or a non-human primate. In some embodiments, the mammal can be a human, a rhesus, a cynomolgus, a dog, a cat, a mouse, a rat, a fox, a deer, a ferret, a guinea pig, a rabbit, a pig, a goat, cattle, a baboon, a monkey, or a horse. In a preferred embodiment, the subject is a human being, or a pig (e.g., porcine animal). In some embodiments, the mammal is a porcine sow and has given birth at least one time. In certain embodiments, the mammal is a non-human primate (e.g., a monkey or a baboon).

As used herein, the terms “Porcine,” “Porcine animal,” “pig,” and “swine” are generic terms referring to the same type of animal without regard to gender, size, or breed.

As used herein, the term “Operably linked” or “Conjugated” or “Fusion” means that, in relation to an engineered antigen sequence described herein, there are one or more sequences at the N- or C-terminus that, when transcribed and translated, create additional polypeptides in association with the enzyme amino acid sequence, thereby creating a conjugation or a fusion of one or more polypeptides from one expression vector.

As used herein, the term “Antibody” refers to an immunoglobulin molecule, which specifically binds with an antigen. Antibodies can be intact immunoglobulins derived from natural sources or from recombinant sources. Antibodies e can be immunoreactive portions of intact immunoglobulins. Antibodies are typically tetramers of immunoglobulin molecules. The antibodies of the present disclosure may exist in a variety of forms including, for example, polyclonal antibodies, monoclonal antibodies, Fv, Fab and F (ab) 2, as well as, single chain antibodies (scFv), and humanized antibodies. In some embodiments, an antibody refers to such assemblies (e.g., intact antibody molecules, immunoadhesins, or variants thereof) which have significant known specific immunoreactive activity to an antigen of interest (e.g., a virus associated antigen). Antibodies and immunoglobulins comprise light and heavy chains, with or without an interchain covalent linkage between them. Basic immunoglobulin structures in vertebrate systems are relatively well understood.

As used herein, the term “Antigen” or “Ag” is defined as a molecule that provokes an immune response. For example, a macromolecule or a protein expressed by a PCMV or a PLHV. This immune response may involve either antibody production, or the activation of specific immunologically-competent cells, or both. The skilled artisan will understand that any macromolecules, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene (e.g., a PCMV gene or a PLHV gene) and that these nucleotide sequences can be arranged in various combinations to elicit a desired immune response or binding property. Moreover, the skilled artisan will understand that an antigen need not be encoded by a “gene” at all. The antigen can be generated, synthesized, or can be derived from a biological sample (e.g., the engineered PCMV or PLHV antigens). Such a biological sample can include, but is not limited to a tissue sample, a tumor sample, a xenotransplant, a cell or a biological fluid.

As used herein, the term “Immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as, saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing release of mediators from mast cells and basophils upon exposure to allergen. In some embodiments of the present disclosure, the immunoglobulin is IgG or IgM. In some embodiments, the immunoglobulin is IgG.

As used herein, the term “Organ” refers to a collection of tissues joined in a structural unit to serve a common function. The organ may be a solid organ. Solid organs are internal organs that have a firm tissue consistency and are neither hollow (such as the organs of the gastrointestinal tract) nor liquid (such as blood). Examples of solid organs include the heart, kidney, liver, lungs, pancreas, spleen, and adrenal glands.

As used herein, the term “Primate” refers to various mammals of the order primates, which consists of lemurs, lorises, tarsiers, New World monkeys, Old World monkeys, apes, and humans. A primate is characterized by nails on the hands and feet, a short snout, and a large brain. In certain embodiments, the primate is a non-human primate. In other embodiments, the primate is a human.

As used herein, the terms “Detection,” “Detect,” and “Detecting” mean to discover the presence or existence of one or more analytes (e.g., an anti-PCMV or an anti-PLHV antibody)

As used herein, the terms “Identification,” “Identify,” and “Identifying” mean to recognize exposure to a specific pathogen or agent in sample from a subject.

As used herein, the term “Incubating” encompasses maintaining or providing conditions favorable for or conducive to a desired reaction such as, but not limited to, providing a reaction component or reagent. It is recognized that conditions favorable for or conducive to one reaction may differ from conditions favorable for another reaction. Suitable conditions for different reactions and reaction types are known in the art.

As used herein, the term “Contacting” means mixing, admixing, or incubating one or more components for the purpose of stimulating an interaction and/or binding of two or more of the components. In particular, contacting can also refer to bringing one or more components in closed proximity to trigger an interaction (e.g., binding) between the two or more components.

As used herein, the term “Identical” in the context of two nucleic acids or polypeptide sequences refers to the residues in the two sequences that are the same when aligned for maximum correspondence, as measured using a sequence comparison algorithms. Sequence comparison algorithms are known to those of skill in the art (see e.g., ebi.ac.uk/Tools/msa/clustalo/).

As used herein, the term “Sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using the publicly available computer software, such as Clustal Omega (Soding, J., Bioinformatics 21, 951-960 (2005)), T-coffee (Notre dame et al., J. Mol. Biol. 302, 205-217 (2000)), Kalign (Lassmann and Sonnhammer, BMC Bioinformatics, 6 (298) (2005)) and MAFFT (Katoh and Standley, Molecular Biology and Evolution, 30 (4) 772-780 (2013)). When using such softwares, the default parameters (e.g., for gap penalty and extension penalty), are preferably used.

### III. PCMV Serology Assay

Porcine Cytomegalovirus Infection (PCMV) is caused by a herpes virus found in tissues throughout the body including the nose of newborn piglets where it causes inflammation (rhinitis). PCMV is present throughout the world and exists in most pig populations tested to date. However, most infections are sub-clinical. At least so far, a clinical disease is rare. Serology analyses of several populations of pigs indicated that over 90% of herds have been exposed to infection. The rhinitis produced by this virus is uncommon and occurs mostly in newborn pigs. In most herds, the infection is insignificant and apart from sometimes causing a mild sneeze has no major effect on the health of the pig.

Regardless of the insignificant nature of the PCMV infection in pigs, for xenotransplantation, the donor pig must be screened for a detailed panel of potential zoonotic microbes prior to the use of their organs for non-human primate (NHP) or a human transplantation. PCMV, for example, is quite challenging because it can cross the placenta in pregnant sows; and PCMV can remain in a latent stage of infection that eludes detection by standard screening methods (e.g., polymerase-chain-reaction (PCR) assays). Thus, the methods of the present disclosure were developed to enable a person of skill in the art to perform high throughput serology screening to diagnose multiple infections in a subject (e.g., a human or a porcine animal) at all PCMV infection stages.

Accordingly, the novel serology assays for detection of porcine viruses disclosed herein can be applied to the detection of any virus. In some embodiments, the methods disclosed herein can be used to detect antibodies to any porcine virus, any zoonotic porcine virus, or any latent porcine virus. In that embodiment, the porcine virus can be of concern for xenotransplantation. The methods disclosed herein are particularly important for this type of assay as a pre-transplant screen due to the type of sample (serum/blood) used for detection. The methods described herein are more sensitive than PCR screening assays that are currently used in the art. The methods disclosed herein can be used as diagnostic and prognostic methods, thereby allowing a person of skill in the art to exclude a latently-infected donor animal prior to or in advance of a future transplant rather than peri-transplant.

### Optimized Methods for PCMV Antibody Detection

The present disclosure provides an optimization of Wes™ for PCMV antibody detection in a subject suspected of having PCMV infection, preferably a latent PCMV infection. Wes™ is an automated western blot method that can generate similar results as those obtained using a traditional western blot assay. See e.g., bio-techne.com/p/simple-western/wes_004-600. Western blot assay is the gold standard for protein detection and characterization. However, Wes™ is an improvement on this gold standard because it is a gel-free, blot-free, hands-free alternative assay for protein sizing and quantitative immunodetection. Wes™ is an automated system that integrates and automates all manual operations associated with western blotting. The entire Wes™ process is also faster, simpler, more sensitive, more accurate, and more automatable than traditional western blot analytical techniques. For example, using Wes™, analytes can be analyzed within about 50 minutes compared to up to 24 hours with a traditional western blot. With Wes™, the initial separation of cellular materials can take about 5 minutes or less. The subsequent immobilization can take about 2 minutes or less. The detection agents can be linked to the separated sample within 10 minutes or less of the commencement of separation, and the one or more analytes can be analyzed within 30 minutes of the separating step. See e.g., U.S. Pat. No. 7,935,489. As such, the present disclosure provides rapid and sensitive serology assays to determine if a subject is infected with PCMV using a PCMV serology assay based on an optimization of Wes™ for PCMV. While the assays described herein can be performed using a traditional western blot assay, but the Wes™ method will generally be preferred.

While Wes™ is exemplified, the assays described herein can be used with any automated capillary electrophoresis (CE) western system (e.g., Simple Western™, or ProteinSimple's Jess™ or Wes™) to identify, characterize, and quantify the target anti-viral antigen antibody (e.g., an anti-PCMV antibody or an anti-PLHV antibody). The CE western comprises a protein size separation step (e.g., a size distribution profiling), a protein immobilization step, and a protein detection step using fluorescent detection and/or chemiluminescent detection. As such, the CE western technique can be a sensitive anti-viral antigen characterization, qualification, and quantification method with enhanced resolution and size separation range.

In addition, CE can be more sensitive than an enzyme-linked immunosorbent assay (ELISA) for the quantitative analysis of targets proteins in a serum sample from immunized animals. While an ELISA can only provide protein binding information, CE western techniques can provide additional information about the target protein, such as molecular weight estimation (e.g., as size, and size distributions) and protein intactness (e.g., isomers, fragments, and aggregates).

In some embodiments, the CE is Jess™ (or equivalent thereof). Accordingly, in step 4 of FIG. 1, the sample can be prepared for Jess™ (or equivalent thereof) and can be loaded into a Jess™ plate. Specifically, one or more serum samples comprising one or more viral antigens (e.g., PCMV or PLHV), can be placed in a single well along with a serum diluent or a buffer, a primary antibody that targets the one or more viral antigens (e.g., PCMV or PLHV) and/or a secondary antibody conjugated to a chemiluminescent or a fluorescent molecule, and Jess™ reagents (Standard Equipment for Western Assays). The chemiluminescent or the fluorescent molecule can be obtained from any known supplier. Jess™ reagents can be obtained from the manufacturer.

Accordingly, one aspect of the present disclosure provides a method for detecting a porcine cytomegalovirus (PCMV) in a biological sample from a subject, the method comprising, consisting essentially of, or consisting of obtaining a serum sample from the subject, diluting the serum sample with a diluent, contacting the diluted serum sample with a PCMV antigen for a time period sufficient to allow binding between the PCMV antigen and a corresponding antibody in the diluted serum sample, detecting the presence or absence of an antibody from the diluted serum sample that reacts with the PCMV antigen using western blot analysis, an automated capillary electrophoresis (CE) western, including Wes™, Jess™ or equivalent thereof.

Another aspect of the present disclosure provides a method for detecting an anti-porcine cytomegalovirus (PCMV) antibody in a biological sample from a subject, the method comprising: introducing into one or more capillaries of a microfluidic device one or more engineered PCMV antigens; a biological sample from the subject, one or more anti-immunoglobulin antibodies, and one or more chemiluminescent molecules. Following this initial step, the one or more engineered PCMV antigens are electrophoretically separated followed by the immobilization of the one or more electrophoretically separated engineered PCMV antigens on a capillary wall. Once immobilized, the one or more immobilized engineered PCMV antigens are contacted with the biological sample from the subject. Then, the one or more immobilized engineered PCMV antigens with the biological sample are incubated for about 10 minutes to about 120 minutes.

In some embodiments, the one or more engineered PCMV antigens and the biological sample are incubated for about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 100 minutes, about 105 minutes, about 110 minutes, about 115 minutes, or about 120 minutes. This incubation period is much longer than the recommended immobilization step of about 2 minutes or less.

Lastly, the binding of the one or more engineered PCMV antigens to the biological sample is detected by immunodetection and/or chemiluminescent detection. In some embodiments, the presence of an immunodetection signal and/or a chemiluminescent signal can indicate the presence of one or more anti-PCMV antibodies in the biological sample. In some embodiments, the immunodetection signal is a fluorescent signal.

Yet another aspect of the present disclosure provides an automated serology method for detecting an anti-porcine cytomegalovirus (PCMV) antibody in a porcine animal, the method comprising, consisting essentially of, or consisting of contacting one or more engineered PCMV antigens with a biological sample from the porcine animal. Followed by the incubation of the one or more engineered PCMV antigens with the biological sample for about 10 minutes to about 120 minutes, and detecting the binding of the one or more engineered PCMV antigens to the biological sample by immunodetection and/or chemiluminescent detection.

Embodiments of the methods described herein are illustrated in FIG. 1. Step 1 can begin with the collection of whole blood from a subject. In some embodiments, about 1-2 mL are collected. The subject can be a human or a porcine animal as defined herein. In step 2, serum is separated from whole blood by centrifugation using any methods known to those of skill in the art. In step 3, the separated serum from the subject can be aliquoted into vessels for analysis (e.g., Eppendorf tubes). In step 4, the sample is prepared for Wes™, Jess™ (or equivalent thereof) and can be loaded into a Wes™ plate. Specifically, one or more engineered antigens contemplated by the present invention (e.g., an engineered PCMV antigen; an engineered PLHV antigen) can be placed in a single well along with the pig serum diluted in serum diluent, a rabbit anti-pig IgG HRP secondary antibody, which can be obtained from any known supplier; and Simple Wes™ reagents or Jess™ reagents (Standard Equipment for western Assays), which may be obtained from the manufacturer. Thus, the serology assay disclosed herein can be an automated capillary-based immunoassay system (e.g., Wes™, a ProteinSimple instrument). The diluent can be a buffer. The diluent can be an instrument-specific buffer produced by a manufacturer. In step 5, the Wes™ plate can be loaded in Simple Wes™ instrument. In step 6, the results generated by the Simple Wes™ instrument can be analyzed using Compass software. Thus, the protein band images and quantitative results can be generated by Compass software after running one or more samples on the Wes™ or Jess™ capillary electrophoresis system (ProteinSimple) and/or using chromatogram output data.

In some embodiments, the one or more engineered PCMV antigens are electrophoretically separated by weight and immobilized on a solid support. The solid support can be a capillary wall of a microfluidic device. The method disclosed herein can be performed in a closed-loop automated capillary-based immunoassay system (e.g., Simple Wes™ instrument).

### Immunodetection

In some embodiments of the methods disclosed herein, the methods can comprise detecting the binding of the one or more engineered PCMV antigens present in the biological sample by immunodetection and/or chemiluminescent detection. In those embodiments, the presence of an immunodetection signal and/or a chemiluminescent signal indicates the presence of one or more analytes (e.g., anti-PCMV or anti-PLHV antibodies) in the biological sample.

The method comprises a detection agent that is capable of binding to or interacting with the analyte to be detected in the biological sample. Non-limiting examples of detection agents include proteins, peptides, antibodies, enzyme substrates, transition state analogs, cofactors, nucleotides, polynucleotides, aptamers, lectins, small molecules, ligands, inhibitors, drugs, and other biomolecules as well as non-biomolecules capable of binding the analyte to be detected. In some embodiments, the detection agent is an antibody.

In some embodiments, the detecting the binding of the one or more engineered PCVM or PLHV antigens comprises a dual antibody CE western. The dual antibody CE western can comprise: (a) a primary antibody that targets the one or more engineered PCVM or PLHV antigens; (b) a secondary antibody that targets the primary antibody; and (c) a chemiluminescent or a fluorescent molecule substrate. In some embodiments, the secondary antibody that targets the primary antibody is operably linked to a chemiluminescent or a fluorescent molecule.

The antibody can be an IgM and/or an IgG antibody. In some embodiments, the analyte to be detected in the biological sample is one or more anti-PCMV antibodies. Thus, in some embodiments of the methods disclosed herein, the anti-PCMV antibody is an IgM or an IgG antibody.

Contacting the detection agent with the one or more analytes in the biological sample (e.g., the one or more anti-PCMV antibodies) can be by any method known in the art. However, the chosen method should be compatible with the methods described herein (e.g., Wes™, Jess™). The detection agents can comprise any organic or inorganic molecule capable of binding to interact with the analyte to be detected (e.g., the one or more anti-PCMV or anti-PLHV antibodies).

In some embodiments, the detection agents can comprise one or more label moieties. In some embodiments, the detection agent can comprise two or more label moieties. In that embodiment, each label moiety can be the same or different.

In some embodiments, the label moiety may comprise an immunodetection label or a chemiluminescent label (e.g., immunodetection and/or chemiluminescent detection). The chemiluminescent label can comprise any entity that provides a light signal and that can be used in accordance with the methods and devices described herein. A wide variety of such chemiluminescent labels are known in the art. See, e.g., U.S. Pat. Nos. 6,689,576, 6,395,503, 6,087,188, 6,287,767, 6,165,800, and 6,126,870.

Suitable labels include enzymes that are capable of reacting with a chemiluminescent substrate to induce a photon emission by chemiluminescence. The labels may include peroxidase, beta-galactosidase, phosphatase, or others for which a chemiluminescent substrate is available. In some embodiments, the chemiluminescent label can be selected from any of a variety of classes of luminol, or isoluminol. In some embodiments, the detection agents comprise one or more chemiluminescent substrates.

Chemiluminescent detection can be a more sensitive detection method for detecting target molecules that may be in low abundance in a biological sample. For example, chemiluminescent detection can maximize immunodetection output by detecting protein that are present in the sample at picogram concentration.

In some embodiments, the method described herein detects a nanogram or a picogram (pg) of the anti-PCMN antibody or anti-PLHV antibody in the biological sample. In some embodiments, the method detects at least about 0.0001 ng-1.0 ng, at least about 0.0001 ng-0.1 ng.

In some embodiments, the method detects at least about 0.1 pg-1.0 pg, at least about 0.5 pg-2.5, at least about 0.1 pg-10 pg, at least about 5 pg-50 pg, at least about 10 pg-100 pg, or more than 100 pg of the anti-PCMN antibody or anti-PLHV antibody in the biological sample. In some embodiments, the method detects at least about 0.1 pg, at least about 0.2 pg, at least about 0.3 pg, at least about 0.4 pg, at least about 0.5 pg, at least about 0.6 pg, at least about 0.7 pg, at least about 0.8 pg, at least about 0.9 pg, at least about 1.0 pg, at least about 1.1 pg, at least about 1.5 pg, at least about 5.0 pg, at least about 10 pg, at least about 20 pg, at least about 30 pg, at least about 40 pg, at least about 50 pg, at least about 60 pg, at least about 70 pg, at least about 80 pg, at least about 90 pg, or at least about 100 pg of the anti-PCMN antibody or anti-PLHV antibody in the biological sample.

The label moiety can also comprise a bioluminescent compound. As used herein, bioluminescence is a type of chemiluminescence found in biological systems in which a catalytic protein increases the efficiency of the chemiluminescent reaction. The presence of a bioluminescent compound is determined by detecting the presence of luminescence. Suitable bioluminescent compounds can include luciferin, luciferase and aequorin.

In some embodiments, the label moiety comprises a fluorescent dye. The fluorescent dye comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, non-limiting examples include xanthenes, rhodamines, fluoresceins, cyanines, phthalocyanines, squaraines, bodipy dyes, coumarins, oxazines, and carbopyronines. In some embodiments, for example, where detection agents contain fluorophores, such as fluorescent dyes, their fluorescence is detected by exciting them with an appropriate light source and monitoring their fluorescence by a detector sensitive to their characteristic fluorescence emission wavelength. In some embodiments, the detection agents comprise fluorescent dye labeled antibodies.

In some embodiments, the immunodetection and/or chemiluminescent detection comprises a Horseradish Peroxidase (HRP) conjugated secondary antibody, streptavidin-HRP, luminol-S, peroxide, LacZ, biotin, or luciferase. In some embodiments, the detection agents comprise chemiluminescent labeled antibodies. In some embodiments, the immunodetection and/or chemiluminescent detection comprises a horseradish peroxidase (HRP) conjugated anti-IgG antibody or anti-IgM antibody.

Analyte detection can include detecting the presence or absence, measurement, and/or characterization of an analyte (e.g., the one or more anti-PCMV or anti-PLHV antibodies). Typically, an analyte (e.g., the one or more anti-PCMV antibodies or anti-PLHV) is detected by detecting a signal from a label. Such detection can include, but is not limited to, detecting isotopic labels, immune labels, optical dyes, enzymes, particles, and combinations thereof, such as chemiluminescent labeled antibodies and fluorescent labeled antibodies.

Moreover, the methods disclosed herein allow a person of skill in the art to monitor a signal in real time. The real time monitoring can allow the user to rapidly determine whether an analyte is present in the sample, and/or the amount or activity of the analyte. In some embodiments, the signal can be measured from at least two different time points. In some embodiments, the signal can be monitored continuously or at several selected time points. Alternatively, the signal can be measured at a predetermined end-point. For example, the signal can be measured after a certain amount of time. Alternatively, the signal can be compared against a control signal (e.g., sample without analyte; or a sample without biological material), threshold signal, or standard curve. The amount of the signal generated may not be critical and can vary over a broad range. The only requirement may be for the signal to be measurable by the detection system being used. In some embodiments, a signal can be at least 2-fold greater than the background signal (e.g., control signal; sample without analyte; or a sample without biological material). In some embodiments, a signal can be 2 to 10-fold greater than the background. In some embodiments, a signal can be 10-fold greater than the background.

### Subjects

In certain embodiments, the mammal is a porcine animal. In some embodiments of the methods disclosed herein, the porcine animal is about 60-323 days old. For example, the porcine animal can be about 30 days, about 40 days, about 50 days, about 60 days, about 70 days, about 80 days, about 90 days, about 100 days, about 110 days, about 115 days, about 116 days, about 150 days, about 180 days, about 200 days, about 215 days, about 230 days, about 238 days, about 215 days, about 238 days, about 240 days, about 317 days, about 323 days, about 340 days, or about 350 days old.

The porcine animal may be of any size. For example, the porcine animal may weigh from about 10 pounds to about 500 pounds. In some embodiments, the porcine animal may weigh more than 500 pounds. The weight of the porcine animal may for example depend on the xenotransplant recipient's weight and size. The porcine animal can be a wild-type porcine animal, or a transgenic porcine animal.

The novel automated serology assays described herein can include the use of porcine serum or a serum from a subject transplanted with a porcine cell, tissue or organ, the application of specific dilutions and antigen concentrations, and unique modifications to the Wes™ protocols.

In some embodiments of the methods disclosed herein, the subject is suspected of having a latent PCMV or PLHV infection, a lytic PCMV or PLHV infection, or active viremic PCMV or PLHV infection. The PCMV or PLHV infection can be a latent infection when the presence of PCMV or PLHV in the body does not result in the manifestation of pathological symptoms. This period is akin to an incubation period. Generally, during a latent infection a PCR-test will provide a negative result. Latent infection can sometimes occur when an infectious agent survives in the patient for a variable time following an infectious disease.

In some embodiments, the PCMV or PLHV infection can be a latent infection when the presence of the one or more anti-PCMV or anti-PLHV antibodies is detected in the biological sample and a standard PCR assay using the same one or more engineered PCMV or PLHV antigens is negative. In some embodiments, the PCMV or PLHV infection is a latent infection when the presence of the one or more anti-PCMV or anti-PLHV antibodies are detected in the biological sample with a HRP conjugated anti-IgG antibody but not with a HRP conjugated anti-IgM antibody.

### Sample

In some embodiments, the biological sample can be selected from the group consisting of whole blood, serum, plasma, urine, seminal fluid, cerebrospinal fluid and saliva. The biological sample can also be whole blood, or a serum.

In some embodiments, the biological sample is diluted to about 1:20; about 1:40; about 1:50; about 1:75, about 1:100; about 1:150; about 1:120; about 1:250; about 1:300; about 1:350; about 1:375; about 1:400; about 1:500; about 1:750; about 1:800; about 1:1000; about 1:1100; or about 1:1200. In some embodiments, the biological sample is diluted at about 1:75, about 1:200, or about 1:300 (FIGS. 2-4). In some embodiments, the biological sample is diluted at about 1:75.

In some embodiments, the one or more engineered PCMV antigens or the one or more engineered PLHV antigens can be used at a concentration of about 0.1 ug; about 0.125 ug; about 0.150 ug; about 0.2 ug; about 0.225 ug; about 0.25 ug; about 0.3 ug; about 0.315 ug; about 0.325 ug; about 0.350 ug; about 0.4 ug; about 0.415 ug; about 0.425 ug; about 0.450 ug; about 0.5 ug; about 0.515 ug; about 0.52 ug; about 0.55 ug; or about 0.6 ug.

### Engineered PCMV Antigens

The methods disclosed herein are based on the design of recombinant PCMV antigens. Many studies have shown that human sera can react with a broad spectrum of human CMV proteins from either purified virus or virus-infected cells. In human, it has been observed that the majority of the anti-glycoprotein response is directed toward glycoprotein B of the human CMV. Foust et al., J. Virol. 86 (13): 7444-7447 (2012). However, the major neutralizing antibody responses observed against human CMV proteins appear to be directed to non-gB glycoprotein, such as the gH/gL/UL128/UL130/UL131 complex. It is however important to note that many human CMV genes are not conserved in the PCMV genome. In fact, HCMV is not related to PCMV at the molecular level. Following the molecular characterization of the PCMV genome, PCMV was re-designated as a roseolovirus by the International Committee on Taxonomy of Viruses. Gu et al., Virology 460-461:165-172 (2014). So far, no correlations between antibody reactivity against PCMV and HCMV have been reported. In addition, human CMV antibodies do not cross react with PCMV. Therefore, to fully capture the presence of PCMV in a biological sample, it is important to identify and design as many relevant antigens as possible.

Accordingly, in some embodiments of the methods disclosed herein, the one or more engineered PCMV antigens can comprise one or more engineered PCMV antigens selected from the group consisting of envelope glycoprotein B (gB), envelope glycoprotein H (gH), envelope glycoprotein L (gL), envelope glycoprotein M (gM), envelope glycoprotein N (gN), major tegument phosphoprotein1 (U54A); major tegument phosphoprotein2 (U54B); U100p.

In some embodiments, the one or more engineered PCMV antigens comprise glycoprotein B (gB). The one or more engineered PCMV antigens can comprise glycoprotein B gB, gH, and gL. In some embodiments, the one or more engineered PCMV antigens comprise gB, gM, and gN. In some embodiments, the one or more engineered PCMV antigens comprise gB, and a phosphoprotein pp65. In some embodiments, the one or more engineered PCMV antigens comprise gB, gH, gL, gM, gN, and U100(Q1). In some embodiments, the one or more engineered PCMV antigens comprise gB, gH, gL, gM, gN, U100(Q1), and a phosphoproteins pp65.

The one or more engineered PCMV antigens contemplated by the present disclosure can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 25, 26, 27, 28, 29, 30, 31, or 32.

In some embodiments, the one or more engineered PCMV antigens comprise the amino acid sequence of SEQ ID NO: 2, 3, 8, 9, 25, 26, 27, 28, 29, 30, 31, 32, or a combination thereof. In some embodiments, the one or more engineered PCMV antigens comprise the amino acid sequence of SEQ ID NO: 2, 3, 8, 9, 25, 26, 27, 28, 29, 30, or 31 and further comprise a tag. In some embodiments, the tag comprises the amino acid sequence set forth in SEQ ID NO: 33. In some embodiments, the one or more engineered tagged PCMV antigens comprise the amino acid sequence of SEQ ID NO: 32.

In some embodiments, the one or more engineered PCMV antigens comprise the amino acid sequence of SEQ ID NO: 2. In some embodiments, the one or more engineered PCMV antigens comprise the amino acid sequence of SEQ ID NO: 3 or SEQ ID NO: 32. In some embodiments, the one or more engineered PCMV antigens comprise the amino acid sequence of SEQ ID NO: 2, 3, 8, 9, 25, 26, 27, 28, 29, 30, 31, and 32.

ACM17140.1 glycoprotein B [Suid betaherpesvirus 2]
SEQ ID NO: 2
MTVSSRNLFRIVLLINYGYLVSSNSTTSTTSASESSVTTGSPGTVSSETA
TPEVSTAFVNITGNFSQEYTEASDDEKYPFRVCNMAVGTDLYRFDNYITC
NKYNTETQYSEGILLLFKKNIVPHTFFVRTYTKELSFQTTYRDVHVIYLV
DRPSYKVPVPVDEAGYINLNGQCFSAAEIRNQGINYRVYHKDDNTNNTMR
LYRLKFGSTINTRYISTPEFQFTYGTHWLYKSSSSINCIVTDTIGKSDYP
YENFILGTGESVEISPFFNGTSKEVFNEQMYYFSMKSNYTMLEKLDEPNG
PKKTIPTIAFLQKGDTLFSWEVKEQTNSHCKYTAWTKKHHALRADMTNSY
HFMMKDMTATMVTTKNTINLTGTEYECVKNDIEKYITDTFQSKYNNTHNK
TENYSVYETTTGLILVWQPIVRKSIKELKEFINETQHTRYKREIDGSDSL
VYASLQYMYDALREYINAGFAQIAEAWCEDQKRTNEVLSELAKINPSNVM
SVIYDKSLSAKLVGDAISVSSYVNVNQSTVKVHKDMRIYANGTANRETCF
SRPVVTFEFSNNNSVQQFTGQLGPRNEILLGTYRVEKCERNSVKVFFAGK
EAYFFYNYIYTKTVNISDINVVDTFIHLNIKPLENTDFEVLRMYSKNELV
QANIFDLESLLRDINSYRSALYNIESRIAPRKPDYVSGVDSFLHALGIGA
PGGLGAALGMATGAVTDFVTGIFSFFKNPFGGLFSMLFFVLLVFLIFSVY
YRQKNIYTNPVGALFPYANSSSGTAISNTHSYYETDNKQEFENDRKPDTS
NAVSEGSANKYSQEDAVCMLMAIKNLGDAYRRKNATKPSPSVLDKIRHLE
YQQLSTEDV

In some embodiments, the one or more engineered PCMV antigens comprise a fragment of the amino acid sequence of SEQ ID NO: 2. For example, the fragment can comprise the C-terminus of the glycoprotein B. In some embodiments, a fragment of the C-terminus of the glycoprotein B comprises the amino acid sequence of SEQ ID NO: 3. In some embodiments, a fragment of the C-terminus of the glycoprotein B comprises the amino acid sequence of SEQ ID NO: 32.

SEQ ID NO: 3
MNSSSGTAISNTHSYYETDNKQEFENDRKPDTSNAVSEGSANKYSQEDAVCMLMAI
KNLGDAYRRKNATKPSPSVL DKIRHLEYQQLSTED
SEQ ID NO: 8
MLIFSVYYRQKNIYTNPVGALFPYANSSSGTVISNTHSYYETNNKQEFENDRKPDTSN
AVSEGSANKYSQEDAVCMLMAIKNLGDAYRRKNATKPSPSVLDKIRHLEYQQLSTE
DV
AAL47542.1 glycoprotein B [Suid betaherpesvirus 2]; Porcine
cytomegalovirus strain (nucleotides 539-3118); glycoprotein B (gB)
SEQ ID NO: 9
MTVSSRNLFRIVLLINYGYLVSSNSTTSTTSASESSVTTGSPGTVSSETATPEVSTAFVN
ITGNFSQEYTEASDDEKYPFRVCNMAVGTDLYRFDNYITCNKYNTETQYSEGILLLF
KKNIVPHTFFVRTYTKELSFQTTYRDVHVIYLVDRSSYKVPVPVDEAGYINLNGQCFS
AAEIRNQGINYRVYHKDDNTNNTMRLYRLKFGSTINTRYISTPEFQFTYGTHWLYKS
SSSINCIVTDTLAKSDYPYENFILGTGESVEISPFFNGTSKEVFNEQMYYFSMKNNYT
MLEKLDEPNGPKKTIPTIAFLQKGDTLFSWEVKEQTNSHCKYTAWTKKHHALRADM
TNSYHFMMKDTTATMVTTKNTINLTGTEYECVKNDIEKYITDTFQNKYNNTHNKTE
NYSVYETTTGLILVWQPIVRKSIKELKEFINETQHTRYKREIDGSDSLVYASLQYMYD
ALREYINAGFAQIAEAWCEDQKRTNEVLSELAKISPSNVMSVIYDKSLSAKLVGDAIS
VSSCVNVNQSTVKVHKDMRIYANGTANRETCFSRPVVTFEFSNNNSVQQFTGQLGP
RNEILLGTYRVEKCERNSVKVFFAGKEAYFFYNYIYTKTVNISDINVVDTFIHLNIKPL
ENTDFEVLRMYSKNELAQANIFDLESLLRDINSYRSALYNIESRIAPRKPDYVSGVDSF
LHALGIGAPGGLGAALGMATGAVTDFLTGIFSFFKNPFGGLFSMLFFVLLVFLIFSVY
YRQKNIYTNPVGALFPYANSSSGTVISNTHSYYETNNKQEFENDRKPDTSNAVSEGS
ANKYSQEDAVCMLMAIKNLGDAYRRKNATKPSPSVLDKIRHLEYQQLSTEDV
C-terminus fragment-glycoprotein B (gB) (AF268039.2 nucleotides 2771-3118)
SEQ ID NO: 25
MFPYANSSSGTVISNTHSYYETNNKQEFENDRKPDTSNAVSEGSANKYSQEDAVCM
LMAIKNLGDAYRRKNATKPSPSVLDKIRHLEYQQLSTEDV
ACM17140.1 glycoprotein B
SEQ ID NO: 26
MFPYANSSSGTAISNTHSYYETDNKQEFENDRKPDTSNAVSEGSANKYSQEDAVCM
LMAIKNLGDAYRRKNATKPSPSVLDKIRHLEYQQLSTEDV
N-terminus fragment-glycoprotein B (gB) (AF268039.2 nucleotides 539-3118)
SEQ ID NO: 27
MTVSSRNLFRIVLLINYGYLVSSNSTTSTTSASESSVTTGSPGTVSSETATPEVSTAFVN
ITGNFSQEYTEASDDEKYPFRVCNMAVGTDLYRFDNYITCNKYNTETQYSEGILLLF
KKNIVPHTFFVRTYTKELSFQTTYRDVHVIYLVDRSS
ACM17140.1 glycoprotein B N-terminus fragment
SEQ ID NO: 28
MTVSSRNLFRIVLLINYGYLVSSNSTTSTTSASESSVTTGSPGTVSSETATPEVSTAFVN
ITGNFSQEYTEASDDEKYPFRVCNMAVGTDLYRFDNYITCNKYNTETQYSEGILLLF
KKNIVPHTFFVRTYTKELSFQTTYRDVHVIYLVDRPS
ACM17140.1 glycoprotein B-fragment
SEQ ID NO: 29
MTKTVNISDINVVDTFIHLNIKPLENTDFEVLRMYSKNELAQANIFDLESLLRDINSYR
SALYNIESRIAPRKPDYVSGVDSFLHALGIGAPGGLGAALGMATGAVTDFLTGIFSFF
KNPFGGLFSMLFFVLLVFLIFSVYYRQKNIYTNPVGAL
AAL47542.1 glycoprotein B-fragment
SEQ ID NO: 30
MTKTVNISDINVVDTFIHLNIKPLENTDFEVLRMYSKNELAQANIFDLESLLRDINSYR
SALYNIESRIAPRKPDYVSGVDSFLHALGIGAPGGLGAALGMATGAVTDFLTGIFSFF
KNPFGGLFSMLFFVLLVFLIFSVYYRQKNIYTNPVGAL
ACM17140.1 glycoprotein B-fragment (center)
SEQ ID NO: 31
MYKVPVPVDEAGYINLNGQCFSAAEIRNQGINYRVYHKDDNTNNTMRLYRLKFGST
INTRYISTPEFQFTYGTHWLYKSSSSINCIVTDTIGKSDYPYENFILGTGESVEISPFFNG
TSKEVFNEQMYYFSMKSNYTMLEKLDEPNGPKKTIPTIAFLQKGDTLFSWEVKEQTN
SHCKYTAWTKKHHALRADMTNSYHFMMKDMTATMVTTKNTINLTGTEYECVKND
IEKYITDTFQSKYNNTHNKTENYSVYETTTGLILVWQPIVRKSIKELKEFINETQHTRY
KREIDGSDSLVYASLQYMYDALREYINAGFAQIAEAWCEDQKRTNEVLSELAKINPS
NVMSVIYDKSLSAKLVGDAISVSSYVNVNQSTVKVHKDMRIYANGTANRETCFSRP
VVTFEFSNNNSVQQFTGQLGPRNEILLGTYRVEKCERNSVKVFFAGKEAYFFYNYIY
SEQIDNO: 32
MNSSSGTAISNTHSYYETDNKQEFENDRKPDTSNAVSEGSANKYSQEDAVCMLMAI
KNLGDAYRRKNATKPSPSVLDKIRHLEYQQLSTEDHHHHHHHHHH
In some embodiments of the methods disclosed herein, the one or more engineered
PCMV antigens are encoded by a polynucleotide sequence selected from SEQ ID NO: 1, 5, 6,
7, 10, 11, or a combination thereof.
Porcine cytomegalovirus glycoprotein B gene; GenBank: FJ595497.1
SEQ ID NO: 1
atgacagtgagcagtcggaatttattccggattgttttgttgattaactatggctatctcgtgtcatctaattctacgacatctacaacttcggc
atctgaatcatcggttaccaccggatctcccggcactgtcagttctgaaacagcgacaccggaagtttctacggcttttgtgaacattacc
ggaaatttttcacaggaatacactgaagcaagcgatgatgaaaaatatccttttcgtgtatgcaatatggccgttggaacagatctatata
gatttgataattatatcacttgtaataagtataatacggagactcagtattctgaaggtattttgttattgttcaagaaaaatatagttccgcac
actttttttgttaggacgtataccaaggaattgtcgtttcaaacgacgtatagagacgttcatgtcatctatcttgttgataggccttcatataa
agtgcccgttcctgtagatgaagctggatatataaatttgaatggtcaatgtttttccgctgccgagattagaaaccagggaataaattatc
gggtatatcataaagatgataatacaaacaatacaatgagattatatcgtttgaaatttggatctaccattaatactcgttacatatccacgc
ctgaatttcagttcacatatggaacccattggttatataaaagctcatcttctattaattgcatagtaactgatactattggtaaatctgattacc
catatgagaattttatactcggtacaggtgaatcggtggagatatcaccattttttaatggaacttctaaagaggtatttaatgaacagatgt
actatttttcaatgaagagcaattacaccatgttagaaaaattagacgaacctaatggaccaaagaaaactatccctacgattgcttttttac
aaaagggtgacacattgttttcatgggaggttaaggaacagactaattctcattgcaaatatacagcatggacaaaaaaacaccatgcat
tgcgagcagacatgacaaatagttatcattttatgatgaaggacatgacagcaacaatggtaacaacaaagaacactataaatcttacgg
gcaccgaatacgagtgtgtgaaaaatgatattgaaaaatatattacagatacgtttcagagcaaatataacaatacacacaacaaaacag
aaaactatagtgtttatgaaacaactacaggtctaatattagtttggcagcctattgttaggaaatcaattaaagagttgaaagaatttatca
atgaaacccaacatactcgttataaacgagaaattgatggaagtgactcacttgtttacgctagtttgcagtatatgtacgatgctttgaga
gagtatataaatgctggtttcgcccagatagctgaggcgtggtgcgaagatcagaaaagaaccaatgaagttctttccgaattagctaaa
ataaatccatctaatgtcatgtctgtaatctacgataaatcattatccgcaaaattagttggagatgcaatttccgtatcttcgtatgtcaatgt
aaatcagtctacggtgaaggtgcataaagatatgagaatatacgccaatgggacggcgaatagggagacgtgtttttctcgcccggtc
gttacgtttgaattttcgaataataattcagttcaacaatttacgggtcaattggggccgaggaatgagattctgttaggcacgtatagagt
ggaaaaatgtgaacgtaacagtgttaaagttttttttgcgggtaaggaggcgtattttttttacaattacatttacacgaaaacggttaatata
tctgatatcaatgtcgtagatacttttatccaccttaatataaagccgttagagaatacagactttgaggtattgcgaatgtattctaaaaatg
agttagtgcaggctaacatatttgatctggaaagtcttttgagagatattaattcatataggagtgcgctttataatattgaatctaggatagc
tccaaggaaaccagattacgtctcaggtgtggattcttttctgcatgctcttggtataggtgcacccggagggttgggtgccgctctgggt
atggcgacgggggcggtcacagatttcgtgactggcattttttccttctttaagaatccattcgggggacttttttccatgctgttttttgttttg
cttgttttcttgatattttccgtgtattacagacagaagaatatatatacaaacccggtaggtgcgttgtttccgtatgcaaatagttcttcggg
aactgcaatatctaatacgcattcctattatgagacagacaataaacaggagtttgagaatgacaggaaacctgatacgtccaatgccgt
ctctgagggcagtgccaataagtactctcaagaagatgccgtctgcatgttaatggctataaagaacctgggggatgcgtacaggcgg
aagaatgccacaaagcctagcccgagcgtattagataagatacgtcacttggaatatcagcagttatccaccgaggacgtgtga
AF268039.2 Porcine cytomegalovirus strain B6 ORF 40-like protein
gene; and glycoprotein B (gB) and DNA polymerase (pol) genes
SEQ ID NO: 4
ggccaaaagatttaccacctgttaatcggacccgtgatcacacatcacagagacacgtttccactaccgttcaatatagacatggcatatt
catgcgacaatgccaacatattgccacacgtaaaggagaacctcgtgaagtgcatagagagcaatatcggtcccggacagtggatgg
tgtcatcatacaatggtttttttgacttttcggattcaaatgacataaatgagatgcagagaaaaatgtggttgtatgtaaaagagctggttttt
ggcgtggccctatacaatgatatttttaatcagtctatacaagtgtacagagtcgatgaggtggatgagacgatggaaggtattattctga
catataactgtaactatccgttattgttaaatctaaaatctaagatatataagtctagcgatttgtatctgctgttctattgtatatttgactccgt
gaaggaaacgagtggcgagaccgtgagatgcgtacgtcgcacgcataaacgcgtatgtttgcggtctcttgtcagagatgacagtga
gcagtcggaatttattccggattgttttattgattaactatggatatctcgtgtcatctaattctacgacatctacaacttcggcatctgaatcat
cggttaccaccggatctcccggtactgtcagttctgaaacagcgacaccggaagtttctacggcttttgtgaacattaccggaaatttttc
acaggaatacactgaagcaagcgatgatgaaaaatatccttttcgtgtatgcaatatggccgttggaacagatctatatagatttgataatt
atatcacttgtaataagtataatacggagactcagtattctgaaggtattttgttattgtttaagaaaaatatagttccgcacactttttttgttag
gacgtataccaaggaattgtcgtttcaaacgacgtatagagacgttcatgtcatctatcttgttgataggtcttcatataaagtgcccgttcc
tgtagatgaagctggatatataaatttgaatggtcaatgtttttccgctgccgagattagaaaccagggaataaattatcgggtatatcata
aagatgataatacaaacaatacaatgagattatatcgtttgaaatttggatctactattaatactcgttacatatccacgcctgaatttcagttc
acatatggaacccattggttatataaaagctcatcttctattaattgcatagtaactgatactctggctaaatctgattacccatatgagaattt
tatactcggtacaggtgaatcggttgagatatcaccattttttaatggaacttctaaagaggtatttaatgaacagatgtactatttttcaatga
agaacaattacaccatgttagaaaaattagacgaacctaatggaccaaagaaaactatccctacgattgcttttctacaaaagggtgaca
cattgttttcatgggaggttaaggaacagactaattctcattgcaaatatacagcatggacaaaaaaacaccatgcattgcgagcagaca
tgacaaatagttatcattttatgatgaaggacactacagcaacaatggtaacaacaaagaacactataaatcttacgggcaccgaatacg
agtgtgtgaaaaatgatattgaaaaatatattacagatacgtttcagaacaaatataacaatacacacaacaaaacagaaaactatagtgt
ttatgaaacaactacaggtctaatattagtttggcagcctattgttaggaaatcaattaaagagttgaaagaatttatcaatgaaacccaac
atactcgttataagcgagaaattgatggaagtgactcacttgtttacgctagtttgcagtatatgtacgatgctttgagagagtatataaatg
ctggtttcgcccagatagctgaggcgtggtgcgaagatcagaaaagaaccaatgaagttctttccgaattagctaaaataagtccatcta
atgtcatgtctgtaatctacgataaatcattatccgcaaaattagttggagatgcaatttccgtatcttcgtgtgtcaatgtaaatcagtctac
ggtgaaggtgcataaagatatgagaatatacgccaatgggacggcgaatagggagacgtgtttttctcgcccggtcgttacgtttgaatt
ttcgaataataattcagttcaacaatttacgggtcaattggggccgaggaatgagattctgttaggcacgtatagagtggaaaaatgtgaa
cgtaacagtgttaaagttttttttgcgggtaaggaggcgtattttttttacaattacatttacacgaaaacggttaatatatctgatatcaatgtc
gtagatacttttatccaccttaatataaagccgttagagaatacagactttgaggtattgcgaatgtattctaaaaatgagttagcgcaggct
aacatatttgatctggaaagtcttttgagagatattaattcatataggagtgcgctttataatattgaatctaggatagctccaaggaaacca
gattacgtctcaggtgtggattcttttctgcatgctcttggtataggtgcacccggagggttgggtgccgctctgggtatggcgacgggg
gcggtcacagatttcctgactggcattttttctttctttaagaatccattcgggggacttttttccatgctgttttttgttttgcttgttttcttgat
attttccgtgtattacagacagaagaatatatatacaaacccggtaggtgcgttgtttccgtatgcaaatagttcttcgggaactgtaatatctaa
tacgcattcctattatgagacaaacaataaacaagagtttgagaatgacaggaaacctgatacgtccaatgccgtctctgagggcagtg
ccaataagtactctcaagaagatgccgtctgtatgttaatggctataaagaacctgggggatgcgtacagacggaagaatgccacaaa
gcctagcccgagcgtattagataagatacgtcacttggaatatcagcagctatccaccgaggacgtgtgacctgacgggcatgacatt
ctttaatccatatataaagaaaaaatataaccctgacgttcccgccgcctgtcaatacatccagattcttcctaaggggattatacatgatg
gcgagaagggattgcttaaaaacctgtgcgagaaagaaccgcggatgttttatagggagaaacagtacctgttggataaagacatgaa
atggccgtccctaaaaagaaaatgcagtgattctctatcatcatcgggggtgctgaaattccacgtgtacgacgccattgaatccgtgct
gtgtaccgaggacattgaatatcttccgtacaactacagacatcatgtaattcctacgggaactgtgatacggttgttcggtaaaactgta
aatggtgtaaaaatctgcgtcaatgtgtttggacagctatcatacatatactgtgaagtgcccagcgaaacatacctgcatgatgttatga
aaaaatacatgtatgagaataacaagaattttaccttttctaccgcggtggacaataaattttctatgtatggattcaataggtctccggtga
ctgtgtataagatcacttttgctgactactttgccgcgcgcgaggtcgggcgtatgctcgaagcgcaaaaaataaaagtgtatgagaca
agtgtggacgttctgtccagattctacatcgataatgactatcccagtttcggctggtacactgtacaaaaatattcaatcaatgaatacaa
attctcgaatcaaagtcttgagattagctgttccgttaaggatctctgcccactcgatgaggacatatggcccgaatatgactgcctgactt
ttgatatagaatgtatgagtgaatcggggggattcccaaatgcctcggtacagtcagacattgtcattcagatatcagccgtactgcaca
agacgggcagtgataatatggacgttcatctctttacattggggacttgcgaccccatagacgatgcccacatctatgagtttccctgtga
atacgaactcatctatgcttttctaatatttgtgaaacaggtatcgccagaaatcataacagggtataacataataaattttgatttgaactact
tgatcatcagagccacgaaattgtataggctgggtgtcggagaatttacaaaattgaaaaagggactattgtgtatcaagtcgactaatg
attttaaacaaggatttaactgtataaaaacaaaggtatacgcatccgggatgatatgcatagatttttaccctgtgtgtgtaggaaaaata
acgtccgataattataaactggacaccattgccaacctgtgtctgggaaaacacaaggaagatatgccctataagcgtatacctatcgaa
ttcatatctggttctggcggacgttgcaaagtgggcagatattgtatacaggattcgatgatagttaaggagttgttttgtaaattcaattacc
atatggaagcatctgctatttctaagttggcctacttgcccatcaggaaggtgataaatgatggacagcagagaagggtgttcacgtgta
tccttcatgagactagaaagagaaatatgctgatacccgacaagatagcgccgcgggagaccggcgaggacgagagctacagggg
cgccactgttctggacccacacgtgggttactatgcttctccagtgattgtcatggattttgccagtttgtaccccagcataatgatagcca
ataatctgtgttactctaccctgatcttaaatgacgaggacgtgacggggatcgacgagaaagatattctgacggtgcatgtaaacaaga
ataccgtgtacaggttcgttaggagcagcgtcagggagtctatactcggcacgctgctgtctagatggctcaggaagagaaaggaagt
gaaggcgcgcatgaaacgctgtgaggaccctatgttggcactgatacttgacaagcagcagcttgccctcaaggtgacgtgcaatgc
gttttacggcttcacgggagccgtgcacggtctgctgccgtgtctccctctagcggcgtccatcaccagcatagggcgggacatgctta
ggcagacgagtgactttatcaacaatgtcctttcgtctagagaatacgtgtcagagaagttcagtctctcagacggtgattttcaggggg
atttttccctgaatgtcatttacggagacacggatagcgtgttcgtgtgtgtcaagaacatcgtgcccgagaggctggccggcatatgtg
ctgacatagctgctcatgtgagctcgcagttgttcatcgagcctatcaaattagagtttgagaagatgttcctccccttgatgttgatatgca
agaagaggtatgtgggaaagatgtttgattcggacgagcttgtcatgaagggcgtagaactcgttagaaagacatcctgtccttttgtga
agaacattgtcaaggagatagtcagattgttgttctgggattccgaggttgccagggcggcggtcgagctctctcagatgagctgcgac
gaagtgatacggaacggattgcctgcggggatacacaagatcattgacatcataattggcgcgagggataggttgtacaccaataagg
tggatgtcaatgagcttgtcctgtccaccatgctgtctaaagagataagcatgtataagcagccaaacctgccgcacctccgtgtgatag
agagattgagaaatagaaacgaggaggtgcccgtggtcggggacagagtgtcatatgtgctgatctcatccatggataagaatgttgc
gaactatgagctctcggaggatcccctctacgtcatagagaacaatatacccataaacgccgaaagatactttgaacagctggtgaaag
cggtctctaacataatctgcccaataatccccaaggacactctcaaaaaagaacggtttttattgggggtgataccggcgcgcgtatatc
tacacagtagttttagggacagtgtcattgttatgtaaaaaaaagaaccattaaataaattttacttgaaacagcacacattgctgagggtg
tttctgtacttttcgtatttttcatttatgtcatccgggatgt
AF268039.2 Porcine cytomegalovirus strain (nucleotides 539-929)
glycoprotein B (gB)
SEQ ID NO: 5
atgacagtgagc agtcggaatt tattccggat tgttttattg attaactatg gatatctcgtgtcatctaat tctacgacat
ctacaacttc ggcatctgaa tcatcggtta ccaccggatctcccggtact gtcagttctg aaacagcgac accggaagtt
tctacggctt ttgtgaacattaccggaaat ttttcacagg aatacactga agcaagcgat gatgaaaaat
atccttttcgtgtatgcaat atggccgttg gaacagatct atatagattt gataattata tcacttgtaataagtataat acggagactc
agtattctga aggtattttg ttattgttta agaaaaatat agttccgcac actttttttg ttaggacgta taccaaggaa
AF268039.2 Porcine cytomegalovirus strain (nucleotides 2771-3118);
glycoprotein B (gB)
SEQ ID NO: 6
ttgatatttt ccgtgtatta cagacagaag aatatatata caaacccggtaggtgcgttg tttccgtatg caaatagttc
ttcgggaact gtaatatcta atacgcattcctattatgag acaaacaata aacaagagtt tgagaatgac aggaaacctg
atacgtccaatgccgtctct gagggcagtg ccaataagta ctctcaagaa gatgccgtct gtatgttaatggctataaag
aacctggggg atgcgtacag acggaagaat gccacaaagc ctagcccgagcgtattagat aagatacgtc acttggaata
tcagcagcta tccaccgagg acgtgtga
AF268039.2 Porcine cytomegalovirus strain (nucleotides 539-3118);
glycoprotein B (gB)
SEQ ID NO: 7
atgacagtgagc agtcggaatt tattccggat tgttttattg attaactatg gatatctcgtgtcatctaat tctacgacat
ctacaacttc ggcatctgaa tcatcggtta ccaccggatctcccggtact gtcagttctg aaacagcgac accggaagtt
tctacggctt ttgtgaacattaccggaaat ttttcacagg aatacactga agcaagcgat gatgaaaaat
atccttttcgtgtatgcaat atggccgttg gaacagatct atatagattt gataattata tcacttgtaataagtataat acggagactc
agtattctga aggtattttg ttattgttta agaaaaatatagttccgcac actttttttg ttaggacgta taccaaggaa ttgtcgtttc
aaacgacgta tagagacgtt catgtcatct atcttgttga taggtcttca tataaagtgc ccgttcctgtagatgaagct
ggatatataa atttgaatgg tcaatgtttt tccgctgccg agattagaaa ccagggaata aattatcggg tatatcataa
agatgataat acaaacaata caatgagatt atatcgtttg aaatttggat ctactattaa tactcgttac atatccacgc
ctgaatttcagttcacatat ggaacccatt ggttatataa aagctcatct tctattaatt gcatagtaactgatactctg gctaaatctg
attacccata tgagaatttt atactcggta caggtgaatcggttgagata tcaccatttt ttaatggaac ttctaaagag gtatttaatg
aacagatgtactatttttca atgaagaaca attacaccat gttagaaaaa ttagacgaac ctaatggaccaaagaaaact
atccctacga ttgcttttct acaaaagggt gacacattgt tttcatgggaggttaaggaa cagactaatt ctcattgcaa
atatacagca tggacaaaaa aacaccatgcattgcgagca gacatgacaa atagttatca ttttatgatg aaggacacta
cagcaacaatggtaacaaca aagaacacta taaatcttac gggcaccgaa tacgagtgtg tgaaaaatgatattgaaaaa
tatattacag atacgtttca gaacaaatat aacaatacac acaacaaaacagaaaactat agtgtttatg aaacaactac
aggtctaata ttagtttggc agcctattgttaggaaatca attaaagagt tgaaagaatt tatcaatgaa acccaacata
ctcgttataagcgagaaatt gatggaagtg actcacttgt ttacgctagt ttgcagtata tgtacgatgctttgagagag
tatataaatg ctggtttcgc ccagatagct gaggcgtggt gcgaagatcagaaaagaacc aatgaagttc tttccgaatt
agctaaaata agtccatcta atgtcatgtctgtaatctac gataaatcat tatccgcaaa attagttgga gatgcaattt
ccgtatcttcgtgtgtcaat gtaaatcagt ctacggtgaa ggtgcataaa gatatgagaa tatacgccaatgggacggcg
aatagggaga cgtgtttttc tcgcccggtc gttacgtttg aattttcgaa taataattca gttcaacaat ttacgggtca
attggggccg aggaatgaga ttctgttaggcacgtataga gtggaaaaat gtgaacgtaa cagtgttaaa gttttttttg
cgggtaaggaggcgtatttt ttttacaatt acatttacac gaaaacggtt aatatatctg atatcaatgtcgtagatact tttatccacc
ttaatataaa gccgttagag aatacagact ttgaggtattgcgaatgtat tctaaaaatg agttagcgca ggctaacata
tttgatctgg aaagtctttt gagagatatt aattcatata ggagtgcgct ttataatatt gaatctagga
tagctccaaggaaaccagat tacgtctcag gtgtggattc ttttctgcat gctcttggta taggtgcacc cggagggttg
ggtgccgctc tgggtatggc gacgggggcg gtcacagatt tcctgactggcattttttct ttctttaaga atccattcgg
gggacttttt tccatgctgt tttttgttttgcttgttttc ttgatatttt ccgtgtatta cagacagaag aatatatata
caaacccggtaggtgcgttg tttccgtatg caaatagttc ttcgggaact gtaatatcta atacgcattcctattatgag
acaaacaata aacaagagtt tgagaatgac aggaaacctg atacgtccaatgccgtctct gagggcagtg ccaataagta
ctctcaagaa gatgccgtct gtatgttaatggctataaag aacctggggg atgcgtacag acggaagaat gccacaaagc
ctagcccgag cgtattagat aagatacgtc acttggaata tcagcagcta tccaccgagg acgtgtga
AF268039.2 Porcine cytomegalovirus strain (nucleotides 2309-2771)
SEQ ID NO: 10
aa cagtgttaaa gttttttttg cgggtaagga ggcgtatttt ttttacaatt acatttacac gaaaacggtt aatatatctg
atatcaatgt cgtagatact tttatccacc ttaatataaa gccgttagag aatacagact ttgaggtatt gcgaatgtat
tctaaaaatg agttagcgca ggctaacata tttgatctgg aaagtctttt gagagatatt aattcatata ggagtgcgct
ttataatatt gaatctagga tagctccaag gaaaccagat tacgtctcag gtgtggattc ttttctgcat gctcttggta
taggtgcacccggagggttg ggtgccgctc tgggtatggc gacgggggcg gtcacagatt tcctgactgg cattttttct
ttctttaaga atccattcgg gggacttttt tccatgctgt tttttgtttt gcttgttttc t

In some embodiments of the methods disclosed herein, the one or more engineered PCMV antigens comprise glycoprotein gH and/or glycoprotein gL. In some embodiments, the one or more engineered PCMV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 18 or 22. The one or more engineered PCMV antigens can comprise the amino acid sequence of SEQ ID NO: 18 and/or 22. The engineered PCMV antigens can comprise glycoprotein gL or SEQ ID NO: 22.

The engineered PCMV antigens can comprise glycoprotein gH or an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 22.

AGT99272.1 glycoprotein L (U82) [Suid betaherpesvirus 2]
SEQ ID NO: 22
MTEQCVQITRNCSGELDLNELDINEGTENHSLVDPKKISTIIKYEPPKYLFNIKLLKNSS
FLNDSLLLRNNVQQVRTLLTLMKTDADTWSQVFRGYELCHGRDVLHTCVGENNTC
ATYNLRTLSYNPSPFTENVVGFELQKTSMNLNLTILIMLRNDFNNQSKVVRLPITSVA
FFDALFNIIEFYRLNTESNINILTELAHYGYTFPELQVHRPILIVREHYKKHEIRNGTHY
NTTYLNVTTQ

The one or more engineered PCMV antigens can comprise glycoprotein gH or SEQ ID NO: 18. The one or more engineered PCMV antigens can comprise glycoprotein gH or an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 18.

AGT99240.1 glycoprotein H (U48) [Suid betaherpesvirus 2]
SEQ ID NO: 18
MELKHPFYFLQILNLVRAWNDIEIVNRSLTCKSESSNHTLVHPGLITFNFYSGEDSVRT
YRMPKCLFTTEIAERIFASVNLSETLEDYKSRFKKQFLPPVHANYKVIINQNTPTNTNP
VSETNNHNYDNNYITLKDFQWIPIEQDCLLYNVSTVIFPIVIQCRNLTFKVKDISITFSF
TNHFFVFSITIGNSKPIDIVFGNIERINVKSPYRKVDLLYRQTVTDDMLVLATVNDTSN
DYSSHFGKNIFDEILQTNYVNVETAIGFTSRFIANYFKNDLCQNRKEDFYKLIFLYGLS
SFAYNQVNDSRKILLPKVLQLHTDMATLTLFLRICIGEEIEFVHKLKKDNVTYEIIKDG
NRYKNIPSDPTSLGFVSYMSQQNVSDDNLSEIEKRALTLLDMYRRTTNITREDRKTIT
MLQLIARKSHNMTTRRTLLNVLTTMCTPTEIQKWAENIQAHPDLSGLYSPCAFSGRF
DLTPGYLERLVRSTGRDTSIAKRMHNLILKNKPKTLEAFNLSCVSTERDLIVITLVNVS
YIISDRIMVKGLSYIITATIINTDIIITAVFSNETCTAIQYKHTEGTILTMANNTNGEHLL
NTVLLQYDSVEGVVRLVYIDTKEELLRILDPNNDIIVRSPRTHYLLLLNNGTIFNVSPV
HVFFSETSIILLFVYFIIALLLIFVLYIMCRVF

In some embodiments, the one or more engineered PCMV antigens can comprise the amino acid sequence of SEQ ID NO: 18 and/or 22 and a tag. The tag can comprise the amino acid sequence of SEQ ID NO: 33 HHHHHHHHHH. The tag can also be a 10×his tag (SEQ ID NO: 33). The tag can be at the N-terminus or C-terminus of the engineered PCMV antigen.

In some embodiments, the one or more engineered PCMV antigens described herein are encoded by a polynucleotide sequence selected from SEQ ID NO: 17, 21, or a combination thereof. The glycoprotein gH can be encoded by a polynucleotide sequence of SEQ ID NO: 17.

KF017583.1: c64486-62438 Porcine cytomegalovirus strain BJ09 (U48), complete genome
SEQ ID NO: 17
atggaactcaaacaccccttttactttctgcagattctgaacctcgtaagagcgtggaatgatatcgaaatcgtcaacaggtctctgacgt
gcaaatcagagtcgtctaaccacactctagtacacccggggctgatcacctttaatttctactccggcgaggacagcgtcaggacatac
agaatgccaaaatgtctgttcaccacggagatcgccgaaaggatcttcgcatccgtcaacctatcagaaacgctcgaggactacaaat
cacgcttcaagaaacaattcttacctcccgtacacgcaaattataaggtgataatcaatcaaaatacgcccacaaatactaaccccgtatc
agaaaccaataaccataattacgataataactatataaccctcaaggatttccaatggatacccatagaacaggactgtctgttgtataac
gtaagcactgttatttttccgatcgtcatacaatgtaggaatcttaccttcaaggtgaaagatatatcaataacattttcattcaccaatcat
ttttttgtgttttcaataactatcggtaactctaaacccatcgatatagtcttcggaaacatagaacggatcaatgttaagagtccatatagg
aaagtagatctattgtatagacagaccgtcaccgatgatatgctcgtactggcaacggtaaacgatacatccaacgactactcgtcgcatttt
gggaaaaatatatttgatgaaatactccagacaaattacgttaacgtggagaccgcgatcggcttcacctcgcgattcatcgcgaattactt
taaaaatgacctgtgccaaaatcgcaaggaagacttttacaagttgatatttctttacggactcagttctttcgcatacaaccaggtgaacg
attccaggaagatattactgcctaaggtacttcagcttcacactgatatggccacgctaacattatttttaaggatatgcatcggggagga
gatagaattcgtacacaaactaaaaaaagataacgtcacgtacgaaataataaaagatggaaataggtacaaaaatatcccgtcagatc
ccacatcacttggattcgtttcatatatgagccagcagaacgtatccgacgataacctgtccgaaatcgaaaaaagagccctaacactct
tagatatgtacagaagaaccacaaacataacacgcgaggacaggaaaaccataaccatgctgcagctcatcgccaggaaaagtcac
aacatgactaccaggaggacccttttgaacgtgttgactaccatgtgtacccccacggaaatccagaaatgggggaaaatatccagg
cccaccccgacctatccggtctctattcaccgtgtgctttcagtggacgattcgatctgacacccggttacctggagagactcgtgcgga
gtaccggtagagacacgtccatagctaaacgcatgcacaacctcattctaaaaaacaaaccaaaaacattggaggcctttaatctgagc
tgcgtgtccaccgaacgcgacctcatagtgataacccttgtaaatgtctcttatataatatcggatcgcataatggtgaaggggttgtcgta
cataattacagcaactataataaatacagatatcatcataacggcggtgttctcgaacgagacctgtacggcgattcaatataaacacac
agaaggaaccatcctgacgatggccaacaataccaacggcgaacatttattaaacaccgttctccttcagtacgattctgtggaagggg
tcgtacggctcgtgtacatagatacaaaagaagagctgttacgtatcctcgatcccaataacgatataatcgtacgaagcccgaggact
cattatcttttattgctgaacaacgggacgatattcaacgtatcgcccgtacacgtcttcttcagcgaaacatccatcatcctgcttttcgta
tatttcatcatcgccttgttacttatattcgttttgtatatcatgtgccgcgtattttaa
The glycoprotein gL can be encoded by a polynucleotide sequence of SEQ ID NO: 21.
KF017583.1: c106307-105552 Porcine cytomegalovirus strain BJ09 (U82), complete genome
SEQ ID NO: 21
atggcgttgaaagactggctacgatcgaacataaaaacggaacacgatgaattgacaaacgaacagatcagcacacttttcgggatat
ctaggagctggattgattttattgatctatcatctatagatgtccactcattgagaaaaacgtactctattgtacgtgaactacggaataata
atattgtgtttcctgaacacgactccattcacgtatggagtcacctgtgtacacctgacagagtgaaggttgttattctcggacaagatccct
atccagacgagagaggttgcggtctcgcatttagcacaaggagagaccaacccgttccagaatctctgcgtaatatatacaaagaact
ggccaggagttcattttttcgaataccgcagagcggttgcttggagaaatggtgtgatgaaggggtcttgctgctaaatacagttttcacc
gtaaccgcggggaagccatcctcccactccaacctcggctggcaggtattgtccgagaaggtcatcacatccatatcatgtaacctga
aaaacgtggtgttcctactgtggggaggacatgcgaaaaaatttatttccctgatagacgattcaaagcacctgatcctaacgtccagtc
atccctcgcctaaggtgacgtcttcaaaagacccttttctcggaaacaatcactttatcaggaccaaccagtacttgcagagtcatggga
agacgcccgttaattggaacattttaacagactaa

In some embodiments of the methods disclosed herein, the one or more engineered PCMV antigens comprise U100p (gQ). In that embodiment, the one or more engineered PCMV antigens can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 24. Alternatively, the one or more engineered PCMV antigens can comprise the amino acid sequence of SEQ ID NO: 24.

AGT99280.1 putative protein U100p (gQ) [Suid betaherpesvirus 2]
SEQ ID NO: 24
MFKPARARAGGYNHAEIFLQSFQSNGSDLKYLYPFSLSQFLARPTAVPQLPHTPCNG
TEQSGTSTGMFYYGEDKICVDIRNTDLSMYRIQAAVVTIPPMLASAVDMHYTLLHNH
TSDNSTAQRFVSGLPIVGLQSLASVLDEEMHVILEKNYTVQGNSW

In some embodiments, the one or more engineered PCMV antigens can comprise glycoprotein gN and/or glycoprotein gM.

In that embodiment, the glycoprotein gN can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 16; or the amino acid sequence of SEQ ID NO:16 or 20. Alternatively, the glycoprotein gN can be encoded by a polynucleotide sequence of SEQ ID NO: 15.

In some embodiments, the glycoprotein gM can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 20; or can comprise the amino acid sequence of SEQ ID NO: 20. Alternatively, the glycoprotein gM can be encoded by a polynucleotide sequence selected to SEQ ID NO: 19.

KF017583.1: 61918-62247 Porcine cytomegalovirus strain BJ09 (U46), complete genome
SEQ ID NO: 15
atggctatattattaaagatagacctacagtatgagcgtcaccgtacctttttatgtcatataattatgacctggatcgtggttttgtgtttt
gctttatttaacgtcatcgtggcgagtgagggattatcttttccctctcataagatcgatcataatacggaattttataaacagtcgtgccag
tcccacgtattcgaggttgagttcacctcgttcaccacgatatggctactcataaatagccttttcctgttatcatctgttggaatttttttg
aaattctggtgttacaaatcctttgcggaagagacggttaagggatactga
AGT99239.1 glycoprotein N (U46) [Suid betaherpesvirus 2]
SEQ ID NO: 16
MAILLKIDLQYERHRTFLCHIIMTWIVVLCFALFNVIVASEGLSFPSHKIDHNTEFYKQ
SCQSHVFEVEFTSFTTIWLLINSLFLLSSVGIFLKFWCYKSFAEETVKGY
KF017583.1: c92555-91524 Porcine cytomegalovirus strain BJ09 (U72; glycoprotein M),
complete genome
SEQ ID NO: 19
atgtccctaagcagggtggacagcattaatatcaaattatgggtgtctgcgatagtctgcgtctgtctgacttatctaaacgtcataatatac
ctgatatcctcgcacgtgcccggactcggcttcccctgcgcatattttgaaatcgttgacctctcctctataaatatgacgacatacaacga
tatgcatacgctgacaccgcagttgtttatgacccctcccatgacagtgacctacgtagttttcacaatgatagtctacaccattatcacag
cgtactacgtggcgtgctgcgtatccgtgtgtctgtactcgtccaaaaacacaagcggactgaatcactccaccaaacacataaaatgc
ctcggggacattttctcctgtctgcttttcatcctgtgtatggacacgtttcagcttttcatcaccacgctatcgttccggttgcccgcgata
gccgcttttactttttgcatacacttcctgtgctctacgttctacacgggcactctgctcacacagtacgccagctacgagaaactaggcgc
cctctttaagcagaacaaatttctgtcgatgatggtcacggcgaagagtgtggtcgtgagcaccctcggtgtaaacctgggtatatgttg
catgatattctccctcacgctcttcctgggtgtggggaacagtttctacattaaaacggccaacataacattcgcatcaataaacctgttca
tgattttaatgatattttattgcatactcatagaatcggtgttacacagagcaatgaagatgaattttggctttcactgtggtctatttatag
gactatgtggattattatatcccacgcacaaatatgaaagtatctatgcatcggagtggagctacaccatagatgtcaatctaggtatcgttg
gaatcgtttggattatcttcacgatctgtagaatcataagaacgctcttgccactcagacacagaaagcattataagccactcctagacgaag
agactgaacctttaaaataa
AGT99264.1 glycoprotein M (U72) [Suid betaherpesvirus 2]
SEQ ID NO: 20
MSLSRVDSINIKLWVSAIVCVCLTYLNVIIYLISSHVPGLGFPCAYFEIVDLSSINMTTY
NDMHTLTPQLFMTPPMTVTYVVFTMIVYTIITAYYVACCVSVCLYSSKNTSGLNHST
KHIKCLGDIFSCLLFILCMDTFQLFITTLSFRLPAIAAFTFCIHFLCSTFYTGTLLTQYAS
YEKLGALFKQNKFLSMMVTAKSVVVSTLGVNLGICCMIFSLTLFLGVGNSFYIKTANI
TFASINLFMILMIFYCILIESVLHRAMKMNFGFHCGLFIGLCGLLYPTHKYESIYASEW
SYTIDVNLGIVGIVWIIFTICRIIRTLLPLRHRKHYKPLLDEETEPLK

The one or more engineered PCMV antigens can also comprise one or more major tegument phosphoproteins (pp65). In that embodiment, the pp65 can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 12 or 14; or can comprise the amino acid sequence of SEQ ID NO: 12 or 14. Alternatively, the pp65 can be encoded by a polynucleotide sequence selected to SEQ ID NO: 11 or 13.

KF017583.1: c72304-70307 Porcine cytomegalovirus strain BJ09 (major tegument phosphoprotein
pp65; U54A), complete genome
SEQ ID NO: 11
atgagccgcatgtatccaaaatggacgtcctacatcacacaaataaaaataaacaatgatgttttgatatcgccgctagagtgcaaatgc
ctcctactcagcctaaaaatagaagtaccaataggatcatgtgttttgatgtgccaagataatgaaacctccgtatatgttccgttctcgttc
ctggacgtatccaacgacggtaagattaaagtaaacctaataaataaaactaacaattacatcagactaaaaagaacgcccattgtgctc
aatgtcttcgccatatcatgcaacgtccccgtcatcatcaccacaccggtgaaggaactaaaggaattagtcataacaaatcacatggta
tcgagcacaaagaatgaaacactaaccacctacaaggctggatcaaagatatgcttctgcatcaaaaccaattggaaaacaaaactga
aaagctctacgtttacaagattcgtgtcaagcgcatggacatttctgtacagcttctacaaggcgcatccgttcaattgcgcattcatcgaa
cacatatctctaaacggcggctatataaaaaatatatgtatatatgacaacagcgtgttgttatttgatatatgtatcgacaaaaacataaac
gatccggagccgctaccgtcggacgtattcataagtattctttttcagaaccatgacgcagatctagttacgaacgataactgtgatccgt
acataactaaaggagctcatccggcagaactctccgtctatgccaacactgacatgttgatcaaacccaaacagcaaacatcgatcact
tatgatgtaaagtatacgtcgtcttcactaaacggagtatttctacctctgttttttaacggtattgaaatctacccagtcgtgtggaatagcg
agtctaatctgtctctgccgttacgcggaggggccaatacagttcacatacggagaggacagttattagggaagatttatttattttcaaat
gacttattcaaaacaagtgatatcacatcacaccctagcctcgaagaatcctcatccataatccttagtgaccatgtccctgaatcgagcg
gtcttggagacaggtcaagaccactactgtccctcagaagtataccacagacaagcacatcatccagcgtctcagaaacactgtccgt
ctctacgaaacctaagagggcgcagaaaaaaaccagttcttcggatagcacatatttttccacgccgtcaaagaaacctcataaaaaaa
aagcagaccctttcggcacaccatatgcaaaacctgtcagcgaaaaggggtcacccaagaaacttcgaatatcctctggcgatacaca
ccgaaaaacagagccaccccccaaatccacacctatatcgattcaaaaactcaagatatcagacaccagcgacgacgaatcagaaat
agaagatataaacgcacaaatcttttttatcactccccccgacgaagaacaggctaaaggtcccctgtcaacgcggacatcatctccgc
gtatagacgatactcccatgggcacccctgtcgattttggattcgccgaatccgacgcggaaagagaaaagacacccatacccagtcc
cattatatttggagatgtcatagtgataagtagcagcgacgacgataatgaaaaaccgcacaagactagacccaggctcgtcaaaccc
agaccactggggaaatattccaagccgctcagctcggacgaggatgtgaacatagggctggctcagcaagccgtctccgccataaa
cgtacacggcgaatacactctgaggtgcaaacagttcctaaacgcctcagaaccgatctgcatcagacttcacagtctaggtctcacgc
ttcccatacagaacatgtgccccatgaaacaatgcatgctcacacaaacaaatgtgtattttgatatatctggcccagcgccgaaatacat
cacaacactagaaataaaataa
AGT99246.1 major tegument phosphoprotein pp65; U54A [Suid betaherpesvirus 2]
SEQ ID NO: 12
MSRMYPKWTSYITQIKINNDVLISPLECKCLLLSLKIEVPIGSCVLMCQDNETSVYVPF
SFLDVSNDGKIKVNLINKTNNYIRLKRTPIVLNVFAISCNVPVIITTPVKELKELVITNH
MVSSTKNETLTTYKAGSKICFCIKTNWKTKLKSSTFTRFVSSAWTFLYSFYKAHPFNC
AFIEHISLNGGYIKNICIYDNSVLLFDICIDKNINDPEPLPSDVFISILFQNHDADLVTND
NCDPYITKGAHPAELSVYANTDMLIKPKQQTSITYDVKYTSSSLNGVFLPLFFNGIEIY
PVVWNSESNLSLPLRGGANTVHIRRGQLLGKIYLFSNDLFKTSDITSHPSLEESSSIILS
DHVPESSGLGDRSRPLLSLRSIPQTSTSSSVSETLSVSTKPKRAQKKTSSSDSTYFSTPS
KKPHKKKADPFGTPYAKPVSEKGSPKKLRISSGDTHRKTEPPPKSTPISIQKLKISDTS
DDESEIEDINAQIFFITPPDEEQAKGPLSTRTSSPRIDDTPMGTPVDFGFAESDAEREKT
PIPSPIIFGDVIVISSSDDDNEKPHKTRPRLVKPRPLGKYSKPLSSDEDVNIGLAQQAVS
AINVHGEYTLRCKQFLNASEPICIRLHSLGLTLPIQNMCPMKQCMLTQTNVYFDISGP
APKYITTLEIK
KF017583.1: c73541-72354 Porcine cytomegalovirus strain BJ09 (U54B), complete genome
SEQ ID NO: 13
atggcaaagcacaccgaaacaaacctgctgtctttaattgtctcgcccgacaaagcatacagtcctagcgaaagatcaaaa
gtgttatccctggggcatagtattcacacacgaaccgatgccgtcatactagccggagcagacaccttcacacctttcggggtgacgct
ttccgcatcatacgggaggctaggagcaccgatatttatcgtttctaatccaggtgccagaactagagaacccatacgcctcaacctaat
agccataccgatcgtagaagtgactgataaatttttggtgtataccacactcaccgaagacaaggaaaataatagaaaggctccgagaa
gagccaaagccagccgcgtcgacgtatctcacgtcgacaccgaatcgactcgcacgctcaccatttccgtctacggactgcattggag
catcataacaaccccgagacagcacgaactcggagcaaatctatattttcagagtatatgggattgcgcatattacccagatgcattcag
acccatgtatagcacacaccctgggatctttgttagacaagtgagagtgacaggtgacgatcagtacatggtatccctgaggtaccacc
cgacagaatctcccccgagccctccaaaagacctgatggtaaagatagaactacacatcaactccctagactccgaagcggtcatcct
atacaaacgggccccgtctatggtaccggtcccgggcagaaactgcgtagatgtgctgtgtccggaggacgtcagactgatacccgg
ggaggtaaaacatgtaaggatatcgaatcaatacatcactaaccaccacaccgggctattcatccccgaggtgttcgaggacgtcaca
gtatatccaaaagtatggaatgagggggaatcgctaatcgcatcatttctatcgaacaaggactccgtaagcgtagtgggtgaaggata
caagatcggctgcgtctacttcctaaaaaacccctttgtaaatagacagcacggaaacgacgaaccgtcccagagcatcacattgaaat
tagacacggccaataagacagtttcgtttctgggtatcgaaataccgttagacgcactggaaaccctcgtcatggatcaaaccgcgaca
tccacggcagaagtaacattacattgcgaaggacgcgtataa
AGT99247.1 major tegument phosphoprotein pp65; U54B [Suid betaherpesvirus 2]
SEQ ID NO: 14
MAKHTETNLLSLIVSPDKAYSPSERSKVLSLGHSIHTRTDAVILAGADTFTPFGVTLSA
SYGRLGAPIFIVSNPGARTREPIRLNLIAIPIVEVTDKFLVYTTLTEDKENNRKAPRRAK
ASRVDVSHVDTESTRTLTISVYGLHWSIITTPRQHELGANLYFQSIWDCAYYPDAFRP
MYSTHPGIFVRQVRVTGDDQYMVSLRYHPTESPPSPPKDLMVKIELHINSLDSEAVIL
YKRAPSMVPVPGRNCVDVLCPEDVRLIPGEVKHVRISNQYITNHHTGLFIPEVFEDVT
VYPKVWNEGESLIASFLSNKDSVSVVGEGYKIGCVYFLKNPFVNRQHGNDEPSQSIT
LKLDTANKTVSFLGIEIPLDALETLVMDQTATSTAEVTLHCEGRV

On aspect of the present disclosure provides an automated serology method for detecting an anti-porcine cytomegalovirus (PCMV) antibody in a porcine animal, the method comprising: contacting one or more engineered PCMV antigens comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 12, 14, 16, 18, 20, 22, 24, 25, 26, 27, 28, 29, 30, 31, and 32 with a biological sample from the porcine animal; incubating the one or more engineered PCMV antigens with the biological sample for about 10 minutes to about 120 minutes; and detecting the binding of the one or more engineered PCMV antigens to the biological sample by immunodetection and/or chemiluminescent detection.

The method disclosed herein can be a multiplex antibody detection assay. For example, the method disclosed herein may be used for the detection of multiple antigens in a sample. The presence of antibodies to multiple agents/or antigens can be attributed to concurrent infections as well as asymptomatic, or past infections. This is a great improvement over traditional standard immunoassays, such as fluorescent bead technology, enzyme-linked immunosorbent assays (ELISA), western blots, radioimmunoassays (RIA), and indirect immunofluorescence assays (IFA) that can only detect a single agent in a sample.

Indeed, ELISA, western blot, and IFA require measurement of each antibody separately, and thus are cumbersome and time consuming. Moreover, traditional ELISA, western blot, and IFA cannot be used for a high throughput analysis of multiple antibodies in a single sample of biological fluid. Such an analysis requires a method that can provide a parallel and a rapid analysis of a single sample comprising one or more antibodies against one or more pathogen. While ELISA only provides protein binding information, CE western techniques provide additional information about the target protein, such as molecular weight estimation (e.g., as size, size distributions) and protein intactness (e.g., isomers, fragments, and aggregates). As such, an automated capillary electrophoresis (CE) western can be more sensitive than an enzyme-linked immunosorbent assay (ELISA) for the quantitative analysis of targets proteins in a serum sample from immunized animals. CE-western techniques can be orthogonal methods of analyzing protein in a biological sample. They can complement traditional fluorescent detection and/or chemiluminescent detection methods such as an ELISA method.

The identification of pathogenically relevant antibodies in a bodily fluid, such as whole blood or serum can also be impeded by the presence of large amounts of natural antibodies in the sample. These natural antibodies can manifest themselves in complex staining patterns. Avrameas S., Immunol. Today 12 (5): 154-9 (1991). The presence of these natural antibodies can also complicate the differentiation of disease-associated antibodies from the complex background of “auto-immune noise.” Furthermore, as noted above, certain viruses can remain in a latent state and can therefore be undetectable using routine PCR-based assays.

Accordingly, the method described herein provides a system where more than one antibody can be detected using a single assay. Thus, in some embodiments, the one or more engineered PCMV antigens can comprise glycoprotein B (gB). The one or more engineered PCMV antigens can comprise gB, gH, and gL. In some embodiments, the one or more engineered PCMV antigens comprise gB, gM, and gN. In some embodiments, the one or more engineered PCMV antigens comprise gB, and a phosphoprotein pp65. In some embodiments, the one or more engineered PCMV antigens comprise gB, gH, gL, gM, gN, and U100(Q1). In some embodiments, the one or more engineered PCMV antigens comprise gB, gH, gL, gM, gN, U100(Q1), and a phosphoproteins pp65. In some embodiments, the one or more engineered PCMV antigens can comprise SEQ ID NO: 2, 3, 8, 9, 12, 14, 16, 18, 20, 22, 24, 25, 26, 27, 28, 29, 30, 31, and 32.

In some embodiments, the one or more engineered PCMV antigens can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 12, 14, 16, 18, 20, 22, 24, 25, 26, 27, 28, 29, 30, 31, 32, and any combination thereof.

### Tags

In some embodiments of the methods disclosed herein, the one or more engineered PCMV antigens can be conjugated to a tag protein. The tag protein can be selected from histidine tag (his-tag), polyhistidine (poly(His)) tag, small ubiquitin-like modifier tag (SUMO), a VariFlex C-Terminal solubility enhancement tag, a short peptide C-terminal tag, Thioredoxin (Trx) tag, a VariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Immunoglobulin Fc-tag, IgG-Fc-tag, Glutathione-S-transferase (GST) tag, maltose-binding protein tag (MBP), FLAG tag peptide (FLAG), streptavidin binding peptide tag (Strep-II; strep), calmodulin-binding protein tag (CBP), mutated dehalogenase tag (HaloTag), biotin, or staphylococcal Protein A (Protein A).

In some embodiments, the one or more engineered PCMV antigens can be conjugated to a poly histidine (poly-his) tag. The poly-histidine can be selected from 2×his-tag, 3×his-tag, 4×his-tag (SEQ ID NO: 44), 5×his-tag (SEQ ID NO: 45), 6×his-tag (SEQ ID NO: 43), 7×his-tag (SEQ ID NO: 46), 8×his-tag (SEQ ID NO: 47), 9×his-tag (SEQ ID NO: 48), or 10×his-tag (SEQ ID NO: 33). The poly-his tag can be a 10×his-tag (SEQ ID NO: 33) or can comprise the amino acid sequence of SEQ ID NO: 33. In some embodiments, the tagged engineered PCMV antigen is the PCMV envelope glycoprotein B (gB). The tagged engineered PCMV antigen can comprise the amino acid sequence of SEQ ID NO: 32. In some embodiments, the tagged engineered PCMV antigen can comprise the amino acid sequence of SEQ ID NO: 33 and an amino acid sequence selected from SEQ ID NO: 2, 3, 8, 9, 12, 14, 16, 18, 20, 22, 24, 25, 26, 27, 28, 29, 30, or 31. In that embodiment, SEQ ID NO: 33 can be at the N-terminus or C-terminus of SEQ ID NO: 2, 3, 8, 9, 12, 14, 16, 18, 20, 22, 24, 25, 26, 27, 28, 29, 30, or 31.

In some embodiments, the tagged engineered PCMV antigen can comprise the amino acid sequence of SEQ ID NO: 33 and an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 12, 14, 16, 18, 20, 22, 24, 25, 26, 27, 28, 29, 30, and 31. In that embodiment, SEQ ID NO: 33 can be at the N-terminus or C-terminus of an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 12, 14, 16, 18, 20, 22, 24, 25, 26, 27, 28, 29, 30, and 31.

A rapid high-throughput PCMV serology western blot technique using an automated capillary electrophoresis (CE) western (e.g., Simple Wes™) to detect PCMV anti-IgG antibodies in a sample from a subject was developed. The novel automated serology assays described herein include the use of porcine serum or a serum from a subject transplanted with a porcine cell, tissue or organ, the application of specific dilutions and antigen concentrations, and unique modifications to the Wes™ protocols. Together these modifications (e.g., optimizations) showed a consistent and reliable detection of anti-PCMV antibodies (e.g., anti-IgG antibodies) in tested samples.

### IV. PLHV Serology Assay

Another aspect of the present disclosure provides a method for detecting a porcine lymphotropic herpesvirus (PLHV) in a subject using the novel serology assays disclosed herein. In some embodiments, the porcine lymphotropic herpesvirus (PLHV) is selected from PLHV-1, PLHV-2, PLHV-3, or a combination thereof.

In some embodiments, the one or more engineered PLHV antigens are selected from the group consisting of envelope glycoprotein B (gB), envelope glycoprotein H (gH), envelope glycoprotein L (gL), envelope glycoprotein M (gM), envelope glycoprotein N (gN), major tegument phosphoprotein1 (U54A), major tegument phosphoprotein2 (U54B); and U100p.

The one or more engineered PLHV antigens can comprise glycoprotein B (gB). The PLHV glycoprotein B (gB) can comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 34, 35, 36, 37, 38, 39, 40, 41, 42, or a combination thereof. In some embodiments, the PLHV glycoprotein B (gB) comprises the amino acid sequence of SEQ ID NO: 34, 35, 36, 37, 38, 39, 40, 41, 42, or a combination thereof.

For example, the one or more engineered PLHV antigens can be PLHV-1 antigens comprising the amino acid sequence of SEQ ID NO: 34, 35, 36, or a combination thereof.

AM003269.1 glycoprotein envelope protein (complete sequence)
Porcine lymphotrophic herpesvirus 1
SEQ ID NO: 34
MLFYIHIYKCTMAGSLKLRGSVLALWYLYQVALYSLSIAETGVTSPPNTATWSTESP
LTGHYGTHDSSHGERGNNENRDSEEQNKNIYGSPSTFPYRVCSASGVGDVFRFQTDH
VCPDASDMVHSEGILLIYKQNIIPFMFRVRKYRKVVTTSTVYNGIYSDSITNQHTFYK
SIEPWETEKMDTIYQCFNSLRLNTGGNLLTYVDRDDINMTVFLQPVDGVTPDVKRYG
SQPELYLEPGWFWGSYRRRTTVNCELMDMFARSNPPFDFFVTATGDTVEMSPFWSG
EDDHENKMHEKPWFVSVINNYKVVDYQNRGTVPLGKTRIFLDREEYTLSWEKHLK
NMSYCPLTLWKAFYNGIQTDDSGSYHFVANDITASFTTSKEDMKEFNTTYHCLNEEI
KAEIEKKYAKVNSTHSKYGDLKYFKTDGGLYLVWQPLIQNRLLDAKNKLNNETYSR
RSRRQAESTTDPMMEMTGNGAGGEYSSENSITVAQVQYAYDNLRIRINNILEDLSKA
WCREQHRAALVWNELSKINPTSVMSMIYNRPVSAKRIGDVISVSNCIVVDQTSVSLH
KSLRLLSASDEKCFSRPPVTFKFMNDSTIYKGQLGVNNEILLTTTYLETCQENTEYYF
QAKTDMYIYKNYEHLKTVPLSSITTLDTFIALNFTLLENVDFKVIELYTRDEKRLSNV
FDIETMFREYNYYAQRVSGLRKDLLDLSTNRNQFVDAFGSLMDDLGAVGQTVVNA
VSGVATLFSSIVTGFINFIKNPFGGMLMIIVVIGVLFAIYFLTKKTKIYETAPIKMIYPEI
DKLKEREGKSEIAPISEEELERIVLAMHIHQQNSHMETKTRKDPKDSILTRAQNMLRK
RSGYSNLKNAESVEMLNTL

In some embodiments, the one or more engineered PLHV antigens contemplated by the present disclosure are variants of SEQ ID NO: 34. In some embodiments, the one or more engineered PLHV antigens contemplated by the present disclosure comprise the N terminus from the original GENBANK sequence labeled AMO03269.1. In some embodiments, the one or more engineered PLHV antigens can comprise SEQ ID NO: 35.

PLHV 1 antigen sequence-partial
SEQ ID NO: 35
MLFYIHIYKCTMAGSLKLRGSVLALWYLYQVALYSLSIAETGVTSPPNTATWSTESP
LTGHYGTHDSSHGERGNNENRDSEEQNKNIYGSPSTFPYRVCSASGVGDVFRFQTDH
VCPDASDMVHSEGILLIYKQNIIPFMFRVRKYRKVVTTSTVYNGIYSDSITNQHTFYK
SIEPWETEKMDTIYQCFNSLRLNTGGNLLTYVDRDDINMTVFLQPVDGVTPDVKRYG
SQPELYLEPGWFWGSYRRRTTVNCELMDMFARSNPPF
10Xhis tag-PLHV 1 antigen sequence -partial
SEQ ID NO: 36
MLFYIHIYKCTMAGSLKLRGSVLALWYLYQVALYSLSIAETGVTSPPNTATWSTESP
LTGHYGTHDSSHGERGNNENRDSEEQNKNIYGSPSTFPYRVCSASGVGDVFRFQTDH
VCPDASDMVHSEGILLIYKQNIIPFMFRVRKYRKVVTTSTVYNGIYSDSITNQHTFYK
SIEPWETEKMDTIYQCFNSLRLNTGGNLLTYVDRDDINMTVFLQPVDGVTPDVKRYG
SQPELYLEPGWFWGSYRRRTTVNCELMDMFARSNPPFHHHHHHHHHH

In another embodiment, the one or more engineered PLHV antigens can be PLHV-2 antigens comprising the amino acid sequence of SEQ ID NO: 37, 38, 39, or a combination thereof.

AAO12353.1|glycoprotein B(complete sequence) Porcine lymphotropic
herpesvirus 2
SEQ ID NO: 37
MLFYIHIYKCTMAGSLKLRRLALALWCQFQVALYALSKAEKSATSPANTQA
WSTEALPIGQYGTYDSSHGERATSENRDEEEHNKNIYGSPSTFPYRVCSASGVGDIFR
FQTDHVCPDASDMVHSEGILLIYKQNIIPFMFRVRKYRKVVTTSTVYNGIYSDSITNQ
HTFYKSIEPWETEKMDTIYQCFNSLRLNTGGNLLTYVDRDDINMTVFLQPVDGVTPD
VKRYGSQPDLYLEPGWFWGSYRRRTTVNCELMDMFARSNPPFDFFVTATGDTVEM
SPFWSGEDDHENKMNEKLWFLSVINDYKVVDYQNRGTVPLGKTRIFLDREEYTLSW
EKHLKNISYCPLTLWKAFYNGIQTEHSGSYHFVANDITASFTTSKEDMKDFNTTYHC
LNDEIKAEIEKKYAKVNSTHSRYGDLQYFKTDGGLFLVWQPLIQNRLLDAKNKLNN
ETVSKRSRRQVDSTTGPMMEATGNGAGGEYSSENSITVAQVQYAYDNLRTRINNILE
ELSKAWCREQHRAALMWNELSKINPTSVMSMIYNRPVSAKRIGDVISVSNCIVVDQT
SVSLHKSLRLLNAADDKCFSRPPVTFKFMNDSTIYKGQLGVNNEILLTTTYLETCQEN
TEYYFQAKTDMYIYKNYEHLKTVPLSSITTLDTFIALNFTLLENVDFKVIELYTRDEK
RLSNVFDIETMFREYNYYAQRVSGLRKDLLDLSTNRNQFVDAFGSLMDDLGAVGQT
VVNAVSGVATLFSSIVTGFINFIKNPFGGMLMIIVVIGVIFAIYFLTKKTKIYETAPIKMI
YPEIDKLKEREGKSEIQPISEEELERIILAMHIHQQNSHMETKARKDPKDTILTRAQNM
LRKRRGYTNLKNADSVEMLNTL

In some embodiments, the one or more engineered PLHV antigens contemplated by the present disclosure are variants of SEQ ID NO: 37. In some embodiments, the one or more engineered PLHV antigens contemplated by the present disclosure comprise the N terminus from the original GENBANK sequence labeled AA012353.1. In some embodiments, the one or more engineered PLHV antigens can comprise SEQ ID NO: 38.

PLHV2 antigen sequence-partial
SEQ ID NO: 38
MLFYIHIYKCTMAGSLKLRRLALALWCQFQVALYALSKAEKSATSPANTQAWSTEA
LPIGQYGTYDSSHGERATSENRDEEEHNKNIYGSPSTFPYRVCSASGVGDIFRFQTDH
VCPDASDMVHSEGILLIYKQNIIPFMFRVRKYRKVVTTSTVYNGIYSDSITNQHTFYK
SIEPWETEKMDTIYQCFNSLRLNTGGNLLTYVDRDDINMTVFLQPVDGVTPDVKRYG
SQPDLYLEPGWFWGSYRRRTTVNCELMDMFARSNPPFDFFVTATGDTVEMSPFWSG
EDDHENKMNEKLWF
10Xhistag-PLHV2 antigen sequence-partial
SEQ ID NO: 39
MLFYIHIYKCTMAGSLKLRRLALALWCQFQVALYALSKAEKSATSPANTQAWSTEA
LPIGQYGTYDSSHGERATSENRDEEEHNKNIYGSPSTFPYRVCSASGVGDIFRFQTDH
VCPDASDMVHSEGILLIYKQNIIPFMFRVRKYRKVVTTSTVYNGIYSDSITNQHTFYK
SIEPWETEKMDTIYQCFNSLRLNTGGNLLTYVDRDDINMTVFLQPVDGVTPDVKRYG
SQPDLYLEPGWFWGSYRRRTTVNCELMDMFARSNPPFDFFVTATGDTVEMSPFWSG
EDDHENKMNEKLWFHHH

In another embodiment, the one or more engineered PLHV antigens can be PLHV-3 antigens comprising the amino acid sequence of SEQ ID NO: 40, 41, 42, or a combination thereof. In some embodiments, the one or more engineered PLHV antigens can comprise the amino acid sequence of SEQ ID NO: 34, 35, 36, 37, 38, 39, 40, 41, and 42.

AAO12300.1|glycoprotein B (complete sequence) [Porcine
lymphotropic herpesvirus 3]
SEQ ID NO: 40
MLYSTHIYKCTMANSAKYRGCGQVLWMLFQVMFYFAVYGQYITTGAYDGNVNTE
ESEVTNHEYNYTDANTTAPEKKDPKEENQFYGSPSTFPYRVCSASGVGDIFRFQTEH
KCPDTNDMVHNEGILLIYKQNIVPFMFRVRKYRKVVTTSTVYNGIYSDSVTNQHTFY
KSIATWETEKMDTVYQCFNSLKLNVGNNLLTYVDRDDINMTVFLQPVDGVTTDVK
RYGSQPDLYLEPGWFWGSYRRRTTVNCELTDMFARSNPPFDFFVTATGDTVEMSPF
WTGPDDKENKLNEKPWFLTVIDGYKVVDYENRGTLPQGKTRIFLDREDYTLSWEKH
LKNVSYCPLTLWKSFHNGIQTVHSSSYHFVANDITASFTVDKEELKDENKTYSCLKD
EIQNEMQKKFEKINSTHSKVGEMQYFKTEGGLFIVWQPLIQNRLIEARSRLNNEALRR
VRRQTDGTSVTTTPEATVNNGNRTGANEESSENAISAAQVQYAYDNLRMRINNILED
LSKAWCREQHRAALMWNELSKINPTSVMTMIYNKPVSAKRIGDVISVSNCIIVDQNS
VSLHKSLRVPNSEERCFSRPPVTFKFTNDSTVYKGQLGVNNEILLTTTYLETCQENTE
YYFQAKKEMYVYKNYEHLKTVPLASITTLDTFIALNFTLLENVDFKVIELYTRDEKR
LSNVFDIETMFREYNYYTQRVSGLRKDLLDLSTNRNQFVDAFGSLMDDLGAVGQTV
VNAVSGVATLFSSIVTGFINFIKNPFGGMLIILVIIGVVVALYFLTKKTRHYEQAPVKM
IYPDIEKLKEREKNENLKPISQEELDRIILAMHTYQQNSLEQLKEKKAPTPGFLSKAQN
MLRKRSGYTNLVNSDSVEMLNSL

In some embodiments, the one or more engineered PLHV antigens contemplated by the present disclosure are variants of SEQ ID NO: 40. In some embodiments, the one or more engineered PLHV antigens contemplated by the present disclosure comprise the N terminus from the original GENBANK sequence labeled AA012300.1. In some embodiments, the one or more engineered PLHV antigens can comprise SEQ ID NO: 41.

PLHV3 antigen sequence-partial
SEQ ID NO: 41
MLYSTHIYKCTMANSAKYRGCGQVLWMLFQVMFYFAVYGQYITTGAYDGNVNTE
ESEVTNHEYNYTDANTTAPEKKDPKEENQFYGSPSTFPYRVCSASGVGDIFRFQTEH
KCPDTNDMVHNEGILLIYKQNIVPFMFRVRKYRKVVTTSTVYNGIYSDSVTNQHTFY
KSIATWETEKMDTVYQCFNSLKLNVGNNLLTYVDRDDINMTVFLQPVDGVTTDVK
RYGSQPDLYLEPGWFWGSYRRRTTVNCELTDMFARSNPPFDFFVTATGDTVEMSPF
WTGPDDKENKLNEKPWFLTVIDGYKVVDYENRGTLPQG
10Xhistag-PLHV3 antigen sequence-partial
SEQ ID NO: 42
MLYSTHIYKCTMANSAKYRGCGQVLWMLFQVMFYFAVYGQYITTGAYDGNVNTE
ESEVTNHEYNYTDANTTAPEKKDPKEENQFYGSPSTFPYRVCSASGVGDIFRFQTEH
KCPDTNDMVHNEGILLIYKQNIVPFMFRVRKYRKVVTTSTVYNGIYSDSVTNQHTFY
KSIATWETEKMDTVYQCFNSLKLNVGNNLLTYVDRDDINMTVFLQPVDGVTTDVK
RYGSQPDLYLEPGWFWGSYRRRTTVNCELTDMFARSNPPFDFFVTATGDTVEMSPF
WTGPDDKENKLNEKPWFLTVIDGYKVVDYENRGTLPQGHHHHHHHHHH

### V. Other Virus Assays and Multiple Virus Assays

Methods for detecting PLHV and PCMV are described herein, but the methods can also be applied to other viruses of interest using the same general techniques and methods and achieve the same benefits, e.g., detection of latent infections. For example, the assays described herein can be used to detect any porcine virus.

In some embodiments, the porcine virus can be enteric alpha coronavirus (SeACoV), porcine epidemic diarrhea virus, African swine fever virus (ASFV), Classical swine fever virus (CSFV), Foot-and-mouth disease virus, Porcine reproductive and respiratory syndrome virus (PRRSV), porcine circovirus type 2 (PCV2), porcine circovirus type 3 (PCV3), porcine circovirus type 4 (PCV4), porcine circovirus-associated diseases (PCVAD), swine hepatitis E virus (swine HEV), swine influenza virus (SIV), porcine Torque teno sus virus (TTSuV), porcine Sapovirus (Poricne SaV), Senecavirus A (SVA), high pathogenicity porcine epidemic diarrhea virus (PEDv), influenza virus H1N1pdm09, porcine enteroviruses, porcine toroviruses (PTOV), porcine sapelovirus (PSV), porcine bocavirus (PBoV), porcine kobuvirus (PKBV), SENECA virus (SVA), atypical porcine pestivirus (APPV), swine acute diarrhea syndrome (SADS coronavirus); SADS-COV, influenza D, porcine hemagglutinating encephalomyelitis coronavirus (PHE-COV); classical swine fever virus (CSF); porcine torovirus (PTOV); porcine respiratory reproductive syndrome virus (PRRSV); human influenza A; Nipah virus; atypical porcine pestivirus (APPV); porcine epidemic diarrhea coronavirus (PEDV); and porcine delta coronavirus (PDCOV); porcine epidemic diarrhea coronavirus (PED-CoV); coronaviruses transmissible gastroenteritis virus (TGEV); hepatitis E virus (HEV); Porcine bocavirus; Sapovirus; Sapelovirus; Posavirus-1; Porcine astrovirus; Porcine enterovirus-9; Kobuvirus; Porcine bocavirus-2; Porcine enterovirus-9; Coronavirus; Po-circo-like, or Porcine bocavirus-4; Porcine encephalomyocarditis virus (different from Porcine hemagglutinating encephalomyelitis (coronavirus)); Rotavirus A, B, or C; Pseudorabies virus (PHV-1); Vesicular stomatitis; Porcine parvovirus 1 or 2; Porcine adenovirus; Porcine respiratory corona virus; or porcine pneumoviruses, including swine Orthopneumovirus (SOV).

In some embodiments, the virus can be Adenoviridae, Anelloviridae, Astroviridae, Caliciviridae, Circoviridae, Parvoviridae, Picornaviridae, or Reoviridae.

The methods can be performed serially or in parallel. For example, in some embodiments, a single assay can be performed that detects more than one virus, if present, e.g., PLHV and PCMV.

On aspect of the present disclosure provides a method for detecting the presence of multiple viruses in a biological sample from a subject, the method can comprise obtaining a serum sample from the subject, diluting the serum sample with a diluent, contacting the diluted serum sample with antigens for each of the viruses of interest for a time period sufficient to allow binding between the antigens and corresponding antibodies in the diluted serum sample, and detecting the presence or absence of antibodies from the diluted serum sample reacting with the antigens using an automated capillary electrophoresis (CE) western or western blot analysis. The methods described herein can be used by a skilled artisan employing multiple viral antigens and detecting the corresponding antibodies, if any. Thus, the methods herein are not limited to a single virus or even PCMV and PLHV. Similarly, one of ordinary skill in the art can use multiple antigens for the same virus in a single assay. This is possible in the assay described herein because CE western techniques can provide additional information about the target protein, such as molecular weight estimation (e.g., as size, and size distributions) and protein intactness (e.g., isomers, fragments, and aggregates).

In some embodiments, the automated capillary electrophoresis (CE) western is a closed-loop automated capillary-based immunoassay system. In some embodiments, the samples are electrophoretically separated by weight. In some embodiments, separated proteins are immobilized on a solid support. In some embodiments, the solid support is a capillary wall of a microfluidic device. In some embodiments, electrophoretically separating the proteins from the sample can provide a size distribution profiling of the proteins found in the sample thereby allowing the detection of one or more viral antigens.

Another aspect of the present disclosure provides a kit for detecting an anti-porcine cytomegalovirus (PCMV) antibody in a biological sample from a subject. The kit can comprise: one or more engineered PCMV antigens as described herein; and a detection entity for detecting the interaction between the one or more engineered PCMV antigens and the one or more anti-PCVM antibodies present in a biological sample.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further include or incorporate the variations of any or all other variations, aspects or embodiments of the present technology.

### Example 1: PCMV Serology Western Blot Assay

Many PCR-based methods have been developed by academic and diagnostic laboratories to detect PCMV infection in a subject. These methods are laborious and have only been able to detect a viral infection by a zoonotic virus only when the virus is in an active viremic stage. After the virus goes into latency stage, PCR-based assays were often prone to giving false negative results. Immunologic methods like ELISA or western blot can serve as a reliable alternative to PCR based methods. Western Blot can detect infection in the viremic as well as in latent stage. However, to this date, the diagnostic laboratories have not offered or do not offer PCMV serology assays. Moreover, all published PCMV serology assays are cumbersome, less sensitive, and unreliable. Hence, a rapid high-throughput PCMV serology western blot technique using the Simple Wes™ instrument to detect PCMV anti-IgG antibodies in a sample from a subject was developed. The novel automated serology assay described herein includes the use of porcine serum or a serum from a subject transplanted with a porcine cell, tissue or organ, the application of specific dilutions and antigen concentrations, and unique modifications to the Wes™ protocols. Together, these modifications (i.e., optimizations) showed a consistent and reliable detection of PCMV anti-IgG antibodies.

The following example validated a PCMV serology western blot assay with known PCMV positive, negative, and latent phase infection samples.

### Methods

PCMV antigen preparation: Porcine CMV glycoprotein B (Accession Number: FJ595497.1; 2309-2771) was expressed as a 10×His-tag protein (SEQ ID NO: 33) in bacterial expression vector. Cloning DNA sequence, protein purification and quality control of the antigen was performed by a contract manufacturer (Origene technologies Inc). Rabbit anti-His primary antibody (assay positive control) was used to determine the expected molecular weight of the PCMV antigen (17 kDa and 40 kDa).

Study subjects: 9 age-matched pigs (60-116 days) were included in the study, of which five, were early weaned from the sow and stayed behind a biosecure barrier throughout their life. These five animals were PCMV negative by previous PCR analysis. The other four pigs were raised in the general herd and were shown to be PCMV positive by previous PCR analysis. four older pigs (238,240,317 and 323 days) that were PCMV positive by previous PCR analysis were also included in the analysis. Whole blood was obtained for PCR and PCMV serology western blot assay from each of these porcine animals.

PCMV serology western blot setup: The PCMV antigen was diluted to 0.29 μg/μl in PBS buffer and 0.52 g was loaded per lane following the manufacturers (Simple Wes™) protocol for sample preparation protocol. Porcine sera were diluted to 1:75; 1:300; and 1:1200 in serum dilution buffer. Rabbit anti-pig IgG HRP secondary antibodies were diluted to 1:250 in antibody diluent buffer. All reagents were loaded into the Simple Wes™ 22-250 kDa module plate. Serum incubation with the antigen time was increased to 60 minutes independent from the manufacturer's recommendations. PCR analysis was done at a diagnostic laboratory or in-house (data not shown).

### Results

Samples from Pigs Raised in a Biosecure High Herd Health Barn Did not Show any Detectable Antibodies to PCMV.

Serum samples from pigs (#1-5) that were raised in a biosecure high herd health barn (i.e., the barrier) and were PCMV negative by previous PCR analysis and a known PCMV positive pig (#6) were subjected to PCMV serology western blot analysis. No signal was detected in serum samples from the five (5) pigs that were raised in the high herd health barn while the serum sample from a known PCMV PCR-positive pig showed a strong signal at 17 kDa and 40 kDa up to 1:1200 dilution. PCMV serology western blot analysis matched PCR results (data not shown) indicating that the serology assay could discriminate PCMV positive and negative pigs.

FIGS. 2-4 show western blot results using the porcine cytomegalovirus (PCMV) serology western blot assay. The experiments were performed using sera from six different porcine animals that were raised in two different conditions and demonstrated the detection of anti-PCMV antibodies in porcine animal that were raised outside the biosecure high herd health barn (i.e., the barrier) facility when compared to their littermates that were raised inside the biosecure high herd health barn (i.e., the barrier) facility. The positive control contained rabbit anti-His primary antibody that was used to determine the expected molecular weight of the PCMV antigen (17 kDa and 40 kDa). In particular, sample #1, #2, and #3 were pig sera samples obtained from three different pigs that were raised inside the biosecure high herd health barn (i.e., the barrier) facility. As shown in FIGS. 2-4, samples from all three pigs did not show any detectable antibodies to PCMV.

Samples from Pigs Raised Outside a Biosecure High Herd Health Barn Showed Detectable Antibodies to PCMV.

PCMV serology western blot was performed on serum samples from pigs (#6-9) and older pigs (#10-13) all of which were raised in the general herd and were PCMV positive by previous PCR DNA analysis (data not shown). A serum sample from a known PCMV PCR-positive (#6) was also included in the assay (data not shown). Like the positive control, all pigs (6-9, 10-13) showed positive signals at the expected molecular weight indicating positivity for PCMV infection. However, the PCR assay was negative for PCMV indicating that the infection was in the latent phase (data not shown). These data demonstrated that the novel serology assay was sensitive enough to detect latent PCMV virus in all tested pigs, including older pigs.

FIGS. 2-4 show results from experiments performed using sera from six different porcine animals that were raised in two different conditions. Samples #4, #5, #6 were pig sera samples derived from three different pigs that were raised outside the barrier facility and littermates to pigs #1, #2, and #3 described above. As shown in FIGS. 2-4, all three samples showed detectable antibodies to PCMV at 1:75 and 1:300 dilutions. The negative control contained no serum. The positive control contained rabbit anti-His primary antibody that was used to determine the expected molecular weight of the PCMV antigen (17 kDa and 40 kDa).

### Example 2: Sensitivity of the PCMV Serology Western Blot Assay Compared to PCR-Based Assay

To determine the sensitivity of the PCMV serology western blot assay, timeline experiments were performed to detect anti-PCMV antibodies at different stages of infection in a pig that was raised outside a biosecure barrier facility using a PCR-based assay and the PCMV serology western blot assay. This example shows that the PCMV serology western blot assay was as sensitive or more sensitive than the PCR-based assay.

Serum samples were collected from the pigs between 12 and 103 days of age and tested for PCMV at an independent veterinary diagnostic laboratory via PCR-based assay and PCMV serology western blot assay. FIG. 5 shows western blot results generated by Compass software of a porcine cytomegalovirus (PCMV) serology western blot assay. In 12-day old animal, two weak positive signals were detected at the correct MW. Stronger signals were observed in 83 day-old animals and the strength of the signals was enhanced in 88-day old animal and 103-day old animals. The positive control contained serum from a pig known to be PCMV positive by PCR analysis from an independent veterinary diagnostic laboratory and was used to determine the expected molecular weight of the PCMV antigens. The negative control contained no serum (e.g., buffer only).

Table 1 summarizes timeline of results obtained by independent veterinary diagnostic lab and PCMV serological western blot assay. At day 12, PCR results from the independent veterinary diagnostic lab were negative. However, the PCMV serology western blot assay showed a weak positive signal. This weak signal could be passively transmitted maternal antibodies. Thus, the data demonstrate that the PCMV serology western blot assay can detect passively transmitted maternal antibodies.

At day 83 and day 88, PCR results from the independent veterinary diagnostic lab were positive for PCMV. At day 83 and day 88, PCR analysis from the independent veterinary diagnostic lab and PCMV serology western blot assay both showed positive PCMV signals. At day 103, the PCMV serology western blot assay continued to show a positive result. Together, Table 1 and FIG. 5 demonstrate the ability to detect anti-PCMV antibodies during early stage PCMV viremia.

The table demonstrates timeline of testing with relevant results for the PCR analysis from independent veterinary diagnostic lab and the PCMV serology western blot assay and exemplifies the importance of the PCMV serology western blot assay for detecting maternal antibodies and antibodies produced during early-stage viremia.

### Example 3: The PCMV Serology Western Blot Assay can Detect the Presence of PCMV in Latent Stage PCMV Infection

To further assess the sensitivity of the PCMV serology western blot assay, the ability of the PCMV serology western blot assay to detect latent stage infection was determined using animals raised in a biosecure barrier facility.

Twenty-one porcine animals that were raised in a biosecure barrier facility were tested by PCR-based assay. All twenty-one animals received a negative PCMV PCR result from an independent veterinary diagnostic lab. These animals were then retested using the PCMV serology western blot assay. FIG. 6 shows western blot results generated by Compass software illustrating the effectiveness and sensitivity of the porcine cytomegalovirus (PCMV) serology western blot assay. It demonstrates the detection of anti-PCMV antibodies in latent phase of PCMV infection in two out of twenty-one porcine animals that were raised in a biosecure barrier facility (sample #12 and #19).

As shown in FIG. 6, 19 retested animals were also negative for PCMV using the PCMV serology western blot assay. Surprisingly, two out of twenty-one porcine animals were positive for PCMV (sample #12 and #19). Sample #12 was >3 months of age and sample #19 was 14 months of age.

These data demonstrate that the PCMV serology western blot assay was able to robustly detect PCMV in latent stage across the lifespan of the animals. Furthermore, the PCMV serology western blot assay was able to robustly detect latent PCMV infection that was undetectable by PCR.

### CONCLUSION

This is the first demonstration of a PCMV serology western blot assay using the Simple Wes™. Thus, the PCMV serology western blot assay has never been done. The results described herein provided a novel serology assay that can potentially be run at any diagnostic laboratory under GLP conditions.

Each assay can process at least about 24 samples with a turnaround time of 4-5 hours to produce final results compared to at least 8-24 hours for traditional western blot. Furthermore, the novel serology assay disclosed herein can detect PCVM antibodies at various life stages of any subject (e.g., pigs). For example, the assay disclosed herein can detect anti-PCVM antibodies in the colostrum, in newborn piglets, in potentially infected young piglets (e.g., about 2-3 months old), in donor pigs for xenotransplantation (e.g., >4 months old), in young sows (e.g., >7 months old), in boars (e.g., >12 months old) and/or mature breeding sows (e.g., >12 months old).

Furthermore, this novel PCMV serology western blot assay is a qualified assay for detecting anti-PCMV antibodies in preclinical pig sera samples that were bound for a non-human primate (NHP) or a human xenotransplantation.

Table 2 shows the number of pig sera samples that have been assayed for the presence of anti-PCMV antibodies using this novel PCMV serology western blot assay.

TABLE 2
Number of pig sera samples assayed for
the presence of anti-PCMV antibodies

| | Number of pig sera | Number of pig sera |
|---|---|---|
| Total Number of pig | samples tested | samples tested |
| sera samples run | positive PCMV | negative for |
| using this assay | antibodies | PCMV antibodies |
| 322 | 86 | 236 |

### Example 4: PLHV Serology Assays

PCR-based methods have been developed by academic and diagnostic labs to detect gamma herpes virus and PLHV infection. These methods are laborious and can only detect the virus is in an active viremic stage. After the virus goes into latency, the assays give false negative results. To this date, none of the veterinary diagnostic laboratories have validated PLHV-1, PLHV-2, and PLHV-3 serology assays. PLVH-1, PLHV-2 and PLHV-3 serology assays published in peer reviewed literature are cumbersome, less sensitive, and unreliable. Novel PLHV-1, PLHV-2, and PLHV-3 serology western blot assays were designed and validated using known positive and negative PLHV infected serum samples to detect the presence of PLHV-1, PLHV-2, and PLHV-3 IgG antibodies in pig sera.

### Methods

### PLHV1 Antigen Preparation

Porcine lymphotropic herpesvirus 1 GD33 envelope glycoprotein B (Accession Number: KT844939.1; 1-798) was expressed as a 10×His-tag protein (SEQ ID NO: 33) in bacterial expression vector. Cloning DNA sequence, protein purification and quality control of the antigen was done by a contract manufacturer (Origene Technologies Inc). Rabbit anti-His primary antibody (assay positive control) was used to confirm the expected protein molecular weight.

### PLHV2 Antigen Preparation.

Porcine lymphotropic herpesvirus-2 glycoprotein B (Accession Number: NC_038265.1; 1-897) was expressed as a 10×His-tag protein (SEQ ID NO: 33) in bacterial expression vector. Cloning DNA sequence, protein purification and quality control of the antigen was done by a contract manufacturer (Origene technologies Inc). Rabbit anti-His primary antibody (assay positive control) was used to confirm the expected protein molecular weight.

### PLHV3 Antigen Preparation.

Porcine lymphotropic herpesvirus-2 glycoprotein B (Accession Number: NC_0552341.1; 1-951) was expressed as a 10×His-tag protein (SEQ ID NO: 33) in bacterial expression vector. Cloning DNA sequence, protein purification and quality control of the antigen was done by a contract manufacturer (Origene Technologies Inc). Rabbit anti-His primary antibody (assay positive control) was used to confirm the expected protein molecular weight.

### PLHV Serology Western Blot Setup

PLHV-1, PLHV-2, and PLHV-3 antigens are diluted to 0.29 μg/μl in PBS buffer and 0.52 μg are loaded per lane following manufacturers protocol for sample preparation protocol. Porcine sera are diluted to 1:37.5; 1:75; 11:300, or 1:1200 in serum dilution buffer. Rabbit anti-pig IgG HRP secondary antibodies are diluted to 1:2400 in antibody diluent buffer. All reagents are loaded into the Simple WES™ 22-250 kDa module plate. The serum incubation with antigen time are increased to 60 or 90 minutes. The increased incubation time is a deviation from the manufacturer's recommendations.

PLHV-1, PLHV-2, and/or PLHV-3 serology western blot Assays using the Simple WES™ have never been performed before. The combination of the Simple WES™, the novel antigen designs, and the increased incubation time can provide a novel serology system that can be run at any Veterinary Diagnostic laboratory (VDL) under GLP conditions. Each assay can process at least about 24 samples with a turnaround time of 4-5 hours to produce final results when compared to at least 8-24 hours. Furthermore, the novel serology assays can detect PLHV-1, PLHV-2, PLHV-3 antibodies at various life stages of any subject (e.g., pigs). For example, the assay disclosed herein can detect maternal anti-PLHV-1, -PLHV-2, and/or -PLHV-3 antibodies in the colostrum, in newborn piglets, in potentially infected young piglets (e.g., about 2-3 months old), in donor pigs for xenotransplantation (e.g., >4 months old), in young sows (e.g., >7 months old), in boars (e.g., >12 months old) and/or mature breeding sows (e.g., >12 months old).

### Results

Samples from Pigs Raised Outside a Biosecure High Herd Health Barn Showed Detectable Antibodies to PLHV1.

FIG. 7 shows western blot results generated by Compass software illustrating the effectiveness and sensitivity of a porcine lymphotropic virus (PLHV)-1 serology western blot assay. The experiments were performed using sera from three different porcine animals that were raised in two different conditions and demonstrate the detection of anti-PLHV-1 antibodies in porcine animal that were raised outside the biosecure high herd health barn (i.e., the barrier) facility when compared pigs that were raised inside the biosecure high herd health barn (i.e., the barrier) facility. In particular, sample #1, and #2, are pig sera samples obtained from 2 different pigs that were raised inside the biosecure high herd health barn (i.e., the barrier) facility and sample #3 is the pig sera sample that was obtained from the pig that was raised outside the biosecure high herd health barn.

As shown in FIG. 7, anti-PLHV-1 antibodies were detected in a porcine animal raised outside a biosecure barrier facility (sample #3; infected), but not in porcine animals raised inside a biosecure barrier facility (sample #1 and #2). Samples from 2 pigs did not show any detectable antibodies to PLHV1, whereas sample #3 pig sera showed detectable PLHV1 antibodies.

Samples #1 and #2 also had a negative PLHV PCR result from an independent veterinary diagnostic lab, while sample #3 had a positive PLHV PCR result. Thus, as expected, samples from 2 pigs did not show any detectable antibodies to PLHV1 whereas sample #3 pig sera show detectable PLHV1 antibodies.

The engineered PLHV-1 antigen was detected using a rabbit anti-histidine (His) primary antibody and anti-pig IgG HRP conjugate. The positive control contained the rabbit anti-His primary antibody and was used to determine the expected molecular weight of the PLHV-1 antigen. The tested PLHV-1 antigen had a molecular weight of about 31 kDa. The negative control contained no serum.

Samples from Pigs Raised Outside a Biosecure High Herd Health Barn Showed Detectable Antibodies to PLHV2.

FIG. 8 shows western blot results generated by Compass software illustrating the effectiveness of a porcine lymphotropic virus (PLHV)-2 serology western blot assay. The experiments were performed using sera from three different porcine animals that were raised in two different conditions and demonstrate the detection of anti-PLHV-2 antibodies in porcine animal that were raised outside the biosecure high herd health barn (i.e., the barrier) facility when compared pigs that were raised inside the biosecure high herd health barn (i.e., the barrier) facility. In particular, sample #1, and #2, are pig sera samples obtained from 2 different pigs that were raised inside the biosecure high herd health barn (i.e., the barrier) facility and sample #3 is the pig sera sample that was obtained from the pig that was raised outside the biosecure high herd health barn.

As shown in FIG. 8, anti-PLHV-2 antibodies were detected in a porcine animal raised outside a biosecure barrier facility (sample #3; infected), but not in porcine animals raised inside a biosecure barrier facility (sample #1 and #2). Samples #1 and #2 had a negative PLHV PCR result from an independent veterinary diagnostic lab, while sample #3 had a positive PLHV PCR result. As expected, samples from 2 pigs did not show any detectable antibodies to PLHV2, whereas sample #3 pig sera showed detectable PLHV2 antibodies.

The engineered PLHV-2 antigen was detected using a rabbit anti-histidine (His) primary antibody and anti-pig IgG HRP conjugate. The positive control contained the rabbit anti-His primary antibody and was used to determine the expected molecular weight of the PLHV-2 antigen. The tested PLHV-2 antigen had a molecular weight of about 41 kDa. The negative control contained no serum.

Samples from Pigs Raised Outside a Biosecure High Herd Health Barn Showed Detectable Antibodies to PLHV3.

FIG. 9. shows western blot results generated by Compass software illustrating the effectiveness of a porcine lymphotropic virus (PLHV)-3 serology western blot assay. The experiments were performed using sera from three different porcine animals that were raised in two different conditions and demonstrate the detection of anti-PLHV-3 antibodies in porcine animal that were raised outside the biosecure high herd health barn (i.e., the barrier) facility when compared pigs that were raised inside the biosecure high herd health barn (i.e., the barrier) facility. Sample #1, and #2, are pig sera samples obtained from 2 different pigs that were raised inside the biosecure high herd health barn (i.e., the barrier) facility and sample #3 is the pig serum sample that was obtained from the pig that was raised outside the biosecure high herd health barn.

As shown in FIG. 9, anti-PLHV-3 antibodies were detected in a porcine animal raised outside a biosecure barrier facility (sample #3; infected), but not in porcine animals raised inside a biosecure barrier facility (sample #1 and #2). Samples #1 and #2 had a negative PLHV PCR result from an independent veterinary diagnostic lab, while sample #3 had a positive PLHV PCR result. As expected from the PCR results, samples from 2 pigs did not show any detectable antibodies to PLHV3 whereas sample #3 pig serum showed detectable PLHV3 antibodies.

The engineered PLHV-3 antigen was detected using a rabbit anti-histidine (His) primary antibody and anti-pig IgG HRP conjugate. The positive control contained the rabbit anti-His primary antibody and was used to determine the expected molecular weight of the PLHV-3 antigen. The tested PLHV-3 antigen had a molecular weight of about 40 kDa. The negative control contained no serum.

### The PCR Test Results Did not Distinguish Between PLHV-1, PLHV-2, and PLHV-3

The serology western blot assay was more sensitive than the PCR assay because it distinguished between PLHV-1, PLHV-2, and PLHV-3 (See FIGS. 7, 8, and 9). For example, the tested PLHV-1 antigen had a molecular weight of about 31 kDa (FIG. 7). The tested PLHV-2 antigen had a molecular weight of about 41 kDa (FIG. 8). The tested PLHV-3 antigen had a molecular weight of about 40 kDa (FIG. 9). In contrast, the PCR-based assay performed by an independent veterinary diagnostic laboratory did not distinguish between PLHV-1, PLHV-2, and PLHV-3. These results further demonstrate the sensitivity of the novel serology western blot assay over the methods used in the art for the detection of PLHV-1, PLHV-2, and PLHV-3.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting. In addition, where features or aspects of the disclosure are described in terms of Markush groups, those skilled in the art will recognize that the disclosure is also thereby described in terms of any individual member or subgroup of members of the Markush group.

What is claimed is:

1. A method for detecting a porcine cytomegalovirus (PCMV) or a porcine lymphotropic herpesvirus (PLHV) in a biological sample from a subject, comprising obtaining a serum sample from the subject, diluting the serum sample with a diluent, contacting the diluted serum sample with a PCMV antigen or a PLHV antigen for a time period sufficient to allow binding between the PCMV antigen or the PLHV antigen and a corresponding antibody in the diluted serum sample, detecting the presence or absence of an antibody from the diluted serum sample that reacts with the PCMV antigen or the PLHV antigen using Western blot analysis.

2. A method for detecting an anti-porcine cytomegalovirus (PCMV) antibody or an anti-porcine lymphotropic herpesvirus (PLHV) antibody in a biological sample from a subject, the method comprising:
(a) introducing into one or more capillaries of a microfluidic device one or more engineered PCMV or PLHV antigens; a biological sample from the subject, one or more anti-immunoglobulin antibodies, and one or more chemiluminescent molecules;
(b) separating the one or more engineered PCMV or PLHV antigens electrophoretically;
(c) immobilizing the one or more electrophoretically separated engineered PCMV or PLHV antigens on a capillary wall;
(d) contacting the one or more immobilized engineered PCMV or PLHV antigens with the biological sample from the subject;
(e) incubating the one or more immobilized engineered PCMV or PLHV antigens with the biological sample for about 10 minutes to about 120 minutes; and
(f) detecting the binding of the one or more engineered PCMV or PLHV antigens to the biological sample by immunodetection and/or chemiluminescent detection,
wherein the presence of an immunodetection signal and/or a chemiluminescent signal indicates the presence of one or more anti-PCMV or anti-PHLV antibodies in the biological sample.

3. The method of claim 2, wherein the method is an automated serological method for detecting the anti-PCMV or anti-PLHV antibody in a porcine animal.

4. The method of claim 3, wherein the one or more engineered PCMV or PHLV antigens are electrophoretically separated by weight and immobilized on a solid support.

5. The method of claim 4, wherein the solid support is a capillary wall of a microfluidic device.

6. The method of claim 2, wherein the method is performed in a closed-loop automated capillary-based immunoassay system.

7. The method of claim 2, wherein the one or more engineered PCMV or PHLV antigens with the biological sample are incubated for about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 100 minutes, about 105 minutes, about 110 minutes, about 115 minutes, or about 120 minutes.

8. The method of claim 2, wherein the anti-PCMV or the anti-PHLV antibody is an IgM or an IgG antibody.

9. The method of claim 2, wherein the one or more engineered PCMV or PHLV antigens are selected from the group consisting of envelope glycoprotein B (gB), envelope glycoprotein H (gH), envelope glycoprotein L (gL), envelope glycoprotein M (gM), envelope glycoprotein N (gN), major tegument phosphoprotein1 (U54A); major tegument phosphoprotein2 (U54B); and U100p.

10. The method of claim 2, wherein the one or more engineered PCMV or PHLV antigens comprise glycoprotein B (gB).

11. The method of claim 10, wherein:
(a) the one or more engineered PCMV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 2, 3, 8, 9, 25, 26, 27, 28, 29, 30, 31, or 32; or
(b) the one or more engineered PLHV antigens comprise an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 34, 35, 36, 37, 38, 39, 40, 41, 42, or a combination thereof.

12. The method of claim 10, wherein the one or more engineered PCMV antigens comprise:
(a) the amino acid sequence of SEQ ID NO: 2, 3, 8, 9, 25, 26, 27, 28, 29, 30, 31, 32, or a combination thereof;
(b) the amino acid sequence of SEQ ID NO: 3 or 32; or
(c) the amino acid sequence of SEQ ID NO: 2, 3, 8, 9, 25, 26, 27, 28, 29, 30, 31 and 32.

13. The method of claim 2, wherein the one or more engineered PCMV antigens are encoded by:
(a) a polynucleotide sequence selected from SEQ ID NO: 1, 5, 6, 7, 10, 11, or a combination thereof; or
(b) a polynucleotide sequence selected from SEQ ID NO: 17, 21, or a combination thereof.

14. The method of claim 2, wherein the one or more engineered PCMV antigens comprise glycoprotein gH and/or glycoprotein gL.

15. The method of claim 14, wherein the one or more engineered PCMV antigens comprise:
(a) an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 18 or 22; or
(b) the amino acid sequence of SEQ ID NO: 18 and/or 22.

16. The method of claim 2, wherein the one or more engineered PCMV antigens comprise U100p (gQ).

17. The method of claim 2, wherein the subject is a mammal selected from a primate, a non-human primate, a human, or a porcine animal.

18. The method of claim 2, wherein the porcine lymphotropic herpesvirus (PLHV) is selected from PLHV-1, PLHV-2, PLHV-3, or a combination thereof.

19. The method of claim 2, wherein:
(a) the one or more engineered PHLV antigens comprise the amino acid sequence of SEQ ID NO: 34, 35, 36, 37, 38, 39, 40, 41, 42, or a combination thereof; or
(b) the one or more engineered PLHV antigens comprise the amino acid sequence of SEQ ID NO: 34, 35, 36, 37, 38, 39, 40, 41, and 42.

20. The method of claim 2, wherein the one or more engineered PLHV antigens is one or more
(a) PLHV-1 antigens comprising the amino acid sequence of SEQ ID NO: 34, 35, 36, or a combination thereof;
(b) PLHV-2 antigens comprising the amino acid sequence of SEQ ID NO: 37, 38, 39, or a combination thereof; and/or
(c) PLHV-3 antigens comprising the amino acid sequence of SEQ ID NO: 40, 41, 42, or a combination thereof.


SEQUENCE LISTING (48 sequences)

SEQ ID NO: 1 - DNA, 2580 bp, Suid betaherpesvirus 2
  source 1..2580: mol_type = genomic DNA
atgacagtgagcagtcggaatttattccggattgttttgttgattaactatggctatctcgtgtcatctaattctacgacatctacaacttcggcatctgaatcatcggttaccaccggatctcccggcactgtcagttctgaaacagcgacaccggaagtttctacggcttttgtgaacattaccggaaatttttcacaggaatacactgaagcaagcgatgatgaaaaatatccttttcgtgtatgcaatatggccgttggaacagatctatatagatttgataattatatcacttgtaataagtataatacggagactcagtattctgaaggtattttgttattgttcaagaaaaatatagttccgcacactttttttgttaggacgtataccaaggaattgtcgtttcaaacgacgtatagagacgttcatgtcatctatcttgttgataggccttcatataaagtgcccgttcctgtagatgaagctggatatataaatttgaatggtcaatgtttttccgctgccgagattagaaaccagggaataaattatcgggtatatcataaagatgataatacaaacaatacaatgagattatatcgtttgaaatttggatctaccattaatactcgttacatatccacgcctgaatttcagttcacatatggaacccattggttatataaaagctcatcttctattaattgcatagtaactgatactattggtaaatctgattacccatatgagaattttatactcggtacaggtgaatcggtggagatatcaccattttttaatggaacttctaaagaggtatttaatgaacagatgtactatttttcaatgaagagcaattacaccatgttagaaaaattagacgaacctaatggaccaaagaaaactatccctacgattgcttttttacaaaagggtgacacattgttttcatgggaggttaaggaacagactaattctcattgcaaatatacagcatggacaaaaaaacaccatgcattgcgagcagacatgacaaatagttatcattttatgatgaaggacatgacagcaacaatggtaacaacaaagaacactataaatcttacgggcaccgaatacgagtgtgtgaaaaatgatattgaaaaatatattacagatacgtttcagagcaaatataacaatacacacaacaaaacagaaaactatagtgtttatgaaacaactacaggtctaatattagtttggcagcctattgttaggaaatcaattaaagagttgaaagaatttatcaatgaaacccaacatactcgttataaacgagaaattgatggaagtgactcacttgtttacgctagtttgcagtatatgtacgatgctttgagagagtatataaatgctggtttcgcccagatagctgaggcgtggtgcgaagatcagaaaagaaccaatgaagttctttccgaattagctaaaataaatccatctaatgtcatgtctgtaatctacgataaatcattatccgcaaaattagttggagatgcaatttccgtatcttcgtatgtcaatgtaaatcagtctacggtgaaggtgcataaagatatgagaatatacgccaatgggacggcgaatagggagacgtgtttttctcgcccggtcgttacgtttgaattttcgaataataattcagttcaacaatttacgggtcaattggggccgaggaatgagattctgttaggcacgtatagagtggaaaaatgtgaacgtaacagtgttaaagttttttttgcgggtaaggaggcgtattttttttacaattacatttacacgaaaacggttaatatatctgatatcaatgtcgtagatacttttatccaccttaatataaagccgttagagaatacagactttgaggtattgcgaatgtattctaaaaatgagttagtgcaggctaacatatttgatctggaaagtcttttgagagatattaattcatataggagtgcgctttataatattgaatctaggatagctccaaggaaaccagattacgtctcaggtgtggattcttttctgcatgctcttggtataggtgcacccggagggttgggtgccgctctgggtatggcgacgggggcggtcacagatttcgtgactggcattttttccttctttaagaatccattcgggggacttttttccatgctgttttttgttttgcttgttttcttgatattttccgtgtattacagacagaagaatatatatacaaacccggtaggtgcgttgtttccgtatgcaaatagttcttcgggaactgcaatatctaatacgcattcctattatgagacagacaataaacaggagtttgagaatgacaggaaacctgatacgtccaatgccgtctctgagggcagtgccaataagtactctcaagaagatgccgtctgcatgttaatggctataaagaacctgggggatgcgtacaggcggaagaatgccacaaagcctagcccgagcgtattagataagatacgtcacttggaatatcagcagttatccaccgaggacgtgtga

SEQ ID NO: 2 - protein, 859 aa, synthetic construct
MTVSSRNLFRIVLLINYGYLVSSNSTTSTTSASESSVTTGSPGTVSSETATPEVSTAFVNITGNFSQEYTEASDDEKYPFRVCNMAVGTDLYRFDNYITCNKYNTETQYSEGILLLFKKNIVPHTFFVRTYTKELSFQTTYRDVHVIYLVDRPSYKVPVPVDEAGYINLNGQCFSAAEIRNQGINYRVYHKDDNTNNTMRLYRLKFGSTINTRYISTPEFQFTYGTHWLYKSSSSINCIVTDTIGKSDYPYENFILGTGESVEISPFFNGTSKEVFNEQMYYFSMKSNYTMLEKLDEPNGPKKTIPTIAFLQKGDTLFSWEVKEQTNSHCKYTAWTKKHHALRADMTNSYHFMMKDMTATMVTTKNTINLTGTEYECVKNDIEKYITDTFQSKYNNTHNKTENYSVYETTTGLILVWQPIVRKSIKELKEFINETQHTRYKREIDGSDSLVYASLQYMYDALREYINAGFAQIAEAWCEDQKRTNEVLSELAKINPSNVMSVIYDKSLSAKLVGDAISVSSYVNVNQSTVKVHKDMRIYANGTANRETCFSRPVVTFEFSNNNSVQQFTGQLGPRNEILLGTYRVEKCERNSVKVFFAGKEAYFFYNYIYTKTVNISDINVVDTFIHLNIKPLENTDFEVLRMYSKNELVQANIFDLESLLRDINSYRSALYNIESRIAPRKPDYVSGVDSFLHALGIGAPGGLGAALGMATGAVTDFVTGIFSFFKNPFGGLFSMLFFVLLVFLIFSVYYRQKNIYTNPVGALFPYANSSSGTAISNTHSYYETDNKQEFENDRKPDTSNAVSEGSANKYSQEDAVCMLMAIKNLGDAYRRKNATKPSPSVLDKIRHLEYQQLSTEDV

SEQ ID NO: 3 - protein, 91 aa, synthetic construct
MNSSSGTAISNTHSYYETDNKQEFENDRKPDTSNAVSEGSANKYSQEDAVCMLMAIKNLGDAYRRKNATKPSPSVLDKIRHLEYQQLSTED

SEQ ID NO: 4 - DNA, 6249 bp, Suid betaherpesvirus 2
  source 1..6249: mol_type = genomic DNA
ggccaaaagatttaccacctgttaatcggacccgtgatcacacatcacagagacacgtttccactaccgttcaatatagacatggcatattcatgcgacaatgccaacatattgccacacgtaaaggagaacctcgtgaagtgcatagagagcaatatcggtcccggacagtggatggtgtcatcatacaatggtttttttgacttttcggattcaaatgacataaatgagatgcagagaaaaatgtggttgtatgtaaaagagctggtttttggcgtggccctatacaatgatatttttaatcagtctatacaagtgtacagagtcgatgaggtggatgagacgatggaaggtattattctgacatataactgtaactatccgttattgttaaatctaaaatctaagatatataagtctagcgatttgtatctgctgttctattgtatatttgactccgtgaaggaaacgagtggcgagaccgtgagatgcgtacgtcgcacgcataaacgcgtatgtttgcggtctcttgtcagagatgacagtgagcagtcggaatttattccggattgttttattgattaactatggatatctcgtgtcatctaattctacgacatctacaacttcggcatctgaatcatcggttaccaccggatctcccggtactgtcagttctgaaacagcgacaccggaagtttctacggcttttgtgaacattaccggaaatttttcacaggaatacactgaagcaagcgatgatgaaaaatatccttttcgtgtatgcaatatggccgttggaacagatctatatagatttgataattatatcacttgtaataagtataatacggagactcagtattctgaaggtattttgttattgtttaagaaaaatatagttccgcacactttttttgttaggacgtataccaaggaattgtcgtttcaaacgacgtatagagacgttcatgtcatctatcttgttgataggtcttcatataaagtgcccgttcctgtagatgaagctggatatataaatttgaatggtcaatgtttttccgctgccgagattagaaaccagggaataaattatcgggtatatcataaagatgataatacaaacaatacaatgagattatatcgtttgaaatttggatctactattaatactcgttacatatccacgcctgaatttcagttcacatatggaacccattggttatataaaagctcatcttctattaattgcatagtaactgatactctggctaaatctgattacccatatgagaattttatactcggtacaggtgaatcggttgagatatcaccattttttaatggaacttctaaagaggtatttaatgaacagatgtactatttttcaatgaagaacaattacaccatgttagaaaaattagacgaacctaatggaccaaagaaaactatccctacgattgcttttctacaaaagggtgacacattgttttcatgggaggttaaggaacagactaattctcattgcaaatatacagcatggacaaaaaaacaccatgcattgcgagcagacatgacaaatagttatcattttatgatgaaggacactacagcaacaatggtaacaacaaagaacactataaatcttacgggcaccgaatacgagtgtgtgaaaaatgatattgaaaaatatattacagatacgtttcagaacaaatataacaatacacacaacaaaacagaaaactatagtgtttatgaaacaactacaggtctaatattagtttggcagcctattgttaggaaatcaattaaagagttgaaagaatttatcaatgaaacccaacatactcgttataagcgagaaattgatggaagtgactcacttgtttacgctagtttgcagtatatgtacgatgctttgagagagtatataaatgctggtttcgcccagatagctgaggcgtggtgcgaagatcagaaaagaaccaatgaagttctttccgaattagctaaaataagtccatctaatgtcatgtctgtaatctacgataaatcattatccgcaaaattagttggagatgcaatttccgtatcttcgtgtgtcaatgtaaatcagtctacggtgaaggtgcataaagatatgagaatatacgccaatgggacggcgaatagggagacgtgtttttctcgcccggtcgttacgtttgaattttcgaataataattcagttcaacaatttacgggtcaattggggccgaggaatgagattctgttaggcacgtatagagtggaaaaatgtgaacgtaacagtgttaaagttttttttgcgggtaaggaggcgtattttttttacaattacatttacacgaaaacggttaatatatctgatatcaatgtcgtagatacttttatccaccttaatataaagccgttagagaatacagactttgaggtattgcgaatgtattctaaaaatgagttagcgcaggctaacatatttgatctggaaagtcttttgagagatattaattcatataggagtgcgctttataatattgaatctaggatagctccaaggaaaccagattacgtctcaggtgtggattcttttctgcatgctcttggtataggtgcacccggagggttgggtgccgctctgggtatggcgacgggggcggtcacagatttcctgactggcattttttctttctttaagaatccattcgggggacttttttccatgctgttttttgttttgcttgttttcttgatattttccgtgtattacagacagaagaatatatatacaaacccggtaggtgcgttgtttccgtatgcaaatagttcttcgggaactgtaatatctaatacgcattcctattatgagacaaacaataaacaagagtttgagaatgacaggaaacctgatacgtccaatgccgtctctgagggcagtgccaataagtactctcaagaagatgccgtctgtatgttaatggctataaagaacctgggggatgcgtacagacggaagaatgccacaaagcctagcccgagcgtattagataagatacgtcacttggaatatcagcagctatccaccgaggacgtgtgacctgacgggcatgacattctttaatccatatataaagaaaaaatataaccctgacgttcccgccgcctgtcaatacatccagattcttcctaaggggattatacatgatggcgagaagggattgcttaaaaacctgtgcgagaaagaaccgcggatgttttatagggagaaacagtacctgttggataaagacatgaaatggccgtccctaaaaagaaaatgcagtgattctctatcatcatcgggggtgctgaaattccacgtgtacgacgccattgaatccgtgctgtgtaccgaggacattgaatatcttccgtacaactacagacatcatgtaattcctacgggaactgtgatacggttgttcggtaaaactgtaaatggtgtaaaaatctgcgtcaatgtgtttggacagctatcatacatatactgtgaagtgcccagcgaaacatacctgcatgatgttatgaaaaaatacatgtatgagaataacaagaattttaccttttctaccgcggtggacaataaattttctatgtatggattcaataggtctccggtgactgtgtataagatcacttttgctgactactttgccgcgcgcgaggtcgggcgtatgctcgaagcgcaaaaaataaaagtgtatgagacaagtgtggacgttctgtccagattctacatcgataatgactatcccagtttcggctggtacactgtacaaaaatattcaatcaatgaatacaaattctcgaatcaaagtcttgagattagctgttccgttaaggatctctgcccactcgatgaggacatatggcccgaatatgactgcctgacttttgatatagaatgtatgagtgaatcggggggattcccaaatgcctcggtacagtcagacattgtcattcagatatcagccgtactgcacaagacgggcagtgataatatggacgttcatctctttacattggggacttgcgaccccatagacgatgcccacatctatgagtttccctgtgaatacgaactcatctatgcttttctaatatttgtgaaacaggtatcgccagaaatcataacagggtataacataataaattttgatttgaactacttgatcatcagagccacgaaattgtataggctgggtgtcggagaatttacaaaattgaaaaagggactattgtgtatcaagtcgactaatgattttaaacaaggatttaactgtataaaaacaaaggtatacgcatccgggatgatatgcatagatttttaccctgtgtgtgtaggaaaaataacgtccgataattataaactggacaccattgccaacctgtgtctgggaaaacacaaggaagatatgccctataagcgtatacctatcgaattcatatctggttctggcggacgttgcaaagtgggcagatattgtatacaggattcgatgatagttaaggagttgttttgtaaattcaattaccatatggaagcatctgctatttctaagttggcctacttgcccatcaggaaggtgataaatgatggacagcagagaagggtgttcacgtgtatccttcatgagactagaaagagaaatatgctgatacccgacaagatagcgccgcgggagaccggcgaggacgagagctacaggggcgccactgttctggacccacacgtgggttactatgcttctccagtgattgtcatggattttgccagtttgtaccccagcataatgatagccaataatctgtgttactctaccctgatcttaaatgacgaggacgtgacggggatcgacgagaaagatattctgacggtgcatgtaaacaagaataccgtgtacaggttcgttaggagcagcgtcagggagtctatactcggcacgctgctgtctagatggctcaggaagagaaaggaagtgaaggcgcgcatgaaacgctgtgaggaccctatgttggcactgatacttgacaagcagcagcttgccctcaaggtgacgtgcaatgcgttttacggcttcacgggagccgtgcacggtctgctgccgtgtctccctctagcggcgtccatcaccagcatagggcgggacatgcttaggcagacgagtgactttatcaacaatgtcctttcgtctagagaatacgtgtcagagaagttcagtctctcagacggtgattttcagggggatttttccctgaatgtcatttacggagacacggatagcgtgttcgtgtgtgtcaagaacatcgtgcccgagaggctggccggcatatgtgctgacatagctgctcatgtgagctcgcagttgttcatcgagcctatcaaattagagtttgagaagatgttcctccccttgatgttgatatgcaagaagaggtatgtgggaaagatgtttgattcggacgagcttgtcatgaagggcgtagaactcgttagaaagacatcctgtccttttgtgaagaacattgtcaaggagatagtcagattgttgttctgggattccgaggttgccagggcggcggtcgagctctctcagatgagctgcgacgaagtgatacggaacggattgcctgcggggatacacaagatcattgacatcataattggcgcgagggataggttgtacaccaataaggtggatgtcaatgagcttgtcctgtccaccatgctgtctaaagagataagcatgtataagcagccaaacctgccgcacctccgtgtgatagagagattgagaaatagaaacgaggaggtgcccgtggtcggggacagagtgtcatatgtgctgatctcatccatggataagaatgttgcgaactatgagctctcggaggatcccctctacgtcatagagaacaatatacccataaacgccgaaagatactttgaacagctggtgaaagcggtctctaacataatctgcccaataatccccaaggacactctcaaaaaagaacggtttttattgggggtgataccggcgcgcgtatatctacacagtagttttagggacagtgtcattgttatgtaaaaaaaagaaccattaaataaattttacttgaaacagcacacattgctgagggtgtttctgtacttttcgtatttttcatttatgtcatccgggatgt

SEQ ID NO: 5 - DNA, 402 bp, Suid betaherpesvirus 2
  source 1..402: mol_type = genomic DNA
atgacagtgagcagtcggaatttattccggattgttttattgattaactatggatatctcgtgtcatctaattctacgacatctacaacttcggcatctgaatcatcggttaccaccggatctcccggtactgtcagttctgaaacagcgacaccggaagtttctacggcttttgtgaacattaccggaaatttttcacaggaatacactgaagcaagcgatgatgaaaaatatccttttcgtgtatgcaatatggccgttggaacagatctatatagatttgataattatatcacttgtaataagtataatacggagactcagtattctgaaggtattttgttattgtttaagaaaaatatagttccgcacactttttttgttaggacgtataccaaggaa

SEQ ID NO: 6 - DNA, 348 bp, Suid betaherpesvirus 2
  source 1..348: mol_type = genomic DNA
ttgatattttccgtgtattacagacagaagaatatatatacaaacccggtaggtgcgttgtttccgtatgcaaatagttcttcgggaactgtaatatctaatacgcattcctattatgagacaaacaataaacaagagtttgagaatgacaggaaacctgatacgtccaatgccgtctctgagggcagtgccaataagtactctcaagaagatgccgtctgtatgttaatggctataaagaacctgggggatgcgtacagacggaagaatgccacaaagcctagcccgagcgtattagataagatacgtcacttggaatatcagcagctatccaccgaggacgtgtga

SEQ ID NO: 7 - DNA, 2580 bp, Suid betaherpesvirus 2
  source 1..2580: mol_type = genomic DNA
atgacagtgagcagtcggaatttattccggattgttttattgattaactatggatatctcgtgtcatctaattctacgacatctacaacttcggcatctgaatcatcggttaccaccggatctcccggtactgtcagttctgaaacagcgacaccggaagtttctacggcttttgtgaacattaccggaaatttttcacaggaatacactgaagcaagcgatgatgaaaaatatccttttcgtgtatgcaatatggccgttggaacagatctatatagatttgataattatatcacttgtaataagtataatacggagactcagtattctgaaggtattttgttattgtttaagaaaaatatagttccgcacactttttttgttaggacgtataccaaggaattgtcgtttcaaacgacgtatagagacgttcatgtcatctatcttgttgataggtcttcatataaagtgcccgttcctgtagatgaagctggatatataaatttgaatggtcaatgtttttccgctgccgagattagaaaccagggaataaattatcgggtatatcataaagatgataatacaaacaatacaatgagattatatcgtttgaaatttggatctactattaatactcgttacatatccacgcctgaatttcagttcacatatggaacccattggttatataaaagctcatcttctattaattgcatagtaactgatactctggctaaatctgattacccatatgagaattttatactcggtacaggtgaatcggttgagatatcaccattttttaatggaacttctaaagaggtatttaatgaacagatgtactatttttcaatgaagaacaattacaccatgttagaaaaattagacgaacctaatggaccaaagaaaactatccctacgattgcttttctacaaaagggtgacacattgttttcatgggaggttaaggaacagactaattctcattgcaaatatacagcatggacaaaaaaacaccatgcattgcgagcagacatgacaaatagttatcattttatgatgaaggacactacagcaacaatggtaacaacaaagaacactataaatcttacgggcaccgaatacgagtgtgtgaaaaatgatattgaaaaatatattacagatacgtttcagaacaaatataacaatacacacaacaaaacagaaaactatagtgtttatgaaacaactacaggtctaatattagtttggcagcctattgttaggaaatcaattaaagagttgaaagaatttatcaatgaaacccaacatactcgttataagcgagaaattgatggaagtgactcacttgtttacgctagtttgcagtatatgtacgatgctttgagagagtatataaatgctggtttcgcccagatagctgaggcgtggtgcgaagatcagaaaagaaccaatgaagttctttccgaattagctaaaataagtccatctaatgtcatgtctgtaatctacgataaatcattatccgcaaaattagttggagatgcaatttccgtatcttcgtgtgtcaatgtaaatcagtctacggtgaaggtgcataaagatatgagaatatacgccaatgggacggcgaatagggagacgtgtttttctcgcccggtcgttacgtttgaattttcgaataataattcagttcaacaatttacgggtcaattggggccgaggaatgagattctgttaggcacgtatagagtggaaaaatgtgaacgtaacagtgttaaagttttttttgcgggtaaggaggcgtattttttttacaattacatttacacgaaaacggttaatatatctgatatcaatgtcgtagatacttttatccaccttaatataaagccgttagagaatacagactttgaggtattgcgaatgtattctaaaaatgagttagcgcaggctaacatatttgatctggaaagtcttttgagagatattaattcatataggagtgcgctttataatattgaatctaggatagctccaaggaaaccagattacgtctcaggtgtggattcttttctgcatgctcttggtataggtgcacccggagggttgggtgccgctctgggtatggcgacgggggcggtcacagatttcctgactggcattttttctttctttaagaatccattcgggggacttttttccatgctgttttttgttttgcttgttttcttgatattttccgtgtattacagacagaagaatatatatacaaacccggtaggtgcgttgtttccgtatgcaaatagttcttcgggaactgtaatatctaatacgcattcctattatgagacaaacaataaacaagagtttgagaatgacaggaaacctgatacgtccaatgccgtctctgagggcagtgccaataagtactctcaagaagatgccgtctgtatgttaatggctataaagaacctgggggatgcgtacagacggaagaatgccacaaagcctagcccgagcgtattagataagatacgtcacttggaatatcagcagctatccaccgaggacgtgtga

SEQ ID NO: 8 - protein, 116 aa, synthetic construct
MLIFSVYYRQKNIYTNPVGALFPYANSSSGTVISNTHSYYETNNKQEFENDRKPDTSNAVSEGSANKYSQEDAVCMLMAIKNLGDAYRRKNATKPSPSVLDKIRHLEYQQLSTEDV

SEQ ID NO: 9 - protein, 859 aa, synthetic construct
MTVSSRNLFRIVLLINYGYLVSSNSTTSTTSASESSVTTGSPGTVSSETATPEVSTAFVNITGNFSQEYTEASDDEKYPFRVCNMAVGTDLYRFDNYITCNKYNTETQYSEGILLLFKKNIVPHTFFVRTYTKELSFQTTYRDVHVIYLVDRSSYKVPVPVDEAGYINLNGQCFSAAEIRNQGINYRVYHKDDNTNNTMRLYRLKFGSTINTRYISTPEFQFTYGTHWLYKSSSSINCIVTDTLAKSDYPYENFILGTGESVEISPFFNGTSKEVFNEQMYYFSMKNNYTMLEKLDEPNGPKKTIPTIAFLQKGDTLFSWEVKEQTNSHCKYTAWTKKHHALRADMTNSYHFMMKDTTATMVTTKNTINLTGTEYECVKNDIEKYITDTFQNKYNNTHNKTENYSVYETTTGLILVWQPIVRKSIKELKEFINETQHTRYKREIDGSDSLVYASLQYMYDALREYINAGFAQIAEAWCEDQKRTNEVLSELAKISPSNVMSVIYDKSLSAKLVGDAISVSSCVNVNQSTVKVHKDMRIYANGTANRETCFSRPVVTFEFSNNNSVQQFTGQLGPRNEILLGTYRVEKCERNSVKVFFAGKEAYFFYNYIYTKTVNISDINVVDTFIHLNIKPLENTDFEVLRMYSKNELAQANIFDLESLLRDINSYRSALYNIESRIAPRKPDYVSGVDSFLHALGIGAPGGLGAALGMATGAVTDFLTGIFSFFKNPFGGLFSMLFFVLLVFLIFSVYYRQKNIYTNPVGALFPYANSSSGTVISNTHSYYETNNKQEFENDRKPDTSNAVSEGSANKYSQEDAVCMLMAIKNLGDAYRRKNATKPSPSVLDKIRHLEYQQLSTEDV

SEQ ID NO: 10 - DNA, 463 bp, Suid betaherpesvirus 2
  source 1..463: mol_type = genomic DNA
aacagtgttaaagttttttttgcgggtaaggaggcgtattttttttacaattacatttacacgaaaacggttaatatatctgatatcaatgtcgtagatacttttatccaccttaatataaagccgttagagaatacagactttgaggtattgcgaatgtattctaaaaatgagttagcgcaggctaacatatttgatctggaaagtcttttgagagatattaattcatataggagtgcgctttataatattgaatctaggatagctccaaggaaaccagattacgtctcaggtgtggattcttttctgcatgctcttggtataggtgcacccggagggttgggtgccgctctgggtatggcgacgggggcggtcacagatttcctgactggcattttttctttctttaagaatccattcgggggacttttttccatgctgttttttgttttgcttgttttct

SEQ ID NO: 11 - DNA, 1998 bp, Suid betaherpesvirus 2
  source 1..1998: mol_type = genomic DNA
atgagccgcatgtatccaaaatggacgtcctacatcacacaaataaaaataaacaatgatgttttgatatcgccgctagagtgcaaatgcctcctactcagcctaaaaatagaagtaccaataggatcatgtgttttgatgtgccaagataatgaaacctccgtatatgttccgttctcgttcctggacgtatccaacgacggtaagattaaagtaaacctaataaataaaactaacaattacatcagactaaaaagaacgcccattgtgctcaatgtcttcgccatatcatgcaacgtccccgtcatcatcaccacaccggtgaaggaactaaaggaattagtcataacaaatcacatggtatcgagcacaaagaatgaaacactaaccacctacaaggctggatcaaagatatgcttctgcatcaaaaccaattggaaaacaaaactgaaaagctctacgtttacaagattcgtgtcaagcgcatggacatttctgtacagcttctacaaggcgcatccgttcaattgcgcattcatcgaacacatatctctaaacggcggctatataaaaaatatatgtatatatgacaacagcgtgttgttatttgatatatgtatcgacaaaaacataaacgatccggagccgctaccgtcggacgtattcataagtattctttttcagaaccatgacgcagatctagttacgaacgataactgtgatccgtacataactaaaggagctcatccggcagaactctccgtctatgccaacactgacatgttgatcaaacccaaacagcaaacatcgatcacttatgatgtaaagtatacgtcgtcttcactaaacggagtatttctacctctgttttttaacggtattgaaatctacccagtcgtgtggaatagcgagtctaatctgtctctgccgttacgcggaggggccaatacagttcacatacggagaggacagttattagggaagatttatttattttcaaatgacttattcaaaacaagtgatatcacatcacaccctagcctcgaagaatcctcatccataatccttagtgaccatgtccctgaatcgagcggtcttggagacaggtcaagaccactactgtccctcagaagtataccacagacaagcacatcatccagcgtctcagaaacactgtccgtctctacgaaacctaagagggcgcagaaaaaaaccagttcttcggatagcacatatttttccacgccgtcaaagaaacctcataaaaaaaaagcagaccctttcggcacaccatatgcaaaacctgtcagcgaaaaggggtcacccaagaaacttcgaatatcctctggcgatacacaccgaaaaacagagccaccccccaaatccacacctatatcgattcaaaaactcaagatatcagacaccagcgacgacgaatcagaaatagaagatataaacgcacaaatcttttttatcactccccccgacgaagaacaggctaaaggtcccctgtcaacgcggacatcatctccgcgtatagacgatactcccatgggcacccctgtcgattttggattcgccgaatccgacgcggaaagagaaaagacacccatacccagtcccattatatttggagatgtcatagtgataagtagcagcgacgacgataatgaaaaaccgcacaagactagacccaggctcgtcaaacccagaccactggggaaatattccaagccgctcagctcggacgaggatgtgaacatagggctggctcagcaagccgtctccgccataaacgtacacggcgaatacactctgaggtgcaaacagttcctaaacgcctcagaaccgatctgcatcagacttcacagtctaggtctcacgcttcccatacagaacatgtgccccatgaaacaatgcatgctcacacaaacaaatgtgtattttgatatatctggcccagcgccgaaatacatcacaacactagaaataaaataa

SEQ ID NO: 12 - protein, 665 aa, Suid betaherpesvirus 2
MSRMYPKWTSYITQIKINNDVLISPLECKCLLLSLKIEVPIGSCVLMCQDNETSVYVPFSFLDVSNDGKIKVNLINKTNNYIRLKRTPIVLNVFAISCNVPVIITTPVKELKELVITNHMVSSTKNETLTTYKAGSKICFCIKTNWKTKLKSSTFTRFVSSAWTFLYSFYKAHPFNCAFIEHISLNGGYIKNICIYDNSVLLFDICIDKNINDPEPLPSDVFISILFQNHDADLVTNDNCDPYITKGAHPAELSVYANTDMLIKPKQQTSITYDVKYTSSSLNGVFLPLFFNGIEIYPVVWNSESNLSLPLRGGANTVHIRRGQLLGKIYLFSNDLFKTSDITSHPSLEESSSIILSDHVPESSGLGDRSRPLLSLRSIPQTSTSSSVSETLSVSTKPKRAQKKTSSSDSTYFSTPSKKPHKKKADPFGTPYAKPVSEKGSPKKLRISSGDTHRKTEPPPKSTPISIQKLKISDTSDDESEIEDINAQIFFITPPDEEQAKGPLSTRTSSPRIDDTPMGTPVDFGFAESDAEREKTPIPSPIIFGDVIVISSSDDDNEKPHKTRPRLVKPRPLGKYSKPLSSDEDVNIGLAQQAVSAINVHGEYTLRCKQFLNASEPICIRLHSLGLTLPIQNMCPMKQCMLTQTNVYFDISGPAPKYITTLEIK

SEQ ID NO: 13 - DNA, 1188 bp, Suid betaherpesvirus 2
  source 1..1188: mol_type = genomic DNA
atggcaaagcacaccgaaacaaacctgctgtctttaattgtctcgcccgacaaagcatacagtcctagcgaaagatcaaaagtgttatccctggggcatagtattcacacacgaaccgatgccgtcatactagccggagcagacaccttcacacctttcggggtgacgctttccgcatcatacgggaggctaggagcaccgatatttatcgtttctaatccaggtgccagaactagagaacccatacgcctcaacctaatagccataccgatcgtagaagtgactgataaatttttggtgtataccacactcaccgaagacaaggaaaataatagaaaggctccgagaagagccaaagccagccgcgtcgacgtatctcacgtcgacaccgaatcgactcgcacgctcaccatttccgtctacggactgcattggagcatcataacaaccccgagacagcacgaactcggagcaaatctatattttcagagtatatgggattgcgcatattacccagatgcattcagacccatgtatagcacacaccctgggatctttgttagacaagtgagagtgacaggtgacgatcagtacatggtatccctgaggtaccacccgacagaatctcccccgagccctccaaaagacctgatggtaaagatagaactacacatcaactccctagactccgaagcggtcatcctatacaaacgggccccgtctatggtaccggtcccgggcagaaactgcgtagatgtgctgtgtccggaggacgtcagactgatacccggggaggtaaaacatgtaaggatatcgaatcaatacatcactaaccaccacaccgggctattcatccccgaggtgttcgaggacgtcacagtatatccaaaagtatggaatgagggggaatcgctaatcgcatcatttctatcgaacaaggactccgtaagcgtagtgggtgaaggatacaagatcggctgcgtctacttcctaaaaaacccctttgtaaatagacagcacggaaacgacgaaccgtcccagagcatcacattgaaattagacacggccaataagacagtttcgtttctgggtatcgaaataccgttagacgcactggaaaccctcgtcatggatcaaaccgcgacatccacggcagaagtaacattacattgcgaaggacgcgtataa

SEQ ID NO: 14 - protein, 395 aa, Suid betaherpesvirus 2
MAKHTETNLLSLIVSPDKAYSPSERSKVLSLGHSIHTRTDAVILAGADTFTPFGVTLSASYGRLGAPIFIVSNPGARTREPIRLNLIAIPIVEVTDKFLVYTTLTEDKENNRKAPRRAKASRVDVSHVDTESTRTLTISVYGLHWSIITTPRQHELGANLYFQSIWDCAYYPDAFRPMYSTHPGIFVRQVRVTGDDQYMVSLRYHPTESPPSPPKDLMVKIELHINSLDSEAVILYKRAPSMVPVPGRNCVDVLCPEDVRLIPGEVKHVRISNQYITNHHTGLFIPEVFEDVTVYPKVWNEGESLIASFLSNKDSVSVVGEGYKIGCVYFLKNPFVNRQHGNDEPSQSITLKLDTANKTVSFLGIEIPLDALETLVMDQTATSTAEVTLHCEGRV

SEQ ID NO: 15 - DNA, 330 bp, Suid betaherpesvirus 2
  source 1..330: mol_type = genomic DNA
atggctatattattaaagatagacctacagtatgagcgtcaccgtacctttttatgtcatataattatgacctggatcgtggttttgtgttttgctttatttaacgtcatcgtggcgagtgagggattatcttttccctctcataagatcgatcataatacggaattttataaacagtcgtgccagtcccacgtattcgaggttgagttcacctcgttcaccacgatatggctactcataaatagccttttcctgttatcatctgttggaatttttttgaaattctggtgttacaaatcctttgcggaagagacggttaagggatactga

SEQ ID NO: 16 - protein, 109 aa, Suid betaherpesvirus 2
MAILLKIDLQYERHRTFLCHIIMTWIVVLCFALFNVIVASEGLSFPSHKIDHNTEFYKQSCQSHVFEVEFTSFTTIWLLINSLFLLSSVGIFLKFWCYKSFAEETVKGY

SEQ ID NO: 17 - DNA, 2049 bp, Suid betaherpesvirus 2
  source 1..2049: mol_type = genomic DNA
atggaactcaaacaccccttttactttctgcagattctgaacctcgtaagagcgtggaatgatatcgaaatcgtcaacaggtctctgacgtgcaaatcagagtcgtctaaccacactctagtacacccggggctgatcacctttaatttctactccggcgaggacagcgtcaggacatacagaatgccaaaatgtctgttcaccacggagatcgccgaaaggatcttcgcatccgtcaacctatcagaaacgctcgaggactacaaatcacgcttcaagaaacaattcttacctcccgtacacgcaaattataaggtgataatcaatcaaaatacgcccacaaatactaaccccgtatcagaaaccaataaccataattacgataataactatataaccctcaaggatttccaatggatacccatagaacaggactgtctgttgtataacgtaagcactgttatttttccgatcgtcatacaatgtaggaatcttaccttcaaggtgaaagatatatcaataacattttcattcaccaatcatttttttgtgttttcaataactatcggtaactctaaacccatcgatatagtcttcggaaacatagaacggatcaatgttaagagtccatataggaaagtagatctattgtatagacagaccgtcaccgatgatatgctcgtactggcaacggtaaacgatacatccaacgactactcgtcgcattttgggaaaaatatatttgatgaaatactccagacaaattacgttaacgtggagaccgcgatcggcttcacctcgcgattcatcgcgaattactttaaaaatgacctgtgccaaaatcgcaaggaagacttttacaagttgatatttctttacggactcagttctttcgcatacaaccaggtgaacgattccaggaagatattactgcctaaggtacttcagcttcacactgatatggccacgctaacattatttttaaggatatgcatcggggaggagatagaattcgtacacaaactaaaaaaagataacgtcacgtacgaaataataaaagatggaaataggtacaaaaatatcccgtcagatcccacatcacttggattcgtttcatatatgagccagcagaacgtatccgacgataacctgtccgaaatcgaaaaaagagccctaacactcttagatatgtacagaagaaccacaaacataacacgcgaggacaggaaaaccataaccatgctgcagctcatcgccaggaaaagtcacaacatgactaccaggaggacccttttgaacgtgttgactaccatgtgtacccccacggaaatccagaaatgggcggaaaatatccaggcccaccccgacctatccggtctctattcaccgtgtgctttcagtggacgattcgatctgacacccggttacctggagagactcgtgcggagtaccggtagagacacgtccatagctaaacgcatgcacaacctcattctaaaaaacaaaccaaaaacattggaggcctttaatctgagctgcgtgtccaccgaacgcgacctcatagtgataacccttgtaaatgtctcttatataatatcggatcgcataatggtgaaggggttgtcgtacataattacagcaactataataaatacagatatcatcataacggcggtgttctcgaacgagacctgtacggcgattcaatataaacacacagaaggaaccatcctgacgatggccaacaataccaacggcgaacatttattaaacaccgttctccttcagtacgattctgtggaaggggtcgtacggctcgtgtacatagatacaaaagaagagctgttacgtatcctcgatcccaataacgatataatcgtacgaagcccgaggactcattatcttttattgctgaacaacgggacgatattcaacgtatcgcccgtacacgtcttcttcagcgaaacatccatcatcctgcttttcgtatatttcatcatcgccttgttacttatattcgttttgtatatcatgtgccgcgtattttaa

SEQ ID NO: 18 - protein, 682 aa, Suid betaherpesvirus 2
MELKHPFYFLQILNLVRAWNDIEIVNRSLTCKSESSNHTLVHPGLITFNFYSGEDSVRTYRMPKCLFTTEIAERIFASVNLSETLEDYKSRFKKQFLPPVHANYKVIINQNTPTNTNPVSETNNHNYDNNYITLKDFQWIPIEQDCLLYNVSTVIFPIVIQCRNLTFKVKDISITFSFTNHFFVFSITIGNSKPIDIVFGNIERINVKSPYRKVDLLYRQTVTDDMLVLATVNDTSNDYSSHFGKNIFDEILQTNYVNVETAIGFTSRFIANYFKNDLCQNRKEDFYKLIFLYGLSSFAYNQVNDSRKILLPKVLQLHTDMATLTLFLRICIGEEIEFVHKLKKDNVTYEIIKDGNRYKNIPSDPTSLGFVSYMSQQNVSDDNLSEIEKRALTLLDMYRRTTNITREDRKTITMLQLIARKSHNMTTRRTLLNVLTTMCTPTEIQKWAENIQAHPDLSGLYSPCAFSGRFDLTPGYLERLVRSTGRDTSIAKRMHNLILKNKPKTLEAFNLSCVSTERDLIVITLVNVSYIISDRIMVKGLSYIITATIINTDIIITAVFSNETCTAIQYKHTEGTILTMANNTNGEHLLNTVLLQYDSVEGVVRLVYIDTKEELLRILDPNNDIIVRSPRTHYLLLLNNGTIFNVSPVHVFFSETSIILLFVYFIIALLLIFVLYIMCRVF

SEQ ID NO: 19 - DNA, 1032 bp, Suid betaherpesvirus 2
  source 1..1032: mol_type = genomic DNA
atgtccctaagcagggtggacagcattaatatcaaattatgggtgtctgcgatagtctgcgtctgtctgacttatctaaacgtcataatatacctgatatcctcgcacgtgcccggactcggcttcccctgcgcatattttgaaatcgttgacctctcctctataaatatgacgacatacaacgatatgcatacgctgacaccgcagttgtttatgacccctcccatgacagtgacctacgtagttttcacaatgatagtctacaccattatcacagcgtactacgtggcgtgctgcgtatccgtgtgtctgtactcgtccaaaaacacaagcggactgaatcactccaccaaacacataaaatgcctcggggacattttctcctgtctgcttttcatcctgtgtatggacacgtttcagcttttcatcaccacgctatcgttccggttgcccgcgatagccgcttttactttttgcatacacttcctgtgctctacgttctacacgggcactctgctcacacagtacgccagctacgagaaactaggcgccctctttaagcagaacaaatttctgtcgatgatggtcacggcgaagagtgtggtcgtgagcaccctcggtgtaaacctgggtatatgttgcatgatattctccctcacgctcttcctgggtgtggggaacagtttctacattaaaacggccaacataacattcgcatcaataaacctgttcatgattttaatgatattttattgcatactcatagaatcggtgttacacagagcaatgaagatgaattttggctttcactgtggtctatttataggactatgtggattattatatcccacgcacaaatatgaaagtatctatgcatcggagtggagctacaccatagatgtcaatctaggtatcgttggaatcgtttggattatcttcacgatctgtagaatcataagaacgctcttgccactcagacacagaaagcattataagccactcctagacgaagagactgaacctttaaaataa

SEQ ID NO: 20 - protein, 343 aa, Suid betaherpesvirus 2
MSLSRVDSINIKLWVSAIVCVCLTYLNVIIYLISSHVPGLGFPCAYFEIVDLSSINMTTYNDMHTLTPQLFMTPPMTVTYVVFTMIVYTIITAYYVACCVSVCLYSSKNTSGLNHSTKHIKCLGDIFSCLLFILCMDTFQLFITTLSFRLPAIAAFTFCIHFLCSTFYTGTLLTQYASYEKLGALFKQNKFLSMMVTAKSVVVSTLGVNLGICCMIFSLTLFLGVGNSFYIKTANITFASINLFMILMIFYCILIESVLHRAMKMNFGFHCGLFIGLCGLLYPTHKYESIYASEWSYTIDVNLGIVGIVWIIFTICRIIRTLLPLRHRKHYKPLLDEETEPLK

SEQ ID NO: 21 - DNA, 756 bp, Suid betaherpesvirus 2
  source 1..756: mol_type = genomic DNA
atggcgttgaaagactggctacgatcgaacataaaaacggaacacgatgaattgacaaacgaacagatcagcacacttttcgggatatctaggagctggattgattttattgatctatcatctatagatgtccactcattgagaaaaacgtactctattgtacgtgaactacggaataataatattgtgtttcctgaacacgactccattcacgtatggagtcacctgtgtacacctgacagagtgaaggttgttattctcggacaagatccctatccagacgagagaggttgcggtctcgcatttagcacaaggagagaccaacccgttccagaatctctgcgtaatatatacaaagaactggccaggagttcattttttcgaataccgcagagcggttgcttggagaaatggtgtgatgaaggggtcttgctgctaaatacagttttcaccgtaaccgcggggaagccatcctcccactccaacctcggctggcaggtattgtccgagaaggtcatcacatccatatcatgtaacctgaaaaacgtggtgttcctactgtggggaggacatgcgaaaaaatttatttccctgatagacgattcaaagcacctgatcctaacgtccagtcatccctcgcctaaggtgacgtcttcaaaagacccttttctcggaaacaatcactttatcaggaccaaccagtacttgcagagtcatgggaagacgcccgttaattggaacattttaacagactaa

SEQ ID NO: 22 - protein, 243 aa, Suid betaherpesvirus 2
MTEQCVQITRNCSGELDLNELDINEGTENHSLVDPKKISTIIKYEPPKYLFNIKLLKNSSFLNDSLLLRNNVQQVRTLLTLMKTDADTWSQVFRGYELCHGRDVLHTCVGENNTCATYNLRTLSYNPSPFTENVVGFELQKTSMNLNLTILIMLRNDFNNQSKVVRLPITSVAFFDALFNIIEFYRLNTESNINILTELAHYGYTFPELQVHRPILIVREHYKKHEIRNGTHYNTTYLNVTTQ

SEQ ID NO: 23 - skipped

SEQ ID NO: 24 - protein, 159 aa, Suid betaherpesvirus 2
MFKPARARAGGYNHAEIFLQSFQSNGSDLKYLYPFSLSQFLARPTAVPQLPHTPCNGTEQSGTSTGMFYYGEDKICVDIRNTDLSMYRIQAAVVTIPPMLASAVDMHYTLLHNHTSDNSTAQRFVSGLPIVGLQSLASVLDEEMHVILEKNYTVQGNSW

SEQ ID NO: 25 - protein, 96 aa, synthetic construct
MFPYANSSSGTVISNTHSYYETNNKQEFENDRKPDTSNAVSEGSANKYSQEDAVCMLMAIKNLGDAYRRKNATKPSPSVLDKIRHLEYQQLSTEDV

SEQ ID NO: 26 - protein, 96 aa, synthetic construct
MFPYANSSSGTAISNTHSYYETDNKQEFENDRKPDTSNAVSEGSANKYSQEDAVCMLMAIKNLGDAYRRKNATKPSPSVLDKIRHLEYQQLSTEDV

SEQ ID NO: 27 - protein, 154 aa, synthetic construct
MTVSSRNLFRIVLLINYGYLVSSNSTTSTTSASESSVTTGSPGTVSSETATPEVSTAFVNITGNFSQEYTEASDDEKYPFRVCNMAVGTDLYRFDNYITCNKYNTETQYSEGILLLFKKNIVPHTFFVRTYTKELSFQTTYRDVHVIYLVDRSS

SEQ ID NO: 28 - protein, 154 aa, synthetic construct
MTVSSRNLFRIVLLINYGYLVSSNSTTSTTSASESSVTTGSPGTVSSETATPEVSTAFVNITGNFSQEYTEASDDEKYPFRVCNMAVGTDLYRFDNYITCNKYNTETQYSEGILLLFKKNIVPHTFFVRTYTKELSFQTTYRDVHVIYLVDRPS

SEQ ID NO: 29 - protein, 155 aa, synthetic construct
MTKTVNISDINVVDTFIHLNIKPLENTDFEVLRMYSKNELAQANIFDLESLLRDINSYRSALYNIESRIAPRKPDYVSGVDSFLHALGIGAPGGLGAALGMATGAVTDFLTGIFSFFKNPFGGLFSMLFFVLLVFLIFSVYYRQKNIYTNPVGAL

SEQ ID NO: 30 - protein, 155 aa, synthetic construct; identical to SEQ ID NO: 29

SEQ ID NO: 31 - protein, 457 aa, synthetic construct
MYKVPVPVDEAGYINLNGQCFSAAEIRNQGINYRVYHKDDNTNNTMRLYRLKFGSTINTRYISTPEFQFTYGTHWLYKSSSSINCIVTDTIGKSDYPYENFILGTGESVEISPFFNGTSKEVFNEQMYYFSMKSNYTMLEKLDEPNGPKKTIPTIAFLQKGDTLFSWEVKEQTNSHCKYTAWTKKHHALRADMTNSYHFMMKDMTATMVTTKNTINLTGTEYECVKNDIEKYITDTFQSKYNNTHNKTENYSVYETTTGLILVWQPIVRKSIKELKEFINETQHTRYKREIDGSDSLVYASLQYMYDALREYINAGFAQIAEAWCEDQKRTNEVLSELAKINPSNVMSVIYDKSLSAKLVGDAISVSSYVNVNQSTVKVHKDMRIYANGTANRETCFSRPVVTFEFSNNNSVQQFTGQLGPRNEILLGTYRVEKCERNSVKVFFAGKEAYFFYNYIY

SEQ ID NO: 32 - protein, 101 aa, synthetic construct
MNSSSGTAISNTHSYYETDNKQEFENDRKPDTSNAVSEGSANKYSQEDAVCMLMAIKNLGDAYRRKNATKPSPSVLDKIRHLEYQQLSTEDHHHHHHHHHH

SEQ ID NO: 33 - protein, 10 aa, synthetic construct
HHHHHHHHHH

SEQ ID NO: 34 - protein, 876 aa, synthetic construct
MLFYIHIYKCTMAGSLKLRGSVLALWYLYQVALYSLSIAETGVTSPPNTATWSTESPLTGHYGTHDSSHGERGNNENRDSEEQNKNIYGSPSTFPYRVCSASGVGDVFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPELYLEPGWFWGSYRRRTTVNCELMDMFARSNPPFDFFVTATGDTVEMSPFWSGEDDHENKMHEKPWFVSVINNYKVVDYQNRGTVPLGKTRIFLDREEYTLSWEKHLKNMSYCPLTLWKAFYNGIQTDDSGSYHFVANDITASFTTSKEDMKEFNTTYHCLNEEIKAEIEKKYAKVNSTHSKYGDLKYFKTDGGLYLVWQPLIQNRLLDAKNKLNNETYSRRSRRQAESTTDPMMEMTGNGAGGEYSSENSITVAQVQYAYDNLRIRINNILEDLSKAWCREQHRAALVWNELSKINPTSVMSMIYNRPVSAKRIGDVISVSNCIVVDQTSVSLHKSLRLLSASDEKCFSRPPVTFKFMNDSTIYKGQLGVNNEILLTTTYLETCQENTEYYFQAKTDMYIYKNYEHLKTVPLSSITTLDTFIALNFTLLENVDFKVIELYTRDEKRLSNVFDIETMFREYNYYAQRVSGLRKDLLDLSTNRNQFVDAFGSLMDDLGAVGQTVVNAVSGVATLFSSIVTGFINFIKNPFGGMLMIIVVIGVLFAIYFLTKKTKIYETAPIKMIYPEIDKLKEREGKSEIAPISEEELERIVLAMHIHQQNSHMETKTRKDPKDSILTRAQNMLRKRSGYSNLKNAESVEMLNTL

SEQ ID NO: 35 - protein, 266 aa, synthetic construct
MLFYIHIYKCTMAGSLKLRGSVLALWYLYQVALYSLSIAETGVTSPPNTATWSTESPLTGHYGTHDSSHGERGNNENRDSEEQNKNIYGSPSTFPYRVCSASGVGDVFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPELYLEPGWFWGSYRRRTTVNCELMDMFARSNPPF

SEQ ID NO: 36 - protein, 276 aa, synthetic construct
MLFYIHIYKCTMAGSLKLRGSVLALWYLYQVALYSLSIAETGVTSPPNTATWSTESPLTGHYGTHDSSHGERGNNENRDSEEQNKNIYGSPSTFPYRVCSASGVGDVFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPELYLEPGWFWGSYRRRTTVNCELMDMFARSNPPFHHHHHHHHHH

SEQ ID NO: 37 - protein, 876 aa, synthetic construct
MLFYIHIYKCTMAGSLKLRRLALALWCQFQVALYALSKAEKSATSPANTQAWSTEALPIGQYGTYDSSHGERATSENRDEEEHNKNIYGSPSTFPYRVCSASGVGDIFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPDLYLEPGWFWGSYRRRTTVNCELMDMFARSNPPFDFFVTATGDTVEMSPFWSGEDDHENKMNEKLWFLSVINDYKVVDYQNRGTVPLGKTRIFLDREEYTLSWEKHLKNISYCPLTLWKAFYNGIQTEHSGSYHFVANDITASFTTSKEDMKDFNTTYHCLNDEIKAEIEKKYAKVNSTHSRYGDLQYFKTDGGLFLVWQPLIQNRLLDAKNKLNNETVSKRSRRQVDSTTGPMMEATGNGAGGEYSSENSITVAQVQYAYDNLRTRINNILEELSKAWCREQHRAALMWNELSKINPTSVMSMIYNRPVSAKRIGDVISVSNCIVVDQTSVSLHKSLRLLNAADDKCFSRPPVTFKFMNDSTIYKGQLGVNNEILLTTTYLETCQENTEYYFQAKTDMYIYKNYEHLKTVPLSSITTLDTFIALNFTLLENVDFKVIELYTRDEKRLSNVFDIETMFREYNYYAQRVSGLRKDLLDLSTNRNQFVDAFGSLMDDLGAVGQTVVNAVSGVATLFSSIVTGFINFIKNPFGGMLMIIVVIGVIFAIYFLTKKTKIYETAPIKMIYPEIDKLKEREGKSEIQPISEEELERIILAMHIHQQNSHMETKARKDPKDTILTRAQNMLRKRRGYTNLKNADSVEMLNTL

SEQ ID NO: 38 - protein, 299 aa, synthetic construct
MLFYIHIYKCTMAGSLKLRRLALALWCQFQVALYALSKAEKSATSPANTQAWSTEALPIGQYGTYDSSHGERATSENRDEEEHNKNIYGSPSTFPYRVCSASGVGDIFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPDLYLEPGWFWGSYRRRTTVNCELMDMFARSNPPFDFFVTATGDTVEMSPFWSGEDDHENKMNEKLWF

SEQ ID NO: 39 - protein, 309 aa, synthetic construct
MLFYIHIYKCTMAGSLKLRRLALALWCQFQVALYALSKAEKSATSPANTQAWSTEALPIGQYGTYDSSHGERATSENRDEEEHNKNIYGSPSTFPYRVCSASGVGDIFRFQTDHVCPDASDMVHSEGILLIYKQNIIPFMFRVRKYRKVVTTSTVYNGIYSDSITNQHTFYKSIEPWETEKMDTIYQCFNSLRLNTGGNLLTYVDRDDINMTVFLQPVDGVTPDVKRYGSQPDLYLEPGWFWGSYRRRTTVNCELMDMFARSNPPFDFFVTATGDTVEMSPFWSGEDDHENKMNEKLWFHHHHHHHHHH

SEQ ID NO: 40 - protein, 876 aa, synthetic construct
MLYSTHIYKCTMANSAKYRGCGQVLWMLFQVMFYFAVYGQYITTGAYDGNVNTEESEVTNHEYNYTDANTTAPEKKDPKEENQFYGSPSTFPYRVCSASGVGDIFRFQTEHKCPDTNDMVHNEGILLIYKQNIVPFMFRVRKYRKVVTTSTVYNGIYSDSVTNQHTFYKSIATWETEKMDTVYQCFNSLKLNVGNNLLTYVDRDDINMTVFLQPVDGVTTDVKRYGSQPDLYLEPGWFWGSYRRRTTVNCELTDMFARSNPPFDFFVTATGDTVEMSPFWTGPDDKENKLNEKPWFLTVIDGYKVVDYENRGTLPQGKTRIFLDREDYTLSWEKHLKNVSYCPLTLWKSFHNGIQTVHSSSYHFVANDITASFTVDKEELKDFNKTYSCLKDEIQNEMQKKFEKINSTHSKVGEMQYFKTEGGLFIVWQPLIQNRLIEARSRLNNEALRRVRRQTDGTSVTTTPEATVNNGNRTGANEESSENAISAAQVQYAYDNLRMRINNILEDLSKAWCREQHRAALMWNELSKINPTSVMTMIYNKPVSAKRIGDVISVSNCIIVDQNSVSLHKSLRVPNSEERCFSRPPVTFKFTNDSTVYKGQLGVNNEILLTTTYLETCQENTEYYFQAKKEMYVYKNYEHLKTVPLASITTLDTFIALNFTLLENVDFKVIELYTRDEKRLSNVFDIETMFREYNYYTQRVSGLRKDLLDLSTNRNQFVDAFGSLMDDLGAVGQTVVNAVSGVATLFSSIVTGFINFIKNPFGGMLIILVIIGVVVALYFLTKKTRHYEQAPVKMIYPDIEKLKEREKNENLKPISQEELDRIILAMHTYQQNSLEQLKEKKAPTPGFLSKAQNMLRKRSGYTNLVNSDSVEMLNSL

SEQ ID NO: 41 - protein, 317 aa, synthetic construct
MLYSTHIYKCTMANSAKYRGCGQVLWMLFQVMFYFAVYGQYITTGAYDGNVNTEESEVTNHEYNYTDANTTAPEKKDPKEENQFYGSPSTFPYRVCSASGVGDIFRFQTEHKCPDTNDMVHNEGILLIYKQNIVPFMFRVRKYRKVVTTSTVYNGIYSDSVTNQHTFYKSIATWETEKMDTVYQCFNSLKLNVGNNLLTYVDRDDINMTVFLQPVDGVTTDVKRYGSQPDLYLEPGWFWGSYRRRTTVNCELTDMFARSNPPFDFFVTATGDTVEMSPFWTGPDDKENKLNEKPWFLTVIDGYKVVDYENRGTLPQG

SEQ ID NO: 42 - protein, 327 aa, synthetic construct
MLYSTHIYKCTMANSAKYRGCGQVLWMLFQVMFYFAVYGQYITTGAYDGNVNTEESEVTNHEYNYTDANTTAPEKKDPKEENQFYGSPSTFPYRVCSASGVGDIFRFQTEHKCPDTNDMVHNEGILLIYKQNIVPFMFRVRKYRKVVTTSTVYNGIYSDSVTNQHTFYKSIATWETEKMDTVYQCFNSLKLNVGNNLLTYVDRDDINMTVFLQPVDGVTTDVKRYGSQPDLYLEPGWFWGSYRRRTTVNCELTDMFARSNPPFDFFVTATGDTVEMSPFWTGPDDKENKLNEKPWFLTVIDGYKVVDYENRGTLPQGHHHHHHHHHH

SEQ ID NO: 43 - protein, 6 aa, synthetic construct
HHHHHH

SEQ ID NO: 44 - protein, 4 aa, synthetic construct
HHHH

SEQ ID NO: 45 - protein, 5 aa, synthetic construct
HHHHH

SEQ ID NO: 46 - protein, 7 aa, synthetic construct
HHHHHHH

SEQ ID NO: 47 - protein, 8 aa, synthetic construct
HHHHHHHH

SEQ ID NO: 48 - protein, 9 aa, synthetic construct
HHHHHHHHH